(19) United States
(12) Patent Application Publication
(10) Pub. No.: US 2024/0398807 A1
(43) Pub. Date: Dec. 5, 2024

(54) COMPOSITIONS AND METHODS OF TREATING KIDNEY DISEASE AND FIBROSIS

(71) Applicant: Mitokinin, Inc., San Francisco, CA (US)

(72) Inventors: Nicholas Thomas Hertz, San Francisco, CA (US); Rishi Rakhit, Berkeley, CA (US)

(21) Appl. No.: 18/029,872

(22) PCT Filed: Oct. 4, 2021
(86) PCT No.: PCT/US2021/053358

§ 371 Date: Mar. 31, 2023

Related U.S. Application Data
(60) Provisional application No. 63/087,168, filed on Oct. 2, 2020.

(51) Int. Cl. A61K 31/519; A61P 9/10; A61P 13/12

(52) U.S. Cl. CPC A61K 31/519; A61P 9/10; A61P 13/12

(57) ABSTRACT

The present disclosure is directed to N-containing heteroaryl analogs, methods of making N-containing heteroaryl analogs, and methods of treating kidney diseases, fibrotic disorders, and reperfusion injuries using these analogs. This abstract is intended as a scanning tool for purposes of searching in the particular art and is not intended to be limiting of the present disclosure.

### CROSS-REFERENCE TO RELATED APPLICATIONS

This application claims the benefit of U.S. Application No. 63/087,168, filed on Oct. 2, 2020, the contents of which are hereby incorporated by reference in their entirety.

### FIELD

The disclosure relates to method of treating and/or preventing kidney disease, cisplatin induced ototoxicity and fibrosis comprising administering to a subject a pharmaceutically effective amount of a compound of the formulae disclosed herein BACKGROUND

Maintenance of mitochondrial function is essential for the health and survival of numerous cell types, including cardiomyoctes, hepatocytes, renal cells, and neurons. Aberrant mitochondrial quality control has been demonstrated to be an important factor in the development of kidney disease and fibrosis (Schapira, A. H. Mitochondrial disease. Lancet 379, 1825-1834, (2012); Chen, Y. and Dom, G. PINK1-Phosphorylated Mitofusin-2 Is a Parkin Receptor for Culling Damaged Mitochondria. Science 340, 471-475, (2013); Li, et al. Mitochondrial dysfunction in fibrotic disease. Cell Death Discovery 6, 80 (2020)), and also in ischemia/reperfusion injury (e.g., Wang and Zhou. Mitochondiral quality control mechanisms as molecular targets in cardiac ischemia-reperfusion injury. Acta Pharmaceutica Sinica B. In press. (2020)). The mitochondrial kinase PTEN Induced Kinase 1 (PINK1) plays an important role in the mitochondrial quality control processes by responding to damage at the level of individual mitochondria. The PINK1 pathway has also been linked to the induction of mitochondrial biogenesis and, critically, to the reduction of mitochondrially-induced apoptosis. See, e.g., Narendra, D. P. et al. PINK1 is selectively stabilized on impaired mitochondria to activate Parkin. PLoS Biol 8, e1000298 (2010), Wang, X., (2011). et al. PINK1 and Parkin target Miro for phosphorylation and degradation to arrest mitochondrial motility. Cell 147, 893-906, (2011), and Shin, J. H. et al. PARIS (ZNF746) repression of PGC-1alpha contributes to neurodegeneration in Parkinson's disease. Cell 144, 689-702, (2011).

Kidney disease, also known as renal disease, is typically a progressive loss in renal function over a period of months or years, although much more rapid onset cases can also occur (e.g., in acute kidney injury). Kidney disease is a major U.S. public health concern, with recent estimates suggesting that more than 26 million adults in the U.S. have the disease, including chronic kidney disease (CKD). The primary causes of kidney disease are diabetes and high blood pressure, which are responsible for up to two-thirds of the cases. In recent years, the prevalence of KD has increased due to a rising incidence of diabetes mellitus, hypertension (high blood pressure), and obesity, and also due to an aging population. Because kidney disease is co-morbid with cardiovascular disease, heart failure is a closely related health problem. For example, in the case of CKD, patients have an increased risk of death from cardiovascular events because CKD is thought to accelerate the development of heart disease (McCullough et al., Chronic kidney diseases, prevalence of premature cardiovascular disease, and relationship to short-term mortality, Am. Heart J., 2008; 156:277-283).

Progressive scarring (fibrosis) is a pathological feature of many chronic inflammatory diseases, and is an important cause of morbidity and mortality worldwide. Fibrosis is characterized by the accumulation of excess extracellular matrix components (e.g., collagen, fibronectin) that forms fibrous connective tissue in and around an inflamed or damaged tissue. Fibrosis may cause overgrowth, hardening, and/or scarring that disrupts the architecture of the underlying organ or tissue. While controlled tissue remodeling and scarring is part of the normal wound healing process promoted by transdifferentiation of fibroblasts into myofibroblasts, excessive and persistent scarring due to severe or repetitive injury or dysregulated wound healing (e.g., persistence of myofibroblasts) can eventually result in permanent scarring, organ dysfunction and failure, and even death.

Fibrotic changes can occur in vascular disorders (e.g., peripheral vascular disease, cardiac disease, cerebral disease) and in all main tissue and organ systems (e.g., lung, liver, kidney, heart, skin). Fibrotic disorders include a wide range of clinical presentations, including multisystemic disorders, such as systemic sclerosis, multifocal fibrosclerosis, and organ-specific disorders, such as pulmonary, liver, and kidney fibrosis (Rosenbloom et al., Ann. Intern. Med. 152:159, 2010; Wynn, Nat. Rev. Immunol. 4:583, 2004). While the etiology and causative mechanisms of individual fibrotic disorders may vary (e.g., ischemic event, exposure to a chemical, radiation, or infectious agent) and are poorly understood, they all share the common feature of abnormal and excessive deposition of extracellular matrix in affected tissues (Wynn and Ramalingam, Nat. Med. 18:1028, 2012). Currently, there are no effective therapies on the market in the U.S. for treating or preventing fibrotic disorders.

Ischemia/reperfusion (I/R) injury refers to damage to a tissue caused when the blood supply returns to the tissue after a period of ischemia (restriction in blood supply). The absence of oxygen and nutrients from the blood creates a condition in which the restoration of circulation results in inflammation and oxidative damage, rather than restoration of normal function. Ischemia/reperfusion injury can be associated with traumatic injury, including hemorrhagic shock, as well as many other medical conditions such as stroke or large vessel occlusion, and is a major medical problem. More particularly, ischemia/reperfusion injury is important in heart attacks, stroke, kidney failure following vascular surgery, post-transplantation injury and chronic rejection, as well as in various types of traumatic injury, where hemorrhage will lead to organ hypoperfusion, and then subsequent reperfusion injury during fluid resuscitation. Ischemia/reperfusion injury, or an injury due to reperfusion and ischemic events, is also observed in a variety of autoimmune and inflammatory diseases. Independently of other factors, ischemia/reperfusion injury leads to increased mortality.

Despite the gravity and widespread impact of disorders associated with mitochondrial dysfunction, including kidney diseases, fibrotic disorders, and reperfusion injury, compounds capable of selectively targeting the mitochondrial kinase PINK1 pathway and, thus, treating disorders associated with this pathway have remained elusive. Accordingly, there remains a need for compounds and compositions capable of modulating PINK1 kinase activity and methods of making and using same.

### SUMMARY

In accordance with the purpose(s) of the disclosure, as embodied and broadly described herein, the disclosure, in some embodiments, relates to N-containing heteroaryl compounds useful in the treatment of kidney disease such as, for example, chronic kidney disease or acute kidney injury (AKI), fibrotic disorders such as, for example, pulmonary fibrosis, liver fibrosis, heart fibrosis, mediastinal fibrosis, retroperitoneal cavity fibrosis, bone marrow fibrosis, skin fibrosis, scleroderma, pancreatic fibrillation, prostatic hyperplasia caused by fibrillation, and renal fibrosis, and reperfusion injuries such as, for example, reperfusion injuries induced by a mitochondrial disease (e.g., myocardial ischemia or stroke caused by Mitochondrial Encephalopathy, Lactic Acidosis, and Stroke-like episodes (MELAS)) and reperfusion injuries that are not induced by a mitochondrial disease (e.g., transplantation reperfusion, hepatic ischemia reperfusion, renal ischemia reperfusion, cerebral ischemia reperfusion).

The disclosure also relates to methods of treating a disorder in a subject in need thereof comprising administering to the subject a pharmaceutically effective amount of a compound having a structure represented by a formula:

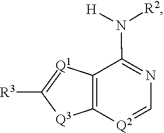

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; wherein R¹ is selected from C1-C6 haloalkyl, C1-C6 haloalkoxy, C1-C6 halohydroxyalkyl, and a structure represented by a formula:

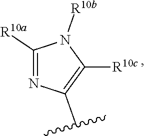

wherein each of R^{10a}, R^{10b}, and R^{10c}, when present, is independently selected from hydrogen and C1-C4 alkyl; wherein Q² is N or CH; wherein Q³ is CH₂ or NH; wherein R² is selected from C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy²; wherein n, when present, is 0, 1, or 2; wherein R¹², when present, is selected from hydrogen and C1-C4 alkyl; wherein Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and provided that when Q¹ is CR¹, R¹ is C1-C6 haloalkyl, and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl, or a pharmaceutically acceptable salt thereof, wherein the disorder is a kidney disease, a fibrotic disorder, cisplatin-induced toxicity or a reperfusion injury.

Also provided are kits comprising a compound having a structure represented by a formula:

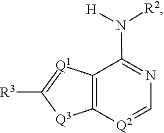

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; wherein R¹ is selected from C1-C6 haloalkyl, C1-C6 haloalkoxy, C1-C6 halohydroxyalkyl, and a structure represented by a formula:

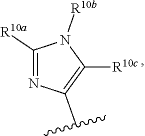

wherein each of R^{10a}, R^{10b}, and R^{10c}, when present, is independently selected from hydrogen and C1-C4 alkyl; wherein Q² is N or CH; wherein Q³ is CH₂ or NH; wherein R² is selected from C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy²; wherein n, when present, is 0, 1, or 2; wherein R¹², when present, is selected from hydrogen and C1-C4 alkyl; wherein Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and provided that when Q¹ is CR¹, R¹ is C1-C6 haloalkyl, and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl, or a pharmaceutically acceptable salt thereof, and one or more of: (a) an agent associated with the treatment of a kidney disease or a fibrotic disorder; (b) an agent associated with the treatment of a reperfusion injury; (c) instructions for administering the compound in connection with treating a kidney disease, a fibrotic disorder, and/or a reperfusion injury; and (d) instructions for treating a kidney disease, a fibrotic disorder, and/or a reperfusion injury.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE DRAWINGS

The accompanying figures, which are incorporated in and constitute a part of this specification, illustrate several embodiments and together with the description serve to explain the principles of the disclosure.

FIG. 1 shows representative data demonstrating that PINK1 activators 35985 and 40180 induce mitophagy in a dose dependent manner.

FIG. 2 shows representative data demonstrating that PINK1 activators 35985 and 40180 accelerate recruitment of Parkin to mitochondria.

FIG. 3A-C show representative data demonstrating that cisplatin induces PINK1 and its direct target pUb. Specifically, FIG. 3A and FIG. 3B shows that cistplatin causes mitochondrial damage in vivo as demonstrated by pS65-Ub increase (FIG. 3A) and induction of PINK1 (FIG. 3B). FIG. 3C shows the correlation between pUb and PINK1.

FIG. 4 shows representative data demonstrating that cisplatin induces reduction in mtDNA/nucDNA ratio.

FIG. 5A and FIG. 5B show representative data demonstrating that cistplatin-induced kidney damage is increased in PINK1 KO mice.

FIG. 6 shows representative data demonstrating that cisplatin does not induce a change in pS65 Ubiquitin in PINK1 KO mice.

FIG. 7 shows representative data demonstrating that cisplatin challenge increases mitochondrial-stress gene expression in PINK1 KO mice.

FIG. 8 shows representative data demonstrating a comparison of mouse plasma pharmacokinetics of 35985 and 40180.

FIG. 9 shows representative data demonstrating that 35985 ameliorates cisplatin-induced body-weight loss.

FIG. 10 shows representative data demonstrating that 35985 reduces kidney damage in cisplatin challenged mice as indicated by biomarker KIM-1.

FIG. 11A-C show representative data demonstrating that 35985 reduces expression of mitochondrial-stress related genes, including Tnfrsf12a (FIG. 11A), Gdf15 (FIG. 11B), and Atf3 (FIG. 11C).

FIG. 12 shows representative data demonstrating that 35985 ameliorates mtDNA loss upon cisplatin challenge.

FIG. 13 shows representative data demonstrating that 35985 reduces serum GDF15 levels as measured by ELISA.

FIG. 14 shows representative data demonstrating that 40180 reduces KIM-1 in cisplatin-challenged mice.

FIG. 15 show representative data demonstrating that 40180 reduces expression of mitochondrial-stress related genes.

FIG. 16 shows representative data demonstrating that 35985 restores kidney function in cisplatin-challenged mice.

FIG. 17 shows representative data demonstrating that 40270 reduces KIM-1 in cisplatin-challenged mice.

FIG. 18 shows representative data demonstrating that 41088 reduces weight loss in cisplatin-challenged mice.

FIG. 19 shows representative data demonstrating that 41088 reduces blood urea nitrogen (BUN) in cisplatin-challenged mice.

Additional advantages of the disclosure will be set forth in part in the description which follows, and in part will be obvious from the description, or can be learned by practice of the embodiments. The advantages of the embodiments will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

The present disclosure can be understood more readily by reference to the following detailed description of the disclosure and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting. Although any methods and materials similar or equivalent to those described herein can be used in the practice or testing of the present disclosure, example methods and materials are now described.

While embodiments of the present disclosure can be described and claimed in a particular statutory class, such as the system statutory class, this is for convenience only and one of skill in the art will understand that each embodiment of the present disclosure can be described and claimed in any statutory class. Unless otherwise expressly stated, it is in no way intended that any method or embodiment set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein may be different from the actual publication dates, which can require independent confirmation.

### A. Definitions

Listed below are definitions of various terms used to describe this disclosure. These definitions apply to the terms as they are used throughout this specification, unless otherwise limited in specific instances, either individually or as part of a larger group.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in other embodiments, to B without A (optionally including elements other than A); in yet other embodiments, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “about” means that the numerical value is approximate and small variations would not significantly affect the practice of the disclosed embodiments. Where a numerical limitation is used, unless indicated otherwise by the context, “about” means the numerical value can vary by ±10%, ±9%, ±8%, ±7%, ±6%, ±5%, ±4%, ±3%, ±2%, ±1% and remain within the scope of the disclosed embodiments.

The abbreviations used herein have their conventional meaning within the chemical and biological arts. The chemical structures and formulae set forth herein are constructed according to the standard rules of chemical valency known in the chemical arts.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a kidney disease, a fibrotic disorder, or a reperfusion injury, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various embodiments, a preparation can be administered therapeutically; that is, administered to treat an existing disease or condition. In further various embodiments, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In some embodiments, an IC₅₀ can refer to the concentration of a substance that is required for 50% inhibition in vivo, as further defined elsewhere herein.

As used herein, “EC₅₀,” is intended to refer to the concentration of a substance (e.g., a compound or a drug) that is results in a half-maximal response (i.e., 50% of the maximum response) of a biological process, or component of a process, including a protein, subunit, organelle, ribonucleoprotein, etc. In some embodiments, an EC₅₀ can refer to the concentration of a substance that is required to achieve 50% of the maximum response in vivo, as further defined elsewhere herein.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In broad embodiments, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain embodiments, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The terms “halo” and “halogen” as used herein refer to an atom selected from fluorine (fluoro, —F), chlorine (chloro, —Cl), bromine (bromo, —Br), and iodine (iodo, —I).

The term “alkyl,” as used herein, refers to a monovalent saturated, straight- or branched-chain hydrocarbon radical, having unless otherwise specified, 1-6 carbon atoms. Examples of alkyl radicals include, but are not limited to, methyl, ethyl, n-propyl, isopropyl, n-butyl, iso-butyl, sec-butyl, n-pentyl, tert-pentyl, neopentyl, sec-pentyl, 3-pentyl, sec-isopentyl, hexyl, 2-methylpentane, 3-methylpentane, 2,2-dimethylbutane, 2,3-dimentybutane and the like.

The term “haloalkyl” includes mono, poly, and perhaloalkyl groups where the halogens are independently selected from fluorine, chlorine, bromine, and iodine.

“Alkoxy” is an alkyl group which is attached to another moiety via an oxygen linker (—O(alkyl)). Non-limiting examples include methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

The term “9- to 10-membered carbocyclyl” means a 9- or 10-membered monocyclic, bicyclic (e.g., a bridged or spiro bicyclic ring), polycyclic (e.g., tricyclic), or fused hydrocarbon ring system that is saturated or partially unsaturated. The term “9- to 10-membered carbocyclyl” also includes saturated or partially unsaturated hydrocarbon rings that are fused to one or more aromatic or partically saturated hydrocarbon rings (e.g., dihydroindenyl and tetrahydronaphthalenyl). Bridged bicyclic cycloalkyl groups include, without limitation, bicyclo[4.3.1]decanyl and the like. Spiro bicyclic cycloalkyl groups include, e.g., spiro[3.6]decanyl, spiro[4.5]decanyl, spiro [4.4]nonyl and the like. Fused cycloalkyl rings include, e.g., decahydronaphthalenyl, dihydroindenyl, decahydroazulenyl, octahydroazulenyl, tetrahydronaphthalenyl, and the like. It will be understood that when specified, optional substituents on a carbocyclyl (e.g., in the case of an optionally substituted cycloalkyl) may be present on any substitutable position and, include, e.g., the position at which the carbocyclyl group is attached.

A cycloalkyl is a completely saturated carbocycle and includes e.g., cyclopropyl, cyclobutyl, cyclopentyl, cyclohexyl, cycloheptyl, and cyclooctyl.

The term “9-membered fused heterocyclyl” means a 9-membered saturated or partially unsaturated fused monocyclic heterocyclic ring comprising at least one oxygen heteroatom and optionally two to four additional heteroatoms independently selected from N, O, and S. The terms “heterocycle,” “heterocyclyl,” “heterocyclyl ring,” “heterocyclic group,” “heterocyclic moiety,” and “heterocyclic radical,” are used interchangeably herein. A heterocyclyl ring can be attached to its pendant group at any heteroatom or carbon atom that results in a stable structure. Examples of fused saturated or partially unsaturated heterocyclic radicals comprising at least one oxygen atom include, without limitation, dihydrobenzofuranyl, dihydrofuropyridinyl, octahydrobenzofuranyl, and the like. Where specified as being optionally substituted, substituents on a heterocyclyl (e.g., in the case of an optionally substituted heterocyclyl) may be present on any substitutable position and include, e.g., the position at which the heterocyclyl group is attached.

The term “5- or 6-membered heteroaryl” refers to a 5- or 6-membered aromatic radical containing 1-4 heteroatoms selected from N, O, and S. Nonlimiting examples include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. When specified, optional substituents on a heteroaryl group may be present on any substitutable position and, include, e.g., the position at which the heteroaryl is attached.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain embodiments, unless expressly indicated to the contrary, individual substituents can be further optionally substituted (i.e., further substituted or unsubstituted).

In some embodiments, a structure of a compound can be represented by a formula:

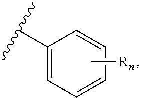

which is understood to be equivalent to a formula:

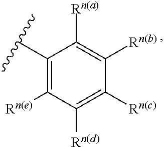

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further embodiments, a structure of a compound can be represented by a formula:

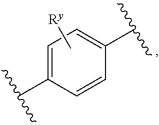

wherein R^{y} represents, for example, 0-2 independent substituents selected from A¹, A², and A³, which is understood to be equivalent to the groups of formulae:

wherein R^{y} represents 0 independent substituents

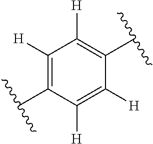

wherein R^{y} represents 1 independent substituent

| | | | |
|---|---|---|---|
| 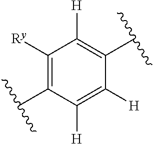 | 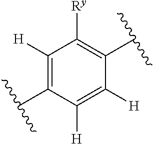 | 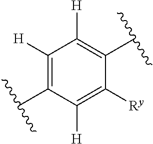 | 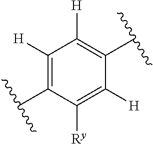 |

wherein R^{y} represents 2 independent substituents

| | | | |
|---|---|---|---|
| 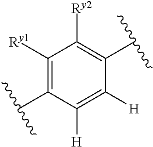 | 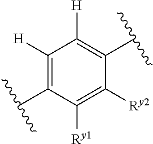 | 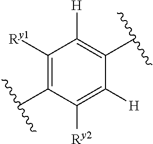 | 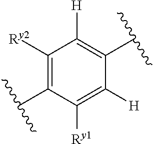 |
| 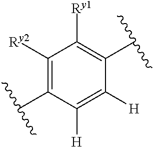 | 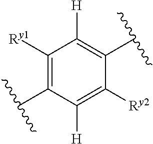 | 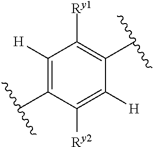 | 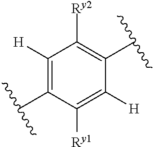 |
| 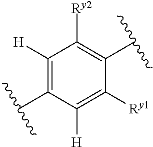 | 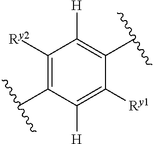 | 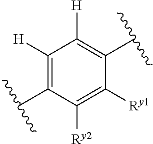 | 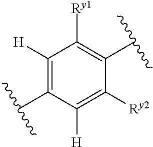 |

Again, by “independent substituents,” it is meant that each R substituent can be independently defined. For example, if in one instance R^{y1} is A¹, then R^{y2} is not necessarily A¹ in that instance.

In some further embodiments, a structure of a compound can be represented by a formula,

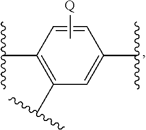

wherein, for example, Q comprises three substituents independently selected from hydrogen and A, which is understood to be equivalent to a formula:

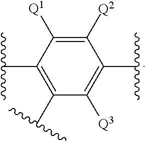

Again, by “independent substituents,” it is meant that each Q substituent is independently defined as hydrogen or A, which is understood to be equivalent to the groups of formulae:

wherein Q comprises three substituents independently selected from H and A

| | | | |
|---|---|---|---|
| 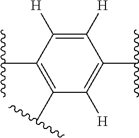 [SMILES: *c1ccc(*)c(*)c1] | 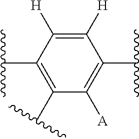 | 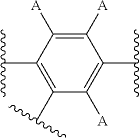 | 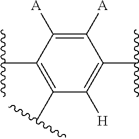 |
| 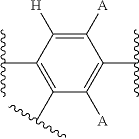 | 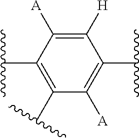 | 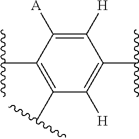 | 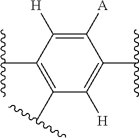 |

In some embodiments, the disclosed compounds exists as geometric isomers. “Geometric isomer” refers to isomers that differ in the orientation of substituent atoms in relationship to a cycloalkyl ring, i.e., cis or trans isomers. When a disclosed compound is named or depicted by structure without indicating a particular cis or trans geometric isomer form, it is to be understood that the name or structure encompasses one geometric isomer free of other geometric isomers, mixtures of geometric isomers, or mixtures enriched in one geometric isomer relative to its corresponding geometric isomer. When a particular geometric isomer is depicted, i.e., cis or trans, the depicted isomer is at least about 60%, 70%, 80%, 90%, 99% or 99.9% by weight pure relative to the other geometric isomer.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral and/or IV administration is preferred.

The terms “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The terms “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats or prevents a kidney disorder, a cisplatin-induced toxicity, ototoxicity, or reperfusion.

Disease, disorder, and condition are used interchangeably herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. Treatment may also be continued after symptoms have resolved, for example to delay their recurrence.

As used herein, the term “prevent” or “preventing” refers to precluding, averting, obviating, forestalling, stopping, or hindering something from initially happening, especially by advance action. It is understood that where reduce, inhibit, or prevent are used herein, unless specifically indicated otherwise, the use of the other two words is also expressly disclosed. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development. In some embodiments, methods of preventing a disease or disorder comprise a healthy human subject genetically predisposed to acquire the disease or disorder.

The term “effective amount” or “therapeutically effective amount” refers to an amount that is sufficient to achieve the desired result (e.g., that will elicit a biological or medical response of a subject e.g., a dosage of between 0.01-100 mg/kg body weight/day) or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

In further various embodiments, a preparation can be administered in a “prophylactically effective amount”; that is, an amount effective for prevention of a disease or condition.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. In some embodiments salt refers to inorganic and organic salts of compounds of the present invention. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitiate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977).

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). In some embodiments, the subject is a human, or a human in need of treatment.

The term “associated” or “associated with” in the context of a substance or substance activity or function associated with a disease (e.g., a protein associated disease, a symptom associated with a kidney disease, a fibrotic disorder, or with a reperfusion injury) means that the disease (e.g., the kidney disease, the fibrotic disorder, or the reperfusion injury) is caused by (in whole or in part), or a symptom of the disease is caused by (in whole or in part) the substance or substance activity or function. For example, a symptom of a disease or condition associated with a reduction in the level of PINK1 activity may be a symptom that results (entirely or partially) from a reduction in the level of PINK1 activity (e.g., loss of function mutation or gene deletion or modulation of PINK1 signal transduction pathway). As used herein, what is described as being associated with a disease, if a causative agent, could be a target for treatment of the disease. For example, a disease associated with PINK1, may be treated with an agent (e.g., compound as described herein) effective for decreasing the level of activity of PINK1.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g., PINK1). In embodiments contacting includes allowing a compound described herein to interact with a protein or enzyme that is involved in a signaling pathway.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In embodiments inhibition refers to reduction of a disease or symptoms of disease. In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The symbol “ [symbol] ” denotes the point of attachment of a chemical moiety to the remainder of a molecule or chemical formula.

The term “modulator” refers to a composition that increases or decreases the level of a target molecule or the function of a target molecule. In embodiments, the modulator is a modulator of PINK1. In embodiments, the modulator is a modulator of PINK1 and is a compound that reduces the severity of one or more symptoms of a disease associated with PINK1 (e.g., reduction of the level of PINK1 activity or protein associated with a kidney disease, a fibrotic disorder, or a reperfusion injury). In embodiments, a modulator is a compound that reduces the severity of one or more symptoms of a kidney disease, a fibrotic disorder, or a reperfusion injury that is not caused or characterized by PINK1 (e.g., by increase of PINK1 function) but may benefit from modulation of PINK1 activity (e.g., decrease in level of PINK1 or PINK1 activity).

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In embodiments, a patient is human.

“Disease” or “condition” refer to a state of being or health status of a patient or subject capable of being treated with a compound, pharmaceutical composition, or method provided herein. In embodiments, the disease is a disease related to (e.g., characterized by) an increase in the level of PINK1. In embodiments, the disease is a disease characterized by reperfusion injury. In embodiments, the disease is a disease characterized by neural cell death. In embodiments, the disease is a disease characterized by an increase in the level of PINK1 activity. In embodiments, the disease is a kidney disease. In embodiments, the disease is a fibrotic disorder. In embodiments, the disease is a reperfusion injury.

As used herein, the term “kidney disease” refers to a disease condition in which kidney function deteriorates, and can have a wide range of etiology, including via inflammatory and non-inflammatory diseases. As would be understood by one of ordinary skill in the art, a kidney disease can be induced by a variety of different things including, but not limited to, autoxic agents (e.g., cisplatin) and contrast dyes. Examples of kidney diseases that may be treated with a compound or method described herein include actue kidney disease (i.e., kidney disease in which there is a rapid loss of kidney function) and chronic kidney disease (i.e., a kidney disease in which there is a progressive loss in renal function over a period of time). Additional examples of kidney diseases include, but are not limited to, autosomal dominant polycystic kidney disease, diabetic nephropathy, hypertension-induced renal injury, crescentic glomerulonephritis, membranous nephropathy, membranous nephropathy, IgA nephropathy, amyloid A amyloidosis, and secondary nephrotic syndrome.

As used herein, the terms “fibrotic disorder” and “fibrotic disease” are used interchangeably, and refer to a disease or condition featuring progressive and/or irreversible fibrosis, wherein excessive deposition of extracellular matrix occurs in and around inflamed or damaged tissue. In certain embodiments, a fibrotic disorder or disease is associated with the persistent presence of myofibroblasts in and around fibrotic foci or lesions. Excessive and persistent fibrosis can progressively remodel and destroy normal tissue, which may lead to dysfunction and failure of affected organs, and ultimately death. A fibrotic disorder may affect any tissue in the body and is generally initiated by an injury and the transdifferentiation of fibroblasts into myofibroblasts. Examples of fibrotic disorders that may be treated with a compound or method described herein include pulmonary fibrosis (e.g., idiopathic pulmonary fibrosis), cystic fibrosis, liver fibrosis (e.g., nonalcoholic steatohepatitis (NASH) and cirrhosis), cardiac fibrosis, endomyocardial fibrosis, vascular fibrosis (e.g., atherosclerosis, stenosis, restenosis), atrial fibrosis, mediastinal fibrosis, myelofibrosis, retroperitoneal fibrosis, progressive massive fibrosis (e.g., lungs), chronic kidney disease, nephrogenic systemic fibrosis, Crohn's disease, hypertrophic scarring, keloid, scleroderma, systemic sclerosis (e.g., skin, lungs), athrofibrosis (e.g., knee, shoulder, other joints), Peyronie's disease, Dupuytren's contracture, adhesive capsulitis, organ transplant associated fibrosis, ischemia associated fibrosis, or the like.

As used herein, the terms “reperfusion injury,” “ischemia-reperfusion injury,” and “ischemia/reperfusion injury” are used interchangeably, and refer to a disease or condition in which there is a paradoxical exacerbation of cellular dysfunction and death, following restoration of blood flow to previously ischaemic tissues. Reestablishment of blood flow is essential to salvage ischaemic tissues; however, reperfusion itself paradoxically causes further damage, threatening function and viability of the organ. Reperfuion injury occurs in a wide range of organs including the heart, lung, kidney, gut, skeletal muscle, and brain, and may involve not only the ischaemic organ itself but may also induce systemic damage to distant organs, potentially leading to multi-system organ failure. Reperfusion injury is a multi-factorial process resulting in extensive tissue destruction. Examples of reperfusion injuries that may be treated with a compound or method described herein include, but are not limited to, renal ischemia (i.e., ischemia resulting from a deficiency of blood flow in one or both kidneys, or nephrons, usually due to functional constriction or actual obstruction of a blood vessel or surgical removal of the kidney; can be caused by, for example, hemorrhagic shock, septic shock, asphyxia also known as asphyxiation, cardiac arrest also known as cardiopulmonary arrest or circulatory arrest, respiratory arrest, respiratory failure, cardiogenic shock, aortic aneurysm, aortic aneurysm surgery, hypotension, dehydration, spinal shock, trauma, cadaveric renal transplantation, living related donor renal transplantation, liver transplantation, a liver disease, drug-induced renal ischemia, hydronephrosis, urethral obstruction, cardiopulmonary bypass surgery, radiocontrast administration, endovascular renal artery catheterization, renovascular stenosis, renal artery thrombosis, ureteral obstruction, hypoxia, and hypoxemia), retinal ischemia (i.e., ischemia in which the blood supply to the retinal cells is impaired, resulting in a deficiency of oxygenation to retinal tissues), and myocardial ischemia (i.e., ischemia in which there is a mismatch of oxygen supply and demand in the myocardium). In various aspects, the reperfusion injury can be induced by a mitochondrial disease (e.g., myocardial ischemia or stroke caused by Mitochondrial Encephalopathy, Lactic Acidosis, and Stroke-like episodes (MELAS)). In various further aspects, the reperfusion injury is not induced by a mitochondrial disease (e.g., transplantation reperfusion, hepatic ischemia reperfusion, renal ischemia reperfusion, cerebral ischemia reperfusion).

The terms “cisplatin-induced toxicity” as used herein refers to any disorder or toxicity of a subject caused by exposure of the subject to doses of cisplatin. In some embodiments, the cisplatin-induced toxicity is ototoxicity, gastrotoxicity, myelosuppression, and allergic reactions.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., kidney disease or fibrotic disorder therapies, including, for example, angiotensin converting enzyme inhibitors (e.g., benazepril, captopril, enalapril, fosinopril, lisinopril, moexipril, perindopril, quinapril, Ramipril, trandolapril), Angiotensin II Receptor Blockers (e.g., azilsartan, candesartan, eprosartan, irbesartan, losartan, olmesartan, telmisartan, and valsartan), nintedanib, pirfenidone, autotaxin inhibitors (e.g., carbamoyl imidazoles, benzonaphtyridines, pyridopyrimidines, PF-8380, pipemidic acids), or a peroxisome proliferator-activated receptor (PPAR) modulator (e.g., BADGE, EPI-001, INT-131, K-0533, S26948); or reperfusion injury therapies including, for example, hydrogen sulfide, cyclosporine, TR040303, superoxide dismutase, metformin, elamipretide, or a cannabinoid.

The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g., PINK1), and/or reducing, eliminating, or slowing the progression of disease symptoms (e.g., symptoms of a kidney disease, a fibrotic disorder, or a reperfusion injury). Determination of a therapeutically effective amount of a compound of the disclosure is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of cardiomyopathy or neurodegeneration such as Parkinson's disease and severity of such symptoms), kind of concurrent treatment, complications from the disease being treated or other health-related problems. Other therapeutic regimens or agents can be used in conjunction with the methods and compounds of Applicants' disclosure. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating a kidney disease or fibrotic disorder, including, for example, a pharmaceutically effective amount of angiotensin converting enzyme inhibitors (e.g., benazepril, captopril, enalapril, fosinopril, lisinopril, moexipril, perindopril, quinapril, Ramipril, trandolapril), Angiotensin II Receptor Blockers (e.g., azilsartan, candesartan, eprosartan, irbesartan, losartan, olmesartan, telmisartan, and valsartan), nintedanib, pirfenidone, autotaxin inhibitors (e.g., carbamoyl imidazoles, benzonaphtyridines, pyridopyrimidines, PF-8380, pipemidic acids), or a peroxisome proliferator-activated receptor (PPAR) modulator (e.g., BADGE, EPI-001, INT-131, K-0533, S26948, ASP1128); or in treating a reperfusion injury, including, for example, hydrogen sulfide, cyclosporine, TR040303, superoxide dismutase, metformin, elamipretide, or a cannabinoid, or with adjunctive agents that may not be effective alone, but may contribute to the efficacy of the active agent.

One of skill in the art will recognize that the appropriate dosage of the compositions and pharmaceutical compositions may vary depending on the individual being treated and the purpose. For example, the age, body weight, and medical history of the individual patient may affect the therapeutic efficacy of the therapy. Further, a lower dosage of the composition may be needed to produce a transient cessation of symptoms, while a larger dose may be needed to produce a complete cessation of symptoms associated with the disease, disorder, or indication. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response. Dosages may also depend on the strength of the particular composition, pharmaceutical composition, salt or analog chosen for the pharmaceutical composition.

The dose of the composition or pharmaceutical compositions may vary. The dose of the composition may be once per day. In some embodiments, multiple doses may be administered to the subject per day. In some embodiments, the total dosage is administered in at least two application periods. In some embodiments, the period can be an hour, a day, a month, a year, a week, or a two-week period. In an additional embodiment of the invention, the total dosage is administered in two or more separate application periods, or separate doses over the course of an hour, a day, a month, a year, a week, or a two-week period. In some embodiments, pharmaceutical compositions of the present disclosure can be administered once, twice, or three times in a 30 day period. In some embodiments, pharmaceutical compositions of the present disclosure can be administered once, twice, or three times in a 24 hour period.

Dosage may be measured in terms of mass amount of compound, salt, or analog per liter of liquid formulation prepared. One skilled in the art can increase or decrease the concentration of the polypeptide, salt, or analog in the dose depending upon the strength of biological activity desired to treat or prevent any above-mentioned disorders associated with the treatment of subjects in need thereof. For instance, some embodiments of the invention can include up to 0.001 grams of compound, salt, or analog per 5 mL of liquid formulation and up to about 1 grams of compound, salt, or analog per 5 mL of liquid formulation.

In embodiments, co-administration includes administering one active agent within about 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In embodiments, the active and/or adjunctive agents may be linked or conjugated to one another. In embodiments, the compounds described herein may be combined with treatments for neurodegeneration such as surgery. In embodiments, the compounds described herein may be combined with treatments for cardiomyopathy such as surgery.

“PINK1” is used according to its common, ordinary meaning and refers to proteins of the same or similar names and functional fragments and homologs thereof. The term includes and recombinant or naturally occurring form of PINK1 (e.g., “PTEN induced putative kinase 1”; Entrez Gene 65018, OMIM 608309, UniProtKB Q9BXM7, and/or RefSeq (protein) NP_115785.1, the contents of which are incorporated by reference in their entireties). The term includes PINK1 and variants thereof that maintain PINK1 activity (e.g., within at least about 30%, 40%, 50%, 60%, 70%, 80%, 90%, 95%, or 100% activity as compared to PINK1).

The term “neo-substrate” refers to a composition that is structurally similar to a composition that is a substrate for a protein or enzyme during the normal functioning of the protein or enzyme, but that is structurally distinct from the normal substrate of the protein or enzyme. In some embodiments, the composition comprises a neo-substrate. In embodiments, the neo-substrate is a better substrate for the protein or enzyme than the normal substrate (e.g., the reaction kinetics are better (e.g., faster), binding is stronger, turnover rate is higher, reaction is more productive, equilibrium favors product formation). In embodiments, the neo-substrate is a derivative of adenine, adenosine, AMP, ADP, or ATP. In embodiments, the neo-substrate is a substrate for PINK1. In embodiments, the neo-substrate is an N6 substituted adenine, adenosine, AMP, ADP, or ATP.

The term “derivative” as applied to a phosphate containing, monophosphate, diphosphate, or triphosphate group or moiety refers to a chemical modification of such group wherein the modification may include the addition, removal, or substitution of one or more atoms of the phosphate containing, monophosphate, diphosphate, or triphosphate group or moiety. In embodiments, such a derivative is a prodrug of the phosphate containing, monophosphate, diphosphate, or triphosphate group or moiety, which is converted to the phosphate containing, monophosphate, diphosphate, or triphosphate group or moiety from the derivative following administration to a subject, patient, cell, biological sample, or following contact with a subject, patient, cell, biological sample, or protein (e.g., enzyme). In some embodiments, a triphosphate derivative is a gamma-thio triphosphate. In some embodiments, a derivative is a phosphoramidate. In some embodiments, the derivative of a phosphate containing, monophosphate, diphosphate, or triphosphate group or moiety is as described in Murakami et al. J. Med Chem., 2011, 54, 5902; Sofia et al., J. Med Chem. 2010, 53, 7202; Lam et al. ACC, 2010, 54, 3187; Chang et al., ACS Med Chem Lett., 2011, 2, 130; Furman et al., Antiviral Res., 2011, 91, 120; Vernachio et al., ACC, 2011, 55, 1843; Zhou et al, AAC, 2011, 44, 76; Reddy et al., BMCL, 2010, 20, 7376; Lam et al., J. Virol., 2011, 85, 12334; Sofia et al., J. Med. Chem., 2012, 55, 2481, Hecker et al., J. Med. Chem., 2008, 51, 2328; or Rautio et al., Nature Rev. Drug. Discov., 2008, 7, 255, all of which are incorporated herein by reference in their entirety for all purposes.

The term “mitochondrial dysfunction” is used in accordance with its ordinary meaning and refers to aberrant activity of function of the mitochondria, including for example aberrant respiratory chain activity, reactive oxygen species levels, calcium homeostasis, programmed cell death mediated by the mitochondria, mitochondrial fusion, mitochondrial fission, mitophagy, lipid concentrations in the mitochondrial membrane, and/or mitochondrial permeability transition.

As used herein, the term “mitochondrial disease” refers to a disease, disorder, or condition in which the function of a subject's mitochondria becomes impaired or dysfunctional. Examples of mitochondrial diseases that may be treated with a compound or method described herein include Alzheimer's disease, amyotrophic lateral sclerosis, Asperger's Disorder, Autistic Disorder, bipolar disorder, cancer, cardiomyopathy, Charcot Marie Tooth disease (CMT, including various subtypes such as CMT type 2b and 2b), Childhood Disintegrative Disorder (CDD), diabetes, diabetic nephropathy, epilepsy, Friedreich's Ataxia (FA), Hereditary motor and sensory neuropathy (HMSN), Huntington's Disease, Keams-Sayre Syndrome (KSS), Leber's Hereditary Optic Neuropathy (LHON, also referred to as Leber's Disease, Leber's Optic Atrophy (LOA), or Leber's Optic Neuropathy (LON)), Leigh Disease or Leigh Syndrome, macular degeneration, Mitochondrial Myopathy, Lactacidosis, and Stroke (MELAS), mitochondrial neurogastrointestinal encephalomyophathy (MNGIE), motor neuron diseases, Myoclonic Epilepsy With Ragged Red Fibers (MERRF), Neuropathy, ataxia, retinitis pigmentosa, and ptosis (NARP), Parkinson's disease, Peroneal muscular atrophy (PMA), Pervasive Developmental Disorder Not Otherwise Specified (PDD-NOS), renal tubular acidosis, Rett's Disorder, Schizophrenia, and types of stroke associated with mitochondrial dysfunction.

The term “oxidative stress” is used in accordance with its ordinary meaning and refers to aberrant levels of reactive oxygen species.

As used herein, the term “animal” includes, but is not limited to, humans and non-human vertebrates such as wild, domestic, and farm animals.

As used herein, the term “antagonize” or “antagonizing” means reducing or completely eliminating an effect, such as an activity of GPR109a.

As used herein, the phrase “anti-receptor effective amount” of a compound can be measured by the anti-receptor effectiveness of the compound. In some embodiments, an anti-receptor effective amount inhibits an activity of the receptor by at least 10%, by at least 20%, by at least 30%, by at least 40%, by at least 50%, by at least 60%, by at least 70%, by at least 80%, by at least 90%, or by at least 95%. In some embodiments, an “anti-receptor effective amount” is also a “therapeutically effective amount” whereby the compound reduces or eliminates at least one effect of GPR109a. In some embodiments, the effect is the B-arrestin effect. In some embodiments, the effect is the G-protein mediated effect.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the terms “individual,” “subject” or “patient,” used interchangeably, means any animal, including mammals, such as mice, rats, other rodents, rabbits, dogs, cats, swine, cattle, sheep, horses, or primates, such as humans.

As used herein, the phrase “inhibiting activity,” such as enzymatic or receptor activity means reducing by any measurable amount the activity of PINK1.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “prodrug” means a derivative of a known direct acting drug, which derivative has enhanced delivery characteristics and therapeutic value as compared to the drug, and is transformed into the active drug by an enzymatic or chemical process. The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds of the disclosure as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the disclosure. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

As used herein, the term “purified” means that when isolated, the isolate contains at least 90%, at least 95%, at least 98%, or at least 99% of a compound described herein by weight of the isolate.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It should be noted that any embodiment of the disclosure can optionally exclude one or more embodiments for purposes of claiming the subject matter.

In some embodiments, the compounds, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

### B. Compounds

In various embodiments, the disclosure relates to compounds useful in treating disorders associated with PINK1 kinase activity such as, for example, a kidney disease, a fibrotic disorder, and/or a reperfusion injury.

In various embodiments, the compounds are useful in treating a disorder associated with PINK1 kinase activity in a mammal. In further embodiments, the compounds are useful in treating PINK1 kinase activity in a human.

It is contemplated that each disclosed derivative can be optionally further substituted. It is also contemplated that any one or more derivative can be optionally omitted from the disclosure. It is understood that a disclosed compound can be provided by the disclosed methods. It is also understood that the disclosed compounds can be employed in the disclosed methods of using.

### 1. Structure

In some embodiments, provided are compounds having a structure represented by a formula:

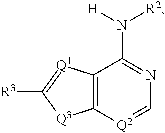

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; wherein R¹ is selected from C1-C6 haloalkyl, C1-C6 haloalkoxy, C1-C6 halohydroxyalkyl, and a structure represented by a formula:

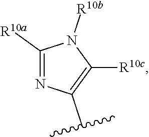

wherein each of R^{10a}, R^{10b}, and R^{10c}, when present, is independently selected from hydrogen and C1-C4 alkyl; wherein Q² is N or CH; wherein Q³ is CH₂ or NH; wherein R² is selected from C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy²; wherein n, when present, is 0, 1, or 2; wherein R¹², when present, is selected from hydrogen and C1-C4 alkyl; wherein Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and provided that when Q¹ is CR¹, R¹ is C1-C6 haloalkyl, and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b} when present, is hydrogen, and Cy¹ is a 6-membered aryl or furanyl, then Q¹ is CH and R³ is not a C1-C6 haloalkyl, or a pharmaceutically acceptable salt thereof.

In some embodiments, provided are compounds having a structure represented by a formula:

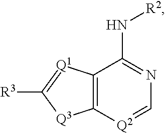

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

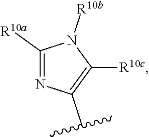

wherein each of R^{10a}, R^{10b} and R^{10c} when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b} when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl, or a pharmaceutically acceptable salt thereof.

In some embodiments, provided is a compound having a structure:

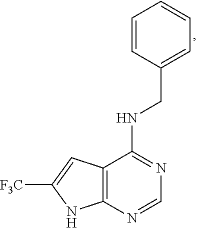 [SMILES: FC(F)(F)c1cc2c(NCc3ccccc3)ncnc2[nH]1]

or a pharmaceutically acceptable salt thereof.

In some embodiments, provided are compounds having a structure represented by a formula:

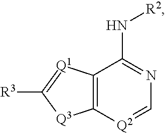

wherein Q¹ is N or CH and R³ is a 3- to 6-membered cycloalkyl or a C1-C6 haloalkyl, C1-C6 haloalkoxy, C1-C6 halohydroxyl, CF₃, CCl₃, CBr₃; or wherein Q¹ is CR¹ and R³ is hydrogen; R¹ is C1-C6 haloalkyl, C1-C6 haloalkoxy, C1-C6 halohydroxy, or a structure represented by a formula:

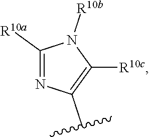

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C5 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, or a pharmaceutically acceptable salt thereof.

In some embodiments, provided are compounds selected from:

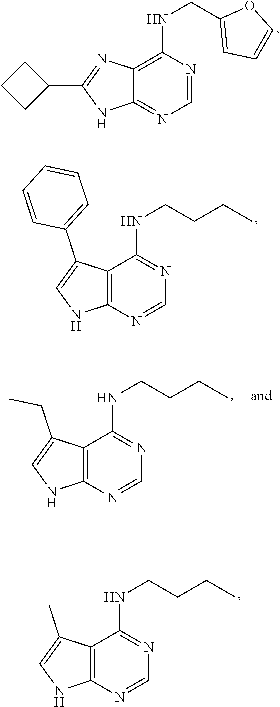

or a pharmaceutically acceptable salt thereof.

In some embodiments, provided are compounds selected from:

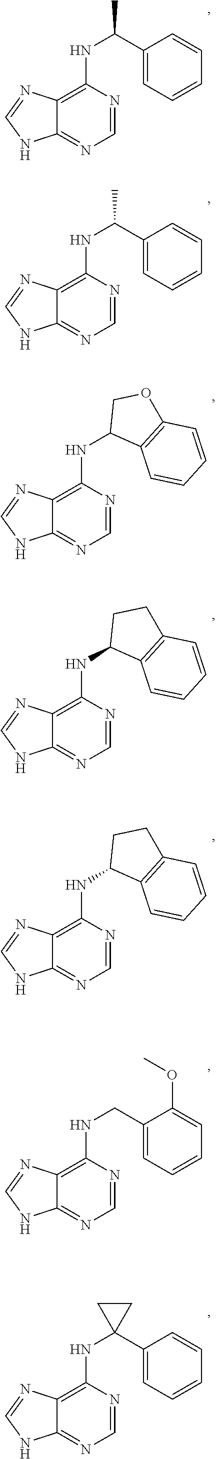

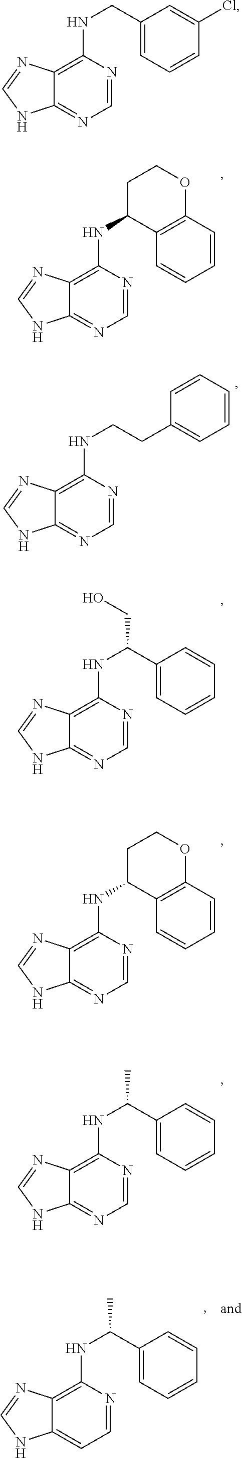

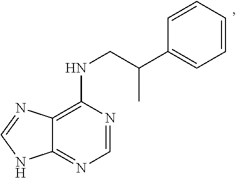 [SMILES: CC(CNc1ncnc2[nH]cnc12)c1ccccc1]

or a pharmaceutically acceptable salt thereof.

In some embodiments, provided are compounds having a structure represented by Formula I:

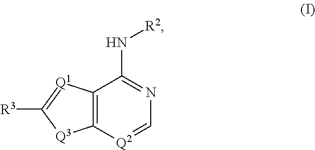

wherein Q¹ is N or CH and R³ is a 3- to 6-membered cycloalkyl or a C1-C6 haloalkyl, C1-C6 haloalkoxy, C1-C6 halohydroxyl, CF₃, CCl₃, CBr₃; or wherein Q¹ is CR¹ and R³ is hydrogen; wherein Q² is CH or N; wherein Q³ is CH₂ or NH; R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said C₁-C₆alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}; Ra, when present, is H, (C₁-C₄)alkyl, or (C₁-C₄)alkoxy; each occurrence of R^{b}, when present, is independently halo, halo(C₁-C₄)alkyl, (C₁-C₄)alkoxy, or halo(C₁-C₄)alkoxy; R² is (C₁-C₆)alkyl, a 9-membered oxygen-containing fused heterocycle, or a 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, 3- to 6-membered cycloalkyl, halo, halo(C₁-C₄)alkyl, halo (C₁-C₄)alkoxy or pharmaceutically acceptable salts thereof.

Thus, in various embodiments, the present disclosure provides a compound of Formula I:

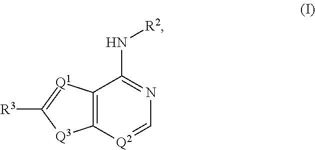

or a pharmaceutically acceptable salt thereof, wherein the variables are as defined above.

In further embodiments, R¹ in the compound of Formula I is (C₁-C₄)alkyl, halo(C₁-C₄)alkyl, 5- or 6-membered heteroaryl, or phenyl, wherein said halo(C₁-C₄)alkyl is optionally substituted with a OR^{a} group, and wherein said 5- or 6-membered heteroaryl is optionally substituted with a R^{b} group; Ra, when present, is H or (C₁-C₄)alkoxy; R^{b}, when present, is (C₁-C₄)alkyl; each occurrence of R^{d} and R^{e}, when present, is independently selected from halo and (C₁-C₄)alkoxy, and wherein the remaining variables are as described above for Formula I.

In further embodiments, R¹ in the compound of Formula I is (C₁-C₄)alkyl, halo(C₁-C₃)alkyl, 5-membered nitrogen containing heteroaryl, or phenyl, wherein said halo(C₁-C₃)alkyl is optionally substituted with a OR^{a} group, wherein said 5-membered nitrogen containing heteroaryl is optionally substituted with a (C₁-C₄)alkyl group, and wherein the remaining variables are as described above for Formula I or the second embodiments.

In further embodiments, R¹ is methyl, ethyl, —CF₃, —CH₂CF₃, 1,1,1-trifluoropropanol-3-yl, 2-ethoxy-1,1,1-trifluoropropane-3-yl, phenyl, or pyrazolyl, wherein said pyrazolyl is optionally substituted with a methyl group, and wherein the remaining variables are as described above for Formula I or the second or third embodiments.

In further embodiments, the compound of Formula I is of the Formula II:

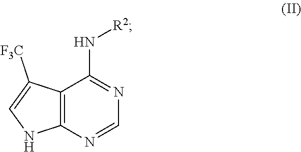

or a pharmaceutically acceptable salt thereof, wherein the variables are as described above for Formula I or the second embodiment.

In further embodiments, the compound of Formula I is of the Formula III:

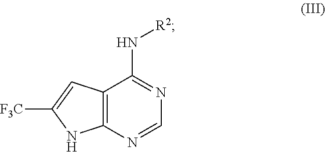

or a pharmaceutically acceptable salt thereof, wherein the variables are as described above for Formula I or the second embodiments.

In further embodiments, R² in the compound of Formula I, II, or III is (C₁-C₄)alkyl, benzofuranyl, dihydro-TH-indenyl, or tetrahydronaphthalenyl, wherein said (C₁-C₄)alkyl is optionally substituted with a R^{C} group, wherein said benzofuranyl, dihydro-TH-indenyl, and tetrahydronaphthalenyl are each optionally and independently substituted with 1 to 3 groups independently selected from R^{d}, and wherein the remaining variables are as described above for Formula I or the second, third, or fourth embodiments.

In further embodiments, each occurrence of R^{c}, when present, in the compound of Formula I, II, III is phenyl, cyclopropyl, pyridinyl, pyrazinyl, or pyrimidinyl, each of which are optionally and independently substituted with 1 to 2 groups independently selected from R^{e}, and wherein the remaining variables are as described above for Formula I or the second, third, fourth, or sixth embodiments.

In further embodiments, each occurrence of R^{e}, when present, in the compound of Formula I, II, or III is chloro, fluoro, or methoxy, and wherein the remaining variables are as described above for Formula I or the second, third, fourth, sixth, or seventh embodiments.

In further embodiments, each occurrence of R^{d}, when present, in the compound of Formula I, II, or III is (C₁-C₄)alkoxy, and wherein the remaining variables are as described above for Formula I or the second, third, fourth, sixth, seventh, or eighth embodiments. Alternatively, each occurrence of R^{d}, when present, in the compound of Formula I, II, or III is methoxy, and wherein the remaining variables are as described above for Formula I or the second, third, fourth, sixth, seventh, or eighth embodiments.

In further embodiments, R² in the compound of Formula I, II, or III is (C₁-C₄)alkyl optionally substituted with phenyl or pyrimidine-5-yl, wherein said phenyl is optionally substituted with 1 to 2 independently selected halo groups, and wherein the remaining variables are as described above for Formula I or the second, third, fourth, sixth, or seventh embodiments.

In further embodiments, provided are compounds having a structure represented by a formula:

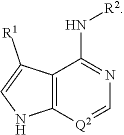

In further embodiments, provided are compounds having a structure represented by a formula:

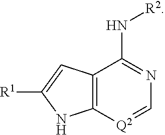

wherein R¹ is a 3- to 6-membered cycloalkyl or a C1-C6 haloalkyl, C1-C6 haloalkoxy, C1-C6 halohydroxyl. In some embodiments, Riis independently selected from: CCl₃, CF₃, or CBr₃.

In further embodiments, provided are compounds having a structure represented by a formula:

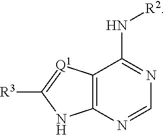

In further embodiments, provided are compounds having a structure represented by a formula:

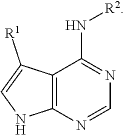

In further embodiments, provided are compounds having a structure represented by a formula:

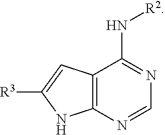

wherein R¹ is a 3- to 6-membered cycloalkyl or a C1-C6 haloalkyl, C1-C6 haloalkoxy, C1-C6 halohydroxyl. In some embodiments, Riis independently selected from: CCl₃, CF₃, or CBr₃.

In further embodiments, provided are compounds having a structure represented by a formula:

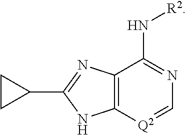

In further embodiments, provided are compounds having a structure represented by a formula:

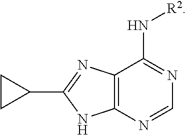

In further embodiments, provided are compounds having a structure represented by a formula:

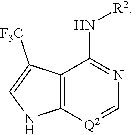

In further embodiments, provided are compounds having a structure represented by a formula:

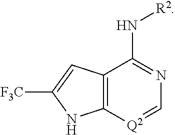

In further embodiments, provided are compounds having a structure represented by a formula:

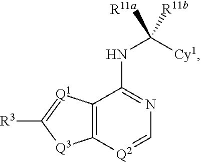

wherein each of R^{11a} and R^{11b} is independently selected from hydrogen, C1-C5 alkyl, and C1-C5 hydroxyalkyl; or wherein each of R^{11a} and R^{11b} together comprise a 3-membered cycloalkyl; and wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, provided are compounds having a structure represented by a formula:

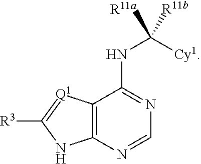

In further embodiments, provided are compounds having a structure represented by a formula selected from:

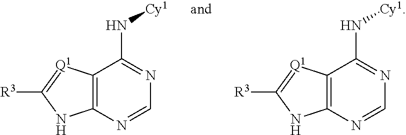

In further embodiments, provided are compounds having a structure represented by a formula:

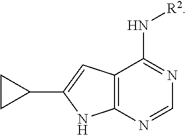

In further embodiments, provided are compounds having a structure represented by a formula:

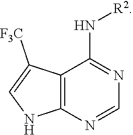

In further embodiments, provided are compounds having a structure represented by a formula:

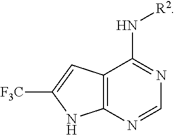

In some embodiments, provided are compounds having a structure represented by a formula:

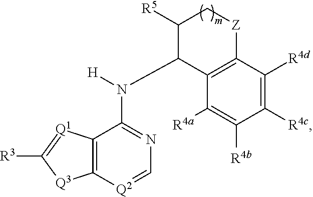

wherein m is 0 or 1; wherein Q¹ is N or CH and R³ is a 3- to 6-membered cycloalkyl, C1-C6 haloalkyl, C1-C6 haloalkoxy, or C1-C6 halohydroxyalkyl; wherein Z is CR^{13a}R^{13b} or O; wherein each of R^{13a} and R^{13b}, when present, is independently selected from hydrogen, halogen, —OH, and C1-C4 alkoxy, or wherein each of R^{13a} and R^{13b}, when present, together comprise ═O; wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R⁵ is selected from —O(CH₂)ₙCy², —NR¹²(CH₂)ₙCy², and Cy².

In some embodiments, provided are compounds having a structure represented by a formula selected from:

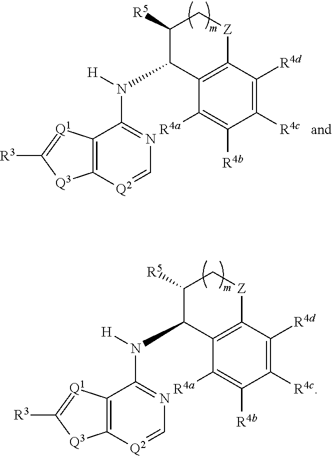

In some embodiments, provided are compounds having a structure represented by a formula selected from:

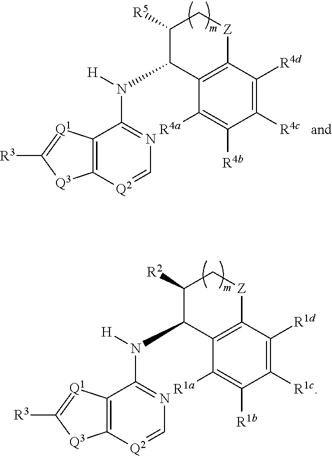

In some embodiments, provided is a compound having a structure:

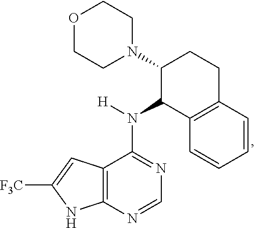

or a pharmaceutically acceptable salt thereof.

In some embodiments, provided is a compound having a structure represented by a formula:

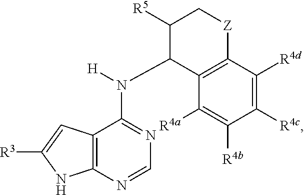

or a pharmaceutically acceptable salt thereof.

In some embodiments, provided is a compound having a structure represented by a formula:

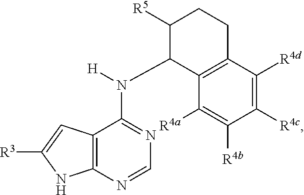

or a pharmaceutically acceptable salt thereof.

In some embodiments, provided is a compound having a structure represented by a formula selected from:

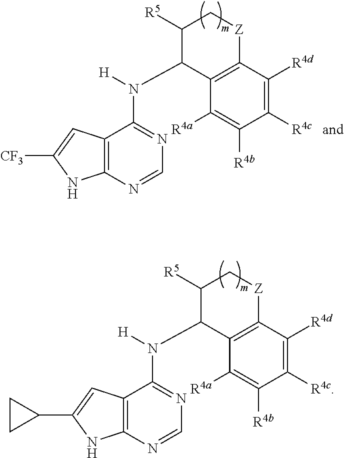

or a pharmaceutically acceptable salt thereof.

In some embodiments, provided is a compound having a structure represented by a formula selected from:

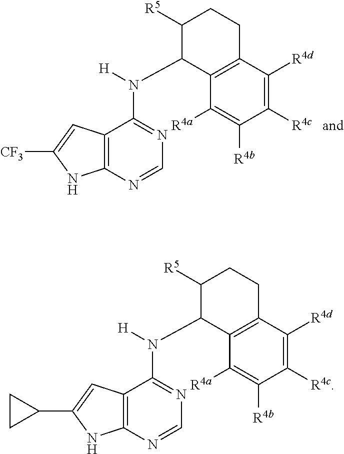

or a pharmaceutically acceptable salt thereof.

In some embodiments, the compound is selected from:

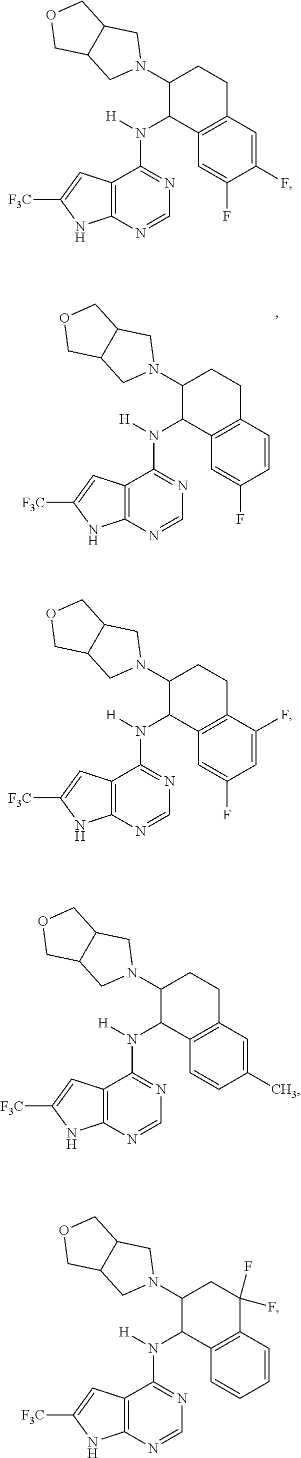 [SMILES: Fc1cc2c(cc1F)C(Nc1ncnc3[nH]c(C(F)(F)F)cc13)C(N1CC3COCC3C1)CC2 ; Fc1ccc2c(c1)C(Nc1ncnc3[nH]c(C(F)(F)F)cc13)C(N1CC3COCC3C1)CC2 ; Fc1cc(F)c2c(c1)C(Nc1ncnc3[nH]c(C(F)(F)F)cc13)C(N1CC3COCC3C1)CC2 ; Cc1ccc2c(c1)CCC(N1CC3COCC3C1)C2Nc1ncnc2[nH]c(C(F)(F)F)cc12 ; FC(F)(F)c1cc2c(NC3c4ccccc4C(F)(F)CC3N3CC4COCC4C3)ncnc2[nH]1]

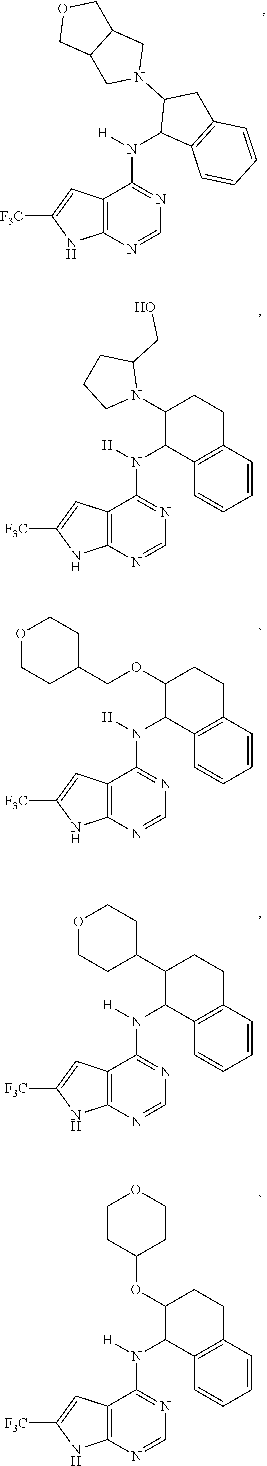 [SMILES: FC(F)(F)c1cc2c(NC3c4ccccc4CC3N3CC4COCC4C3)ncnc2[nH]1 ; OCC1CCCN1C1CCc2ccccc2C1Nc1ncnc2[nH]c(C(F)(F)F)cc12 ; FC(F)(F)c1cc2c(NC3c4ccccc4CCC3OCC3CCOCC3)ncnc2[nH]1 ; FC(F)(F)c1cc2c(NC3c4ccccc4CCC3C3CCOCC3)ncnc2[nH]1 ; FC(F)(F)c1cc2c(NC3c4ccccc4CCC3OC3CCOCC3)ncnc2[nH]1]

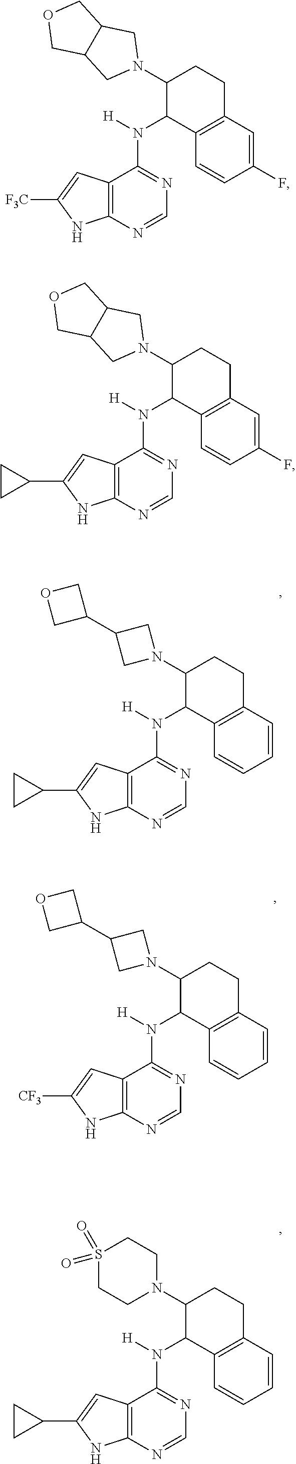 [SMILES: Fc1ccc2c(c1)CCC(N1CC3COCC3C1)C2Nc1ncnc2[nH]c(C(F)(F)F)cc12 ; Fc1ccc2c(c1)CCC(N1CC3COCC3C1)C2Nc1ncnc2[nH]c(C3CC3)cc12 ; c1ccc2c(c1)CCC(N1CC(C3COC3)C1)C2Nc1ncnc2[nH]c(C3CC3)cc12 ; FC(F)(F)c1cc2c(NC3c4ccccc4CCC3N3CC(C4COC4)C3)ncnc2[nH]1 ; O=S1(=O)CCN(C2CCc3ccccc3C2Nc2ncnc3[nH]c(C4CC4)cc23)CC1]

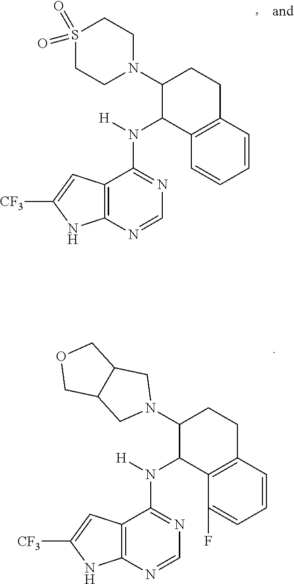 [SMILES: O=S1(=O)CCN(C2CCc3ccccc3C2Nc2ncnc3[nH]c(C(F)(F)F)cc23)CC1 ; Fc1cccc2c1C(Nc1ncnc3[nH]c(C(F)(F)F)cc13)C(N1CC3COCC3C1)CC2]

In some embodiments, the compound is selected from:

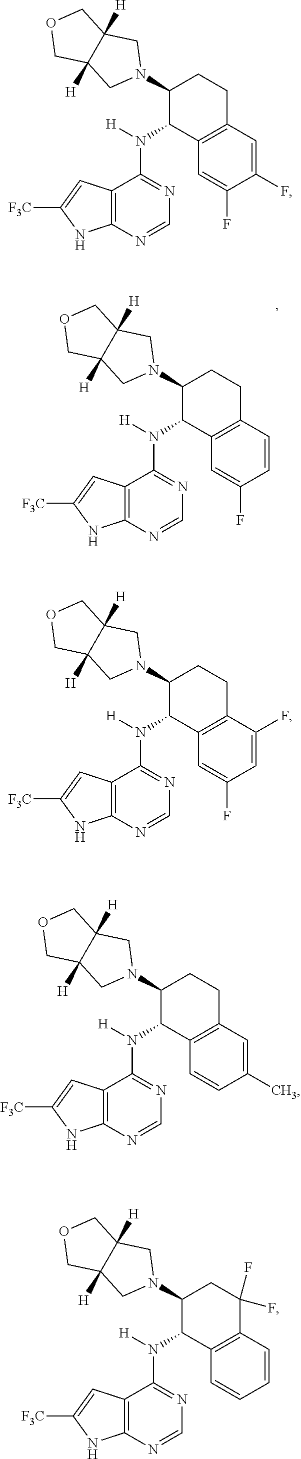 [SMILES: Fc1cc2c(cc1F)[C@H](Nc1ncnc3[nH]c(C(F)(F)F)cc13)[C@@H](N1C[C@H]3COC[C@H]3C1)CC2 ; Fc1ccc2c(c1)[C@H](Nc1ncnc3[nH]c(C(F)(F)F)cc13)[C@@H](N1C[C@H]3COC[C@H]3C1)CC2 ; Fc1cc(F)c2c(c1)[C@H](Nc1ncnc3[nH]c(C(F)(F)F)cc13)[C@@H](N1C[C@H]3COC[C@H]3C1)CC2 ; Cc1ccc2c(c1)CC[C@H](N1C[C@H]3COC[C@H]3C1)[C@H]2Nc1ncnc2[nH]c(C(F)(F)F)cc12 ; FC(F)(F)c1cc2c(N[C@H]3c4ccccc4C(F)(F)C[C@@H]3N3C[C@H]4COC[C@H]4C3)ncnc2[nH]1]

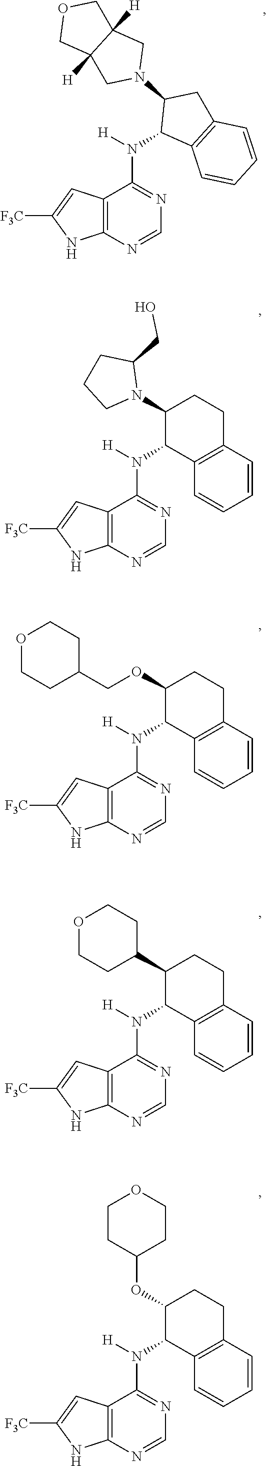 [SMILES: FC(F)(F)c1cc2c(N[C@H]3c4ccccc4C[C@@H]3N3C[C@H]4COC[C@H]4C3)ncnc2[nH]1 ; OC[C@@H]1CCCN1[C@H]1CCc2ccccc2[C@@H]1Nc1ncnc2[nH]c(C(F)(F)F)cc12 ; FC(F)(F)c1cc2c(N[C@H]3c4ccccc4CC[C@@H]3OCC3CCOCC3)ncnc2[nH]1 ; FC(F)(F)c1cc2c(N[C@H]3c4ccccc4CC[C@@H]3C3CCOCC3)ncnc2[nH]1 ; FC(F)(F)c1cc2c(N[C@H]3c4ccccc4CC[C@H]3OC3CCOCC3)ncnc2[nH]1]

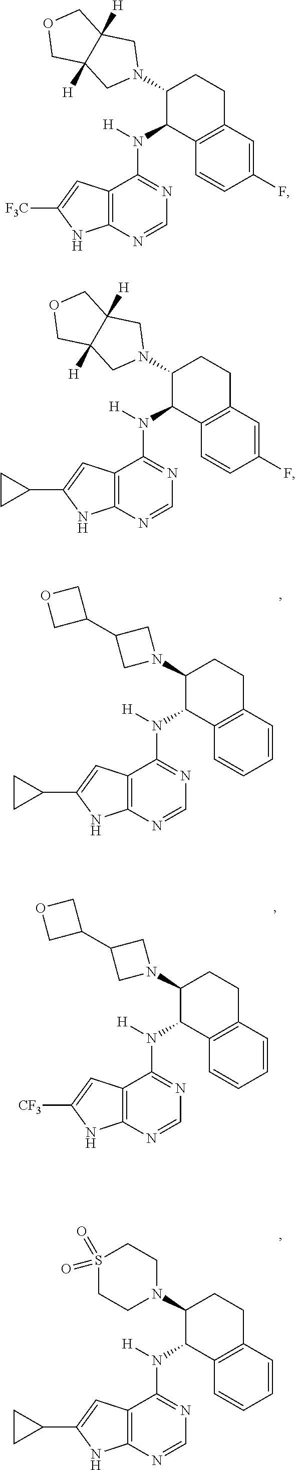 [SMILES: Fc1ccc2c(c1)CC[C@@H](N1C[C@H]3COC[C@H]3C1)[C@@H]2Nc1ncnc2[nH]c(C(F)(F)F)cc12 ; Fc1ccc2c(c1)CC[C@@H](N1C[C@H]3COC[C@H]3C1)[C@@H]2Nc1ncnc2[nH]c(C3CC3)cc12 ; c1ccc2c(c1)CC[C@H](N1CC(C3COC3)C1)[C@H]2Nc1ncnc2[nH]c(C3CC3)cc12 ; FC(F)(F)c1cc2c(N[C@H]3c4ccccc4CC[C@@H]3N3CC(C4COC4)C3)ncnc2[nH]1 ; O=S1(=O)CCN([C@H]2CCc3ccccc3[C@@H]2Nc2ncnc3[nH]c(C4CC4)cc23)CC1]

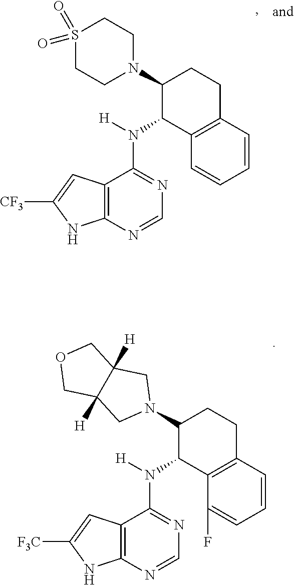

In some embodiments, the compound is selected from:

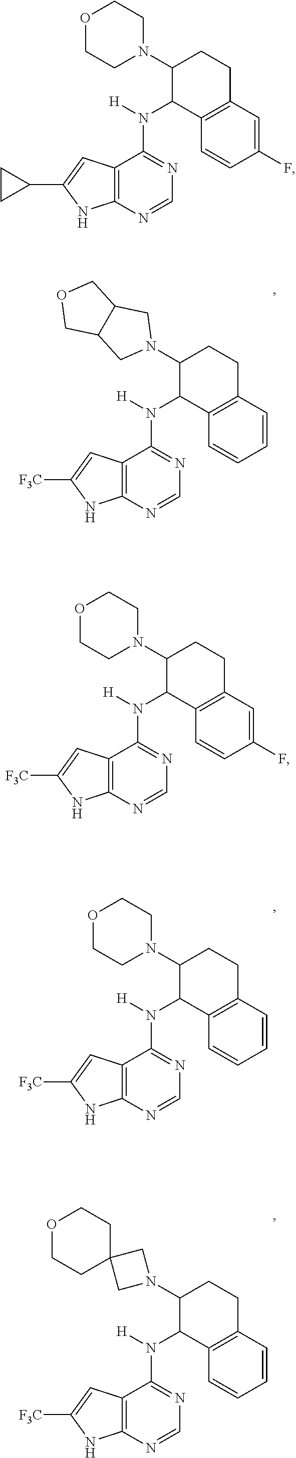 [SMILES: Fc1ccc2c(c1)CCC(N1CCOCC1)C2Nc1ncnc2[nH]c(C3CC3)cc12 ; FC(F)(F)c1cc2c(NC3c4ccccc4CCC3N3CC4COCC4C3)ncnc2[nH]1 ; Fc1ccc2c(c1)CCC(N1CCOCC1)C2Nc1ncnc2[nH]c(C(F)(F)F)cc12 ; FC(F)(F)c1cc2c(NC3c4ccccc4CCC3N3CCOCC3)ncnc2[nH]1 ; FC(F)(F)c1cc2c(NC3c4ccccc4CCC3N3CC4(CCOCC4)C3)ncnc2[nH]1]

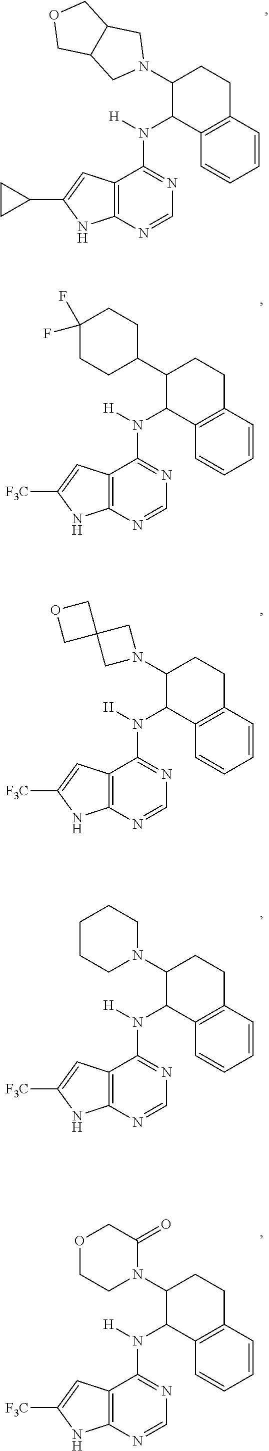 [SMILES: c1ccc2c(c1)CCC(N1CC3COCC3C1)C2Nc1ncnc2[nH]c(C3CC3)cc12 ; FC1(F)CCC(C2CCc3ccccc3C2Nc2ncnc3[nH]c(C(F)(F)F)cc23)CC1 ; FC(F)(F)c1cc2c(NC3c4ccccc4CCC3N3CC4(COC4)C3)ncnc2[nH]1 ; FC(F)(F)c1cc2c(NC3c4ccccc4CCC3N3CCCCC3)ncnc2[nH]1 ; O=C1COCCN1C1CCc2ccccc2C1Nc1ncnc2[nH]c(C(F)(F)F)cc12]

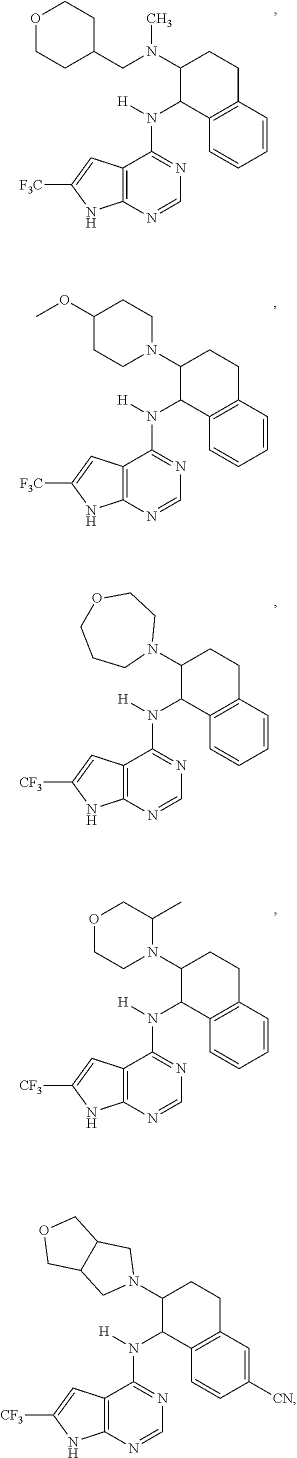 [SMILES: CN(CC1CCOCC1)C1CCc2ccccc2C1Nc1ncnc2[nH]c(C(F)(F)F)cc12 ; COC1CCN(C2CCc3ccccc3C2Nc2ncnc3[nH]c(C(F)(F)F)cc23)CC1 ; FC(F)(F)c1cc2c(NC3c4ccccc4CCC3N3CCCOCC3)ncnc2[nH]1 ; CC1COCCN1C1CCc2ccccc2C1Nc1ncnc2[nH]c(C(F)(F)F)cc12 ; N#Cc1ccc2c(c1)CCC(N1CC3COCC3C1)C2Nc1ncnc2[nH]c(C(F)(F)F)cc12]

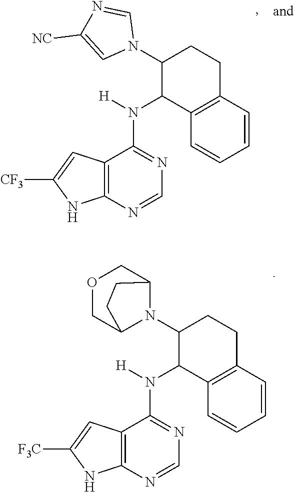 [SMILES: N#Cc1cn(C2CCc3ccccc3C2Nc2ncnc3[nH]c(C(F)(F)F)cc23)cn1 ; FC(F)(F)c1cc2c(NC3c4ccccc4CCC3N3C4CCC3COC4)ncnc2[nH]1]

In some embodiments, the compound is selected from:

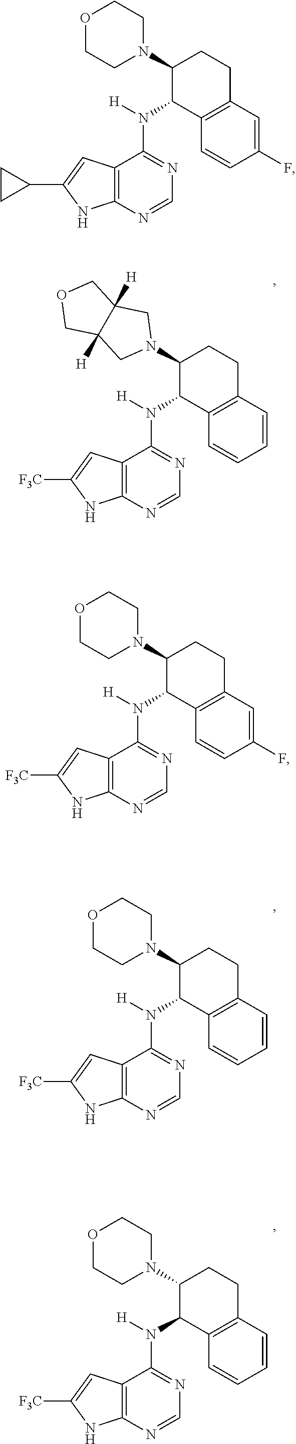 [SMILES: Fc1ccc2c(c1)CC[C@H](N1CCOCC1)[C@H]2Nc1ncnc2[nH]c(C3CC3)cc12 ; FC(F)(F)c1cc2c(N[C@H]3c4ccccc4CC[C@@H]3N3C[C@H]4COC[C@H]4C3)ncnc2[nH]1 ; Fc1ccc2c(c1)CC[C@H](N1CCOCC1)[C@H]2Nc1ncnc2[nH]c(C(F)(F)F)cc12 ; FC(F)(F)c1cc2c(N[C@H]3c4ccccc4CC[C@@H]3N3CCOCC3)ncnc2[nH]1 ; FC(F)(F)c1cc2c(N[C@@H]3c4ccccc4CC[C@H]3N3CCOCC3)ncnc2[nH]1]

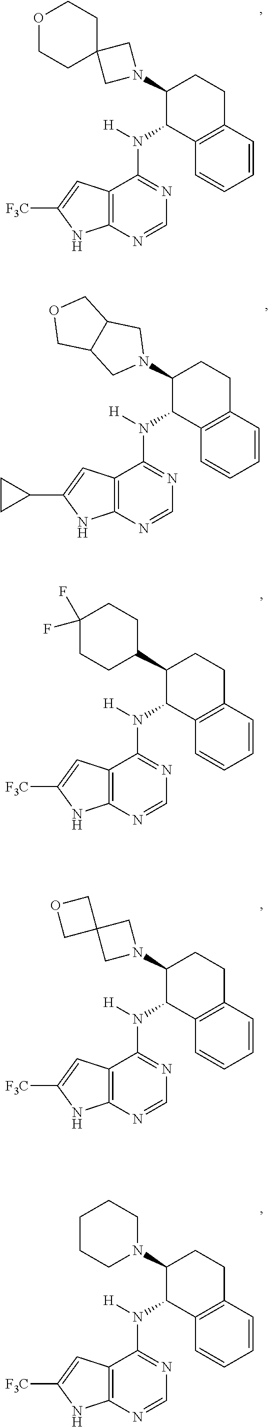 [SMILES: FC(F)(F)c1cc2c(N[C@H]3c4ccccc4CC[C@@H]3N3CC4(CCOCC4)C3)ncnc2[nH]1 ; c1ccc2c(c1)CC[C@H](N1CC3COCC3C1)[C@H]2Nc1ncnc2[nH]c(C3CC3)cc12 ; FC1(F)CCC([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CC4(COC4)C3)ncnc2[nH]1 ; FC(F)(F)c1cc2c(N[C@H]3c4ccccc4CC[C@@H]3N3CCCCC3)ncnc2[nH]1]

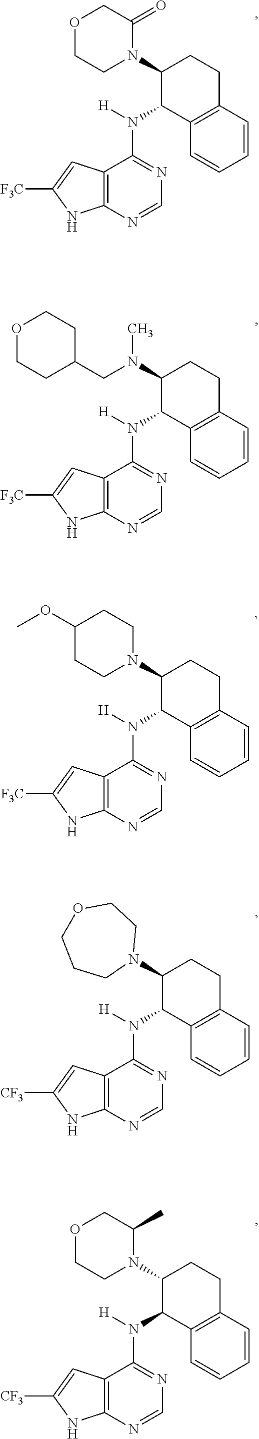 [SMILES: O=C1COCCN1[C@H]1CCc2ccccc2[C@@H]1Nc1ncnc2[nH]c(C(F)(F)F)cc12 ; CN(CC1CCOCC1)[C@H]1CCc2ccccc2[C@@H]1Nc1ncnc2[nH]c(C(F)(F)F)cc12 ; COC1CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CCCOCC3)ncnc2[nH]1 ; C[C@@H]1COCCN1[C@@H]1CCc2ccccc2[C@H]1Nc1ncnc2[nH]c(C(F)(F)F)cc12]

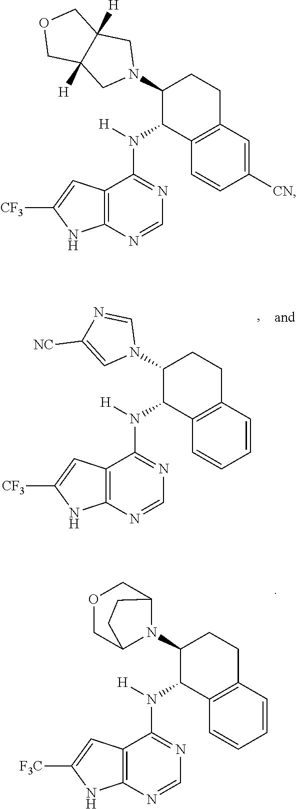 [SMILES: N#Cc1ccc2c(c1)CC[C@H](N1C[C@H]3COC[C@H]3C1)[C@H]2Nc1ncnc2[nH]c(C(F)(F)F)cc12 ; N#Cc1cn([C@@H]2CCc3ccccc3[C@@H]2Nc2ncnc3[nH]c(C(F)(F)F)cc23)cn1 ; FC(F)(F)c1cc2c(N[C@H]3c4ccccc4CC[C@@H]3N3C4CCC3COC4)ncnc2[nH]1]

In some embodiments, Q¹ is CH, Q² is N, and Q³ is NH.

In further embodiments, Q¹ is N and R³ is a 3- to 6-membered cycloalkyl. In still further embodiments, Q¹ is N and R³ is a 3- to 4-membered cycloalkyl.

In further embodiments, Q¹ is CR¹ and R³ is hydrogen.

In further embodiments, Cy¹, when present, is a structure represented by a formula selected from:

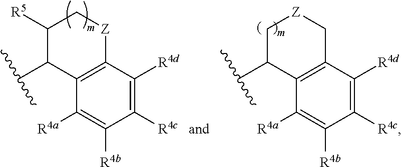

wherein m is 0 or 1; wherein Z is O, CR^{13a}R^{13b}, or NR¹⁴; wherein each of R^{13a} and R^{13b}, when present, is independently selected from hydrogen, halogen, —OH, and C1-C4 alkoxy, or wherein each of R^{13a} and R^{13b}, when present, together comprise ═O; and wherein R¹⁴, when present, is selected from —C(O)(C1-C4 alkyl), C1-C4 alkyl, and C2-C4 alkenyl; wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R⁵, when present, is selected from hydrogen, —O(CH₂)ₙCy², —NR¹²(CH₂)ₙCy², and Cy².

In further embodiments, Cy¹, when present, is a structure represented by a formula selected from:

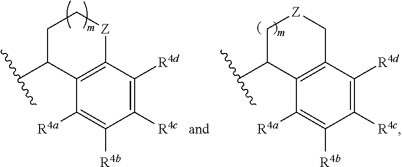

wherein Z is O or CH₂; wherein n is 0 or 1; and wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, Cy¹, when present, is a structure represented by a formula selected from:

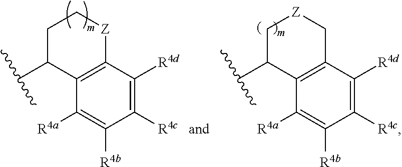

wherein Z is O, CH₂, or NR¹⁴; wherein R¹⁴, when present, is selected from —C(O)(C1-C4 alkyl), C1-C4 alkyl, and C2-C4 alkenyl; wherein n is 0 or 1; and wherein each of R^{4a}, R^{4b}, R^{4c}, and R^{4d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, the compound has a structure represented by a formula:

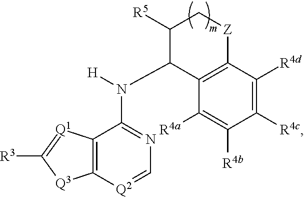

wherein Z is O, CH₂, or NR¹⁴; wherein R¹⁴, when present, is selected from —C(O)(C1-C4 alkyl), C1-C4 alkyl, and C2-C4 alkenyl; wherein n is 0 or 1; wherein each of R^{4a}, R^{4b}, R^{4c}, and R^{4d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R⁵ is selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, the compound has a structure represented by a formula selected from:

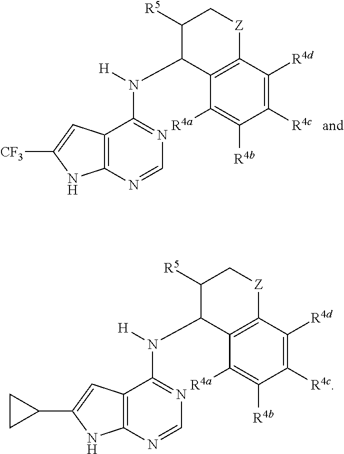

In further embodiments, the compound has a structure represented by a formula:

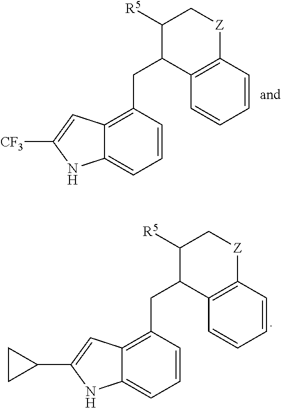

In further embodiments, the compound has a structure selected from:

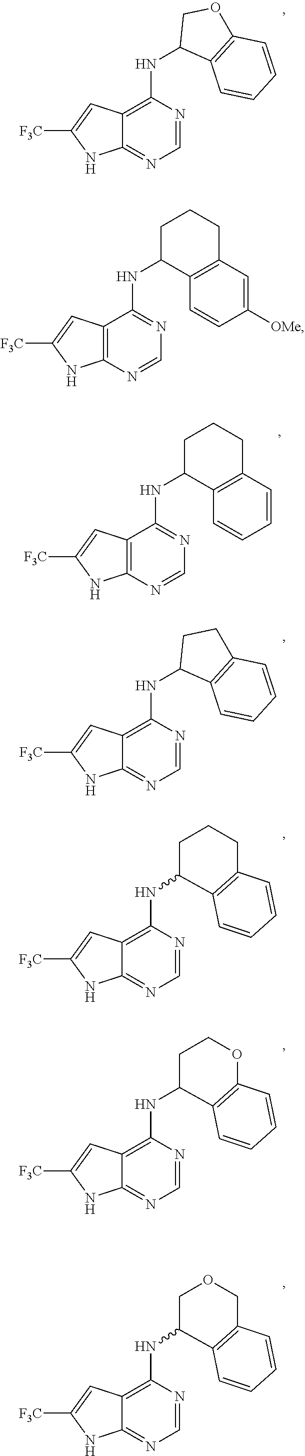

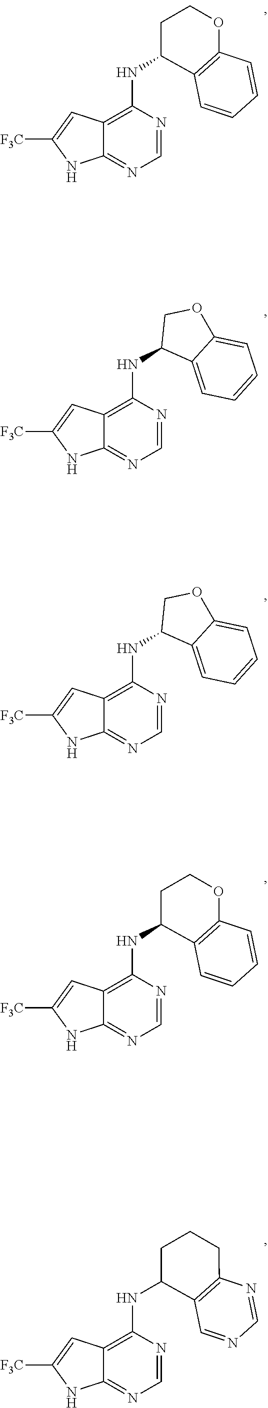 [SMILES: FC(F)(F)c1cc2c(N[C@@H]3CCOc4ccccc43)ncnc2[nH]1 ; FC(F)(F)c1cc2c(N[C@H]3COc4ccccc43)ncnc2[nH]1 ; FC(F)(F)c1cc2c(N[C@@H]3COc4ccccc43)ncnc2[nH]1 ; FC(F)(F)c1cc2c(N[C@H]3CCOc4ccccc43)ncnc2[nH]1 ; FC(F)(F)c1cc2c(NC3CCCc4ncncc43)ncnc2[nH]1]

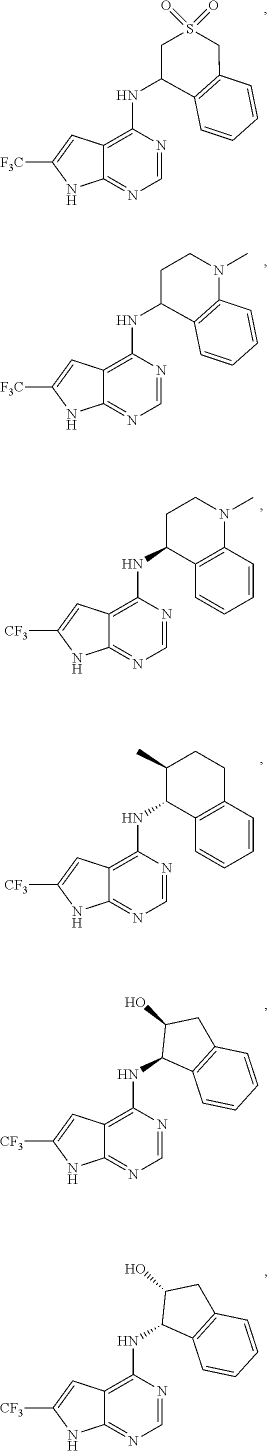 [SMILES: O=S1(=O)Cc2ccccc2C(Nc2ncnc3[nH]c(C(F)(F)F)cc23)C1 ; CN1CCC(Nc2ncnc3[nH]c(C(F)(F)F)cc23)c2ccccc21 ; CN1CC[C@H](Nc2ncnc3[nH]c(C(F)(F)F)cc23)c2ccccc21 ; C[C@H]1CCc2ccccc2[C@@H]1Nc1ncnc2[nH]c(C(F)(F)F)cc12 ; O[C@H]1Cc2ccccc2[C@H]1Nc1ncnc2[nH]c(C(F)(F)F)cc12 ; O[C@@H]1Cc2ccccc2[C@@H]1Nc1ncnc2[nH]c(C(F)(F)F)cc12]

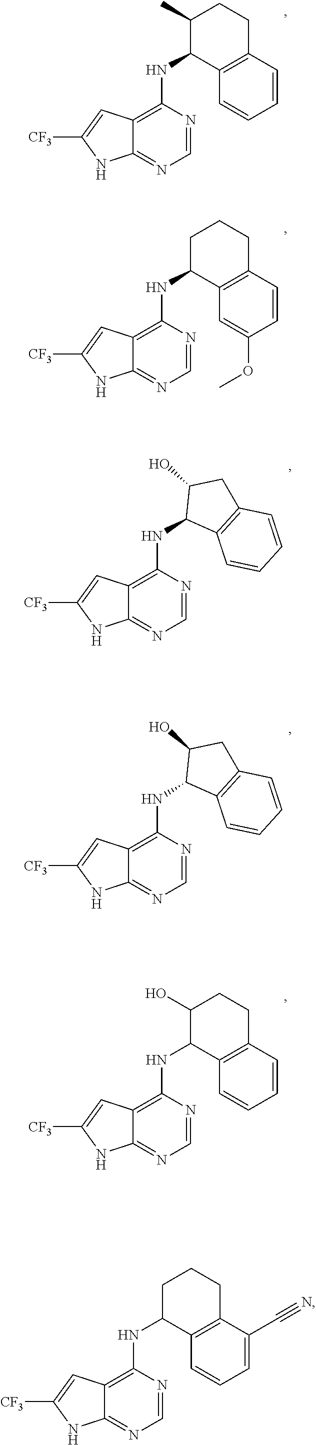 [SMILES: C[C@H]1CCc2ccccc2[C@H]1Nc1ncnc2[nH]c(C(F)(F)F)cc12 ; COc1ccc2c(c1)[C@@H](Nc1ncnc3[nH]c(C(F)(F)F)cc13)CCC2 ; O[C@@H]1Cc2ccccc2[C@H]1Nc1ncnc2[nH]c(C(F)(F)F)cc12 ; O[C@H]1Cc2ccccc2[C@@H]1Nc1ncnc2[nH]c(C(F)(F)F)cc12 ; OC1CCc2ccccc2C1Nc1ncnc2[nH]c(C(F)(F)F)cc12 ; N#Cc1cccc2c1CCCC2Nc1ncnc2[nH]c(C(F)(F)F)cc12]

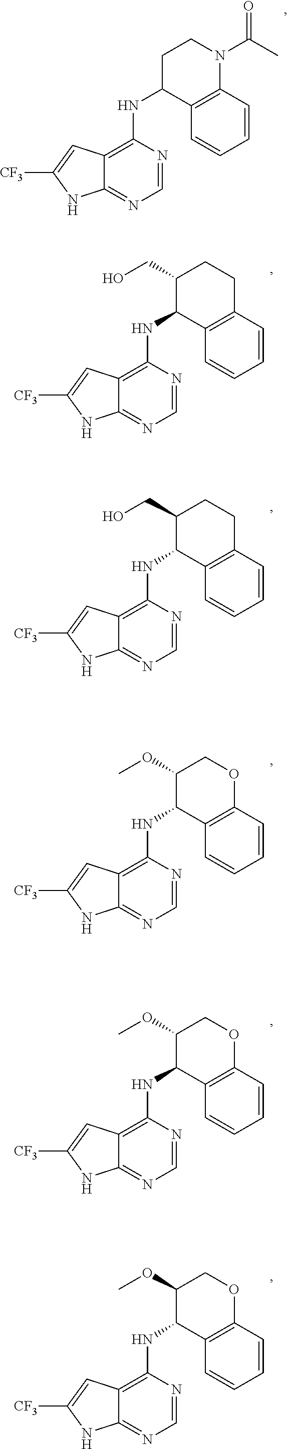 [SMILES: CC(=O)N1CCC(Nc2ncnc3[nH]c(C(F)(F)F)cc23)c2ccccc21 ; OC[C@@H]1CCc2ccccc2[C@H]1Nc1ncnc2[nH]c(C(F)(F)F)cc12 ; OC[C@H]1CCc2ccccc2[C@@H]1Nc1ncnc2[nH]c(C(F)(F)F)cc12 ; CO[C@@H]1COc2ccccc2[C@@H]1Nc1ncnc2[nH]c(C(F)(F)F)cc12 ; CO[C@@H]1COc2ccccc2[C@H]1Nc1ncnc2[nH]c(C(F)(F)F)cc12 ; CO[C@H]1COc2ccccc2[C@@H]1Nc1ncnc2[nH]c(C(F)(F)F)cc12]

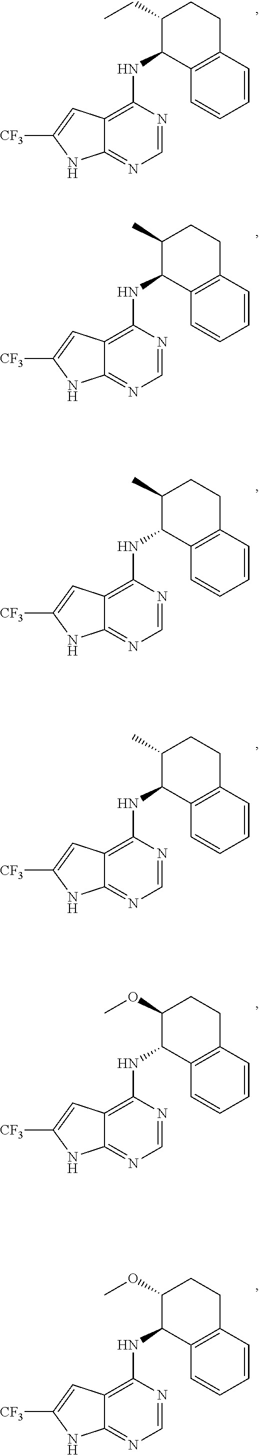 [SMILES: CC[C@@H]1CCc2ccccc2[C@H]1Nc1ncnc2[nH]c(C(F)(F)F)cc12 ; C[C@H]1CCc2ccccc2[C@H]1Nc1ncnc2[nH]c(C(F)(F)F)cc12 ; C[C@H]1CCc2ccccc2[C@@H]1Nc1ncnc2[nH]c(C(F)(F)F)cc12 ; C[C@@H]1CCc2ccccc2[C@H]1Nc1ncnc2[nH]c(C(F)(F)F)cc12 ; CO[C@H]1CCc2ccccc2[C@@H]1Nc1ncnc2[nH]c(C(F)(F)F)cc12 ; CO[C@@H]1CCc2ccccc2[C@H]1Nc1ncnc2[nH]c(C(F)(F)F)cc12]

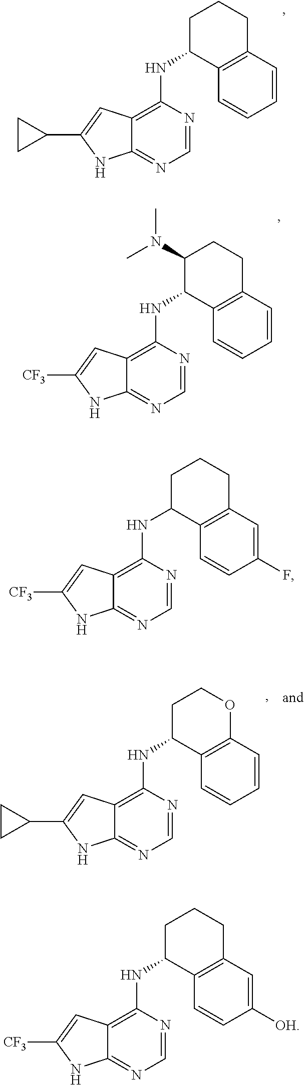

In further embodiments, the compound has a structure:

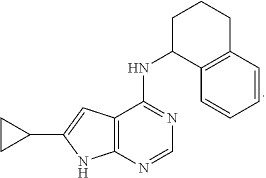 [SMILES: c1ccc2c(c1)CCCC2Nc1ncnc2[nH]c(C3CC3)cc12]

In further embodiments, the compound has a structure selected from:

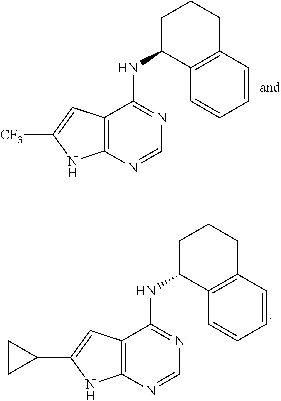 [SMILES: FC(F)(F)c1cc2c(N[C@H]3CCCc4ccccc43)ncnc2[nH]1 ; c1ccc2c(c1)CCC[C@H]2Nc1ncnc2[nH]c(C3CC3)cc12]

In further embodiments, the compound has a structure:

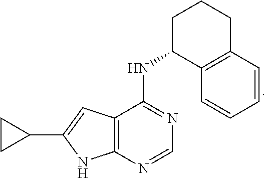 [SMILES: c1ccc2c(c1)CCC[C@H]2Nc1ncnc2[nH]c(C3CC3)cc12]

In some embodiments, the compound has a structure represented by a formula:

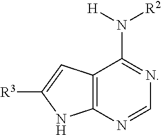

In some embodiments, the compound has a structure represented by a formula:

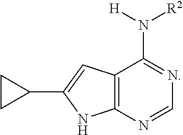

In some embodiments, the compound has a structure represented by a formula:

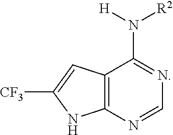

In some embodiments, the compound has a structure represented by a formula:

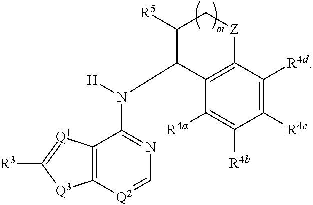

wherein m is 0 or 1; wherein Q¹ is N or CH and R³ is a 3- to 6-membered cycloalkyl, C1-C6 haloalkyl, C1-C6 haloalkoxy, or C1-C6 halohydroxyalkyl; wherein Z is CR^{13a}R^{13b} or O; wherein each of R^{13a} and R^{13b}, when present, is independently selected from hydrogen, halogen, —OH, and C1-C4 alkoxy, or wherein each of R^{13a} and R^{13b}, when present, together comprise ═O; wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R⁵ is selected from —O(CH₂)ₙCy², —NR¹²(CH₂)ₙCy², and Cy².

In some embodiments, the compound is selected from:

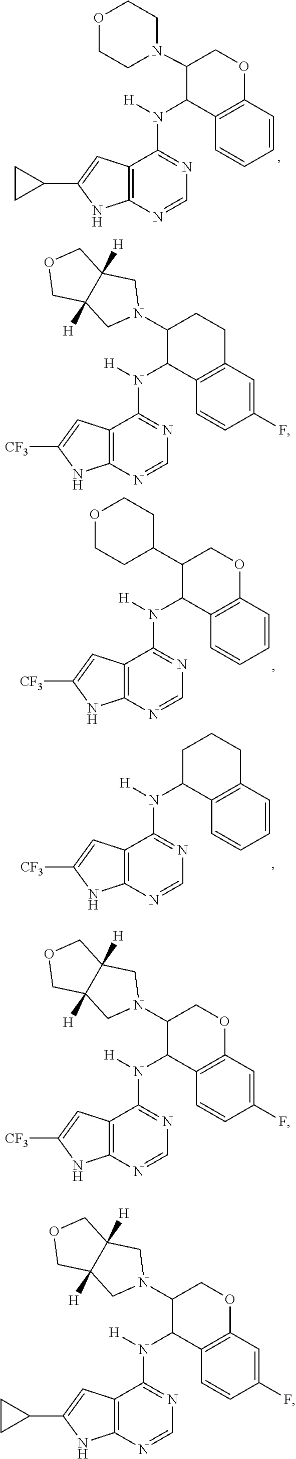

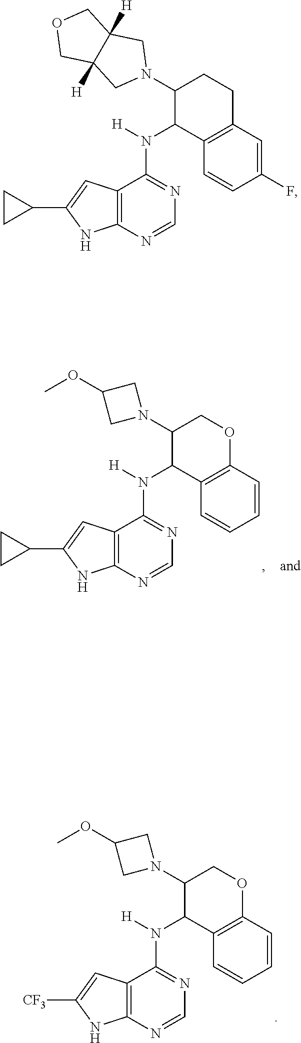 [SMILES: Fc1ccc2c(c1)CCC(N1C[C@H]3COC[C@H]3C1)C2Nc1ncnc2[nH]c(C3CC3)cc12 ; COC1CN(C2COc3ccccc3C2Nc2ncnc3[nH]c(C4CC4)cc23)C1 ; COC1CN(C2COc3ccccc3C2Nc2ncnc3[nH]c(C(F)(F)F)cc23)C1]

In some embodiments, the compound is selected from:

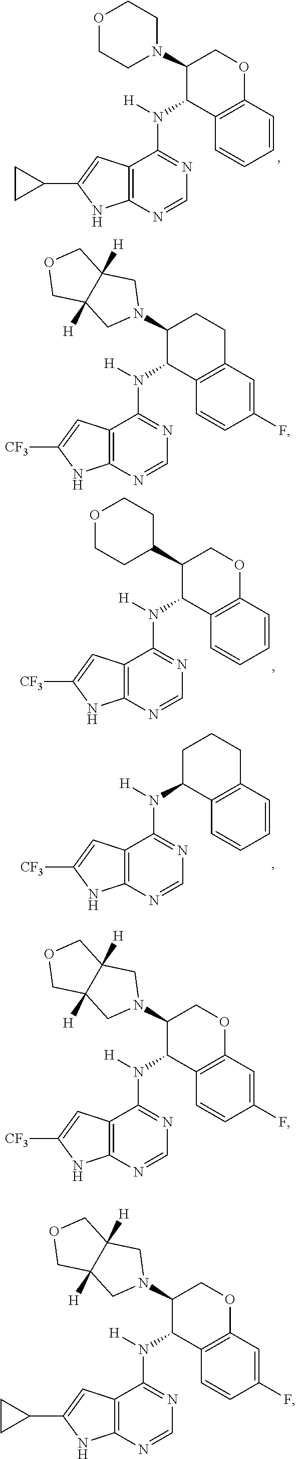

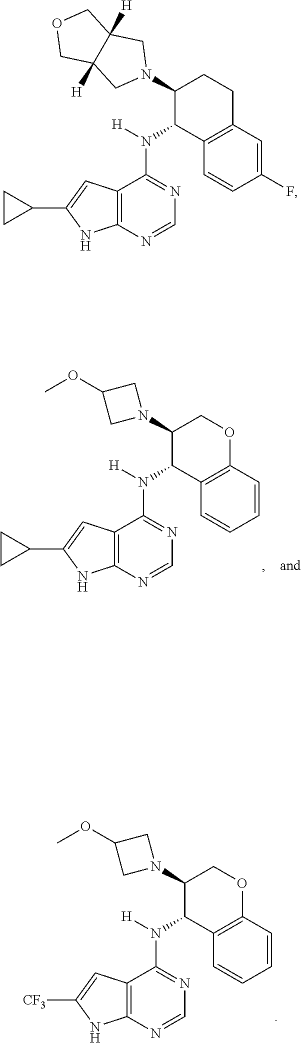 [SMILES: Fc1ccc2c(c1)CC[C@H](N1C[C@H]3COC[C@H]3C1)[C@H]2Nc1ncnc2[nH]c(C3CC3)cc12 ; COC1CN([C@H]2COc3ccccc3[C@@H]2Nc2ncnc3[nH]c(C4CC4)cc23)C1 ; COC1CN([C@H]2COc3ccccc3[C@@H]2Nc2ncnc3[nH]c(C(F)(F)F)cc23)C1]

In some embodiments, the compound is selected from:

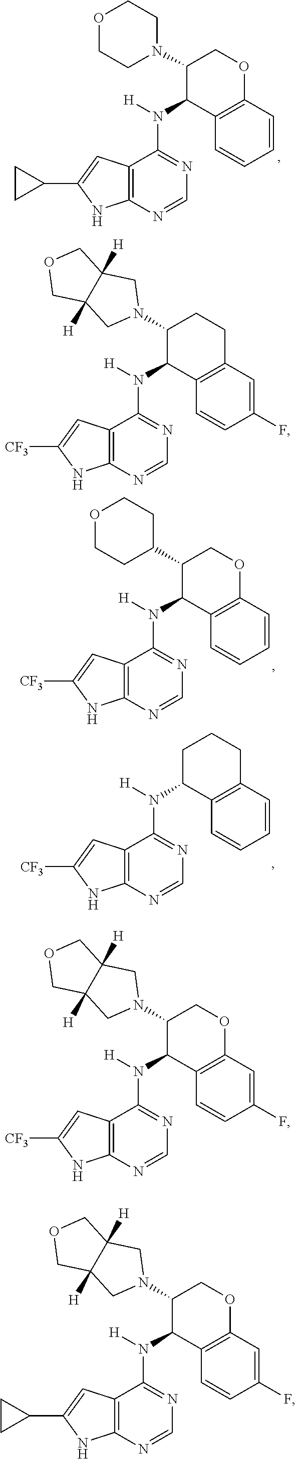

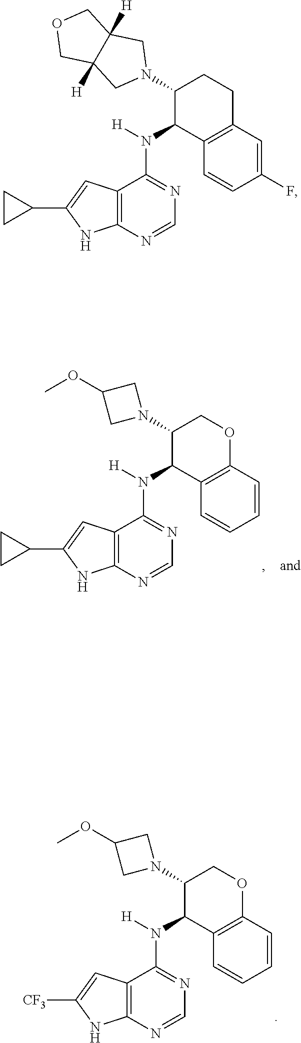 [SMILES: Fc1ccc2c(c1)CC[C@@H](N1C[C@H]3COC[C@H]3C1)[C@@H]2Nc1ncnc2[nH]c(C3CC3)cc12 ; COC1CN([C@@H]2COc3ccccc3[C@H]2Nc2ncnc3[nH]c(C4CC4)cc23)C1 ; COC1CN([C@@H]2COc3ccccc3[C@H]2Nc2ncnc3[nH]c(C(F)(F)F)cc23)C1]

Thus, in some embodiments, n is 0 or 1. In further embodiments, n is 0. In still further embodiments, n is 1.

Thus, in some embodiments, m is 0 or 1. In further embodiments, m is 0. In still further embodiments, m is 1.

In some embodiments, n, when present, is 0, 1, or 2. In further embodiments, n, when present, is 0 or 1. In still further embodiments, n, when present, is 1 or 2. In yet further embodiments, n, when present, is 0 or 2. In even further embodiments, n, when present, is 0. In still further embodiments, n, when present, is 0. In yet further embodiments, n, when present, is 1. In even further embodiments, n, when present, is 2.

Specific examples of compounds are provided in the EXAMPLE COMPOUNDS section and are included herein. Pharmaceutically acceptable salts as well as the neutral forms of these compounds are also included.

a. Q¹ Groups

In some embodiments, Q¹ is CH or N. In further embodiments, Q¹ is N. In still further embodiments, Q¹ is CH.

In some embodiments, Q¹ is CR¹.

b. Q² Groups

In some embodiments, Q² is CH or N. In further embodiments, Q² is CH. In still further embodiments, Q² is N.

c. Q³ Groups

In some embodiments, Q³ is CH₂ or NH. In further embodiments, Q³ is CH₂. In still further embodiments, Q³ is NH.

d. Z Groups

In some embodiments, Z is CR^{13a}R^{13b}, NR¹⁴, or O. In further embodiments, Z is CR^{13a}R^{13b} or NR¹⁴. In still further embodiments, Z is NR¹⁴ or O.

In some embodiments, Z is CR^{13a}R^{13b} or O. In further embodiments, Z is CR^{13a}R^{13b} In still further embodiments, Z is CH₂. In yet further embodiments, Z is O.

In some embodiments, Z is NR¹⁴.

e. R¹ Groups

In some embodiments, R¹ is C1-C6 haloalkyl, C1-C6 haloalkoxy, C1-C6 halohydroxy, or a structure represented by a formula:

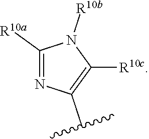

In further embodiments, R¹ is C1-C3 haloalkyl, C1-C3 haloalkoxy, C1-C3 halohydroxy, or a structure represented by a formula:

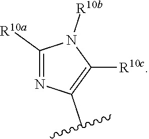

In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —CH(OCH₃)CF₃, —CH(OCH₃)CHF₂, —CH(OCH₃)CH₂F, —CH(OCH₃)CCl₃, —CH(OCH₃)CHCl₂, —CH(OCH₃)CH₂Cl, —CH(OH)CF₃, —CH(OH)CHF₂, —CH(OH)CH₂F, —CH(OH)CCl₃, —CH(OH)CHCl₂, —CH(OH)CH₂Cl, or a structure represented by a formula:

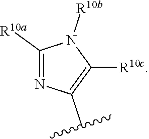

In yet further embodiments, R¹ is —CF₃, —CHF₂, —CH₂F, —CCl₃, —CHCl₂, —CH₂Cl, or a structure represented by a formula:

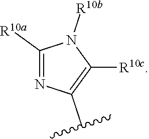

In various embodiments, R¹ is a structure represented by a formula:

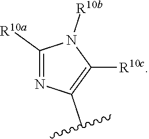

In various embodiments, R¹ is C1-C6 haloalkyl, C1-C6 haloalkoxy, or C1-C6 halohydroxy. In further embodiments, R¹ is C1-C3 haloalkyl, C1-C3 haloalkoxy, or C1-C3 halohydroxy. In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —CH(OCH₃)CF₃, —CH(OCH₃)CHF₂, —CH(OCH₃)CH₂F, —CH(OCH₃)CCl₃, —CH(OCH₃)CHCl₂, —CH(OCH₃)CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, R¹ is —CF₃, —CHF₂, —CH₂F, —CCl₃, —CHCl₂, or —CH₂Cl.

In various embodiments, R¹ is C1-C6 haloalkoxy or C1-C6 halohydroxy. In further embodiments, R¹ is —CH(OCH₂CH₃)CF₃, —CH(OCH₂CH₃)CHF₂, —CH(OCH₂CH₃)CH₂F, —CH(OCH₂CH₃)CCl₃, —CH(OCH₂CH₃)CHCl₂, —CH(OCH₂CH₃)CH₂Cl, —CH₂CH(OH)CF₃, —CH₂CH(OH)CHF₂, —CH₂CH(OH)CH₂F, —CH₂CH(OH)CCl₃, —CH₂CH(OH)CHCl₂, or —CH₂CH(OH)CH₂Cl. In still further embodiments, R¹ is —CH(OCH₃)CF₃, —CH(OCH₃)CHF₂, —CH(OCH₃)CH₂F, —CH(OCH₃)CCl₃, —CH(OCH₃)CHCl₂, —CH(OCH₃)CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, —CH(OCH₂CH₃)CF₃ or —CH(OH)CF₃.

In various embodiments, R¹ is C1-C6 haloalkyl. In further embodiments, R¹ is C1-C3 haloalkyl. In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R¹ is —CF₃, —CHF₂, —CH₂F, —CCl₃, —CHCl₂, or —CH₂Cl. In even further embodiments, R¹ is —CF₃ or —CCl₃. In still further embodiments, R¹ is —CF₃.

In some embodiments, R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said (C₁-C₆)alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}.

In various embodiments, R¹ is (C₁-C₆)alkyl, halo(C₁-C₄)alkyl, (C₁-C₄)alkoxy, halo(C₁-C₄)alkoxy, 5- or 6-membered heteroaryl, or phenyl. In further embodiments, R¹ is methyl, ethyl, n-propyl, isopropyl, —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, —CH₂CH₂CH₂Cl, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, —OCH₂CH₂CH₂Cl, 5- or 6-membered heteroaryl, or phenyl. In still further embodiments, R¹ is methyl, ethyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, methoxy, ethoxy, —OCF₃, —OCHF₂, —OCH₂F, —OCH₂CH₂F, —OCCl₃, —OCHCl₂, —OCH₂Cl, —OCH₂CH₂Cl, 5- or 6-membered heteroaryl, or phenyl. In yet further embodiments, R¹ is methyl, —CF₃, —CHF₂, —CH₂F, —CCl₃, —CHCl₂, —CH₂Cl, methoxy, —OCF₃, —OCHF₂, —OCH₂F, —OCCl₃, —OCHCl₂, —OCH₂Cl, 5- or 6-membered heteroaryl, or phenyl.

In various embodiments, R¹ is (C₁-C₆)alkyl, halo(C₁-C₄)alkyl, (C₁-C₄)alkoxy, or halo(C₁-C₄)alkoxy. In further embodiments, R¹ is methyl, ethyl, n-propyl, isopropyl, —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, —CH₂CH₂CH₂Cl, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, or —OCH₂CH₂CH₂Cl. In still further embodiments, R¹ is methyl, ethyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, methoxy, ethoxy, —OCF₃, —OCHF₂, —OCH₂F, —OCH₂CH₂F, —OCCl₃, —OCHCl₂, —OCH₂Cl, or —OCH₂CH₂Cl. In yet further embodiments, R¹ is methyl, —CF₃, —CHF₂, —CH₂F, —CCl₃, —CHCl₂, —CH₂Cl, methoxy, —OCF₃, —OCHF₂, —OCH₂F, —OCCl₃, —OCHCl₂, or —OCH₂Cl.

In various embodiments, R¹ is (C₁-C₆)alkyl or halo(C₁-C₄)alkyl and is optionally and independently substituted with a OR^{a} group. In further embodiments, R¹ is (C₁-C₆)alkyl or halo(C₁-C₄)alkyl and is unsubstituted.

In various embodiments, R¹ is 5- or 6-membered heteroaryl, or phenyl. Examples of 5- or 6-membered heteroaryls include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, and pyrazinyl. Thus, in various embodiments, R¹ is 5-membered heteroaryl or phenyl. In further embodiments, R¹ is 6-membered heteroaryl or phenyl. In still further embodiments, R¹ is 5-membered heteroaryl. In yet further embodiments, R¹ is 6-membered heteroaryl. In even further embodiments, R¹ is phenyl.

In various embodiments, R¹ is 5- or 6-membered heteroaryl, or phenyl, and is optionally and independently substituted with 1 to 3 groups independently selected from R^{b}. In further embodiments, R¹ is 5- or 6-membered heteroaryl, or phenyl, and is optionally and independently substituted with 1 to 2 groups independently selected from R^{b}. In still further embodiments, R¹ is 5- or 6-membered heteroaryl, or phenyl, and is optionally monosubstituted with a R^{b} group. In yet further embodiments, R¹ is 5- or 6-membered heteroaryl, or phenyl, and is unsubstituted.

In various embodiments, R¹ is (C₁-C₆)alkyl or (C₁-C₄)alkoxy. In further embodiments, R¹ is methyl, ethyl, n-propyl, isopropyl, methoxy, ethoxy, n-propoxy, or isopropoxy. In still further embodiments, R¹ is methyl, ethyl, methoxy, or ethoxy. In yet further embodiments, R¹ is methyl or methoxy.

In various embodiments, R¹ is halo(C₁-C₄)alkyl or halo(C₁-C₄)alkoxy. In further embodiments, R¹ is —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, —CH₂CH₂CH₂Cl, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, or —OCH₂CH₂CH₂Cl. In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CH₂F, —OCCl₃, —OCHCl₂, —OCH₂Cl, or —OCH₂CH₂Cl. In yet further embodiments, R¹ is —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In various embodiments, R¹ is halo(C₁-C₄)alkyl. In further embodiments, R¹ is —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, or —CH₂CH₂CH₂Cl. In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R¹ is —CF₃, —CHF₂, —CH₂F, —CCl₃, —CHCl₂, or —CH₂Cl.

In various embodiments, R¹ is —CF₃ or —CH₂CF₃. In further embodiments, R¹ is —CH₂CF₃. In still further embodiments, R¹ is —CF₃.

f. R² Groups

In some embodiments, R² is C1-C6 alkyl, —CR^{11a}R^{11b}Cy¹, or Cy¹. In further embodiments, R² is C1-C3 alkyl, —CR^{11a}R^{11b}Cy¹, or Cy¹. In still further embodiments, R² is methyl, ethyl, —CR^{11a}R^{11b}Cy¹, or Cy¹. In yet further embodiments, R² is methyl, —CR^{11a}R^{11b}Cy¹, or Cy¹.

In further embodiments, R² is C1-C6 alkyl. In still further embodiments, R² is methyl, ethyl, n-propyl, isopropyl, n-butyl, isobutyl, sec-butyl, or tert-butyl. In yet further embodiments, R² is methyl, ethyl, n-propyl, or isopropyl. In even further embodiments, R² is methyl or ethyl. In still further embodiments, R² is n-butyl.

In further embodiments, R² is —CR^{11a}R^{11b}Cy¹ or Cy¹. In still further embodiments, R² is —CR^{11a}R^{11b}Cy¹. In yet further embodiments, R² is —CH₂Cy¹, —CH(CH₃)Cy¹, or —C(CH₃)₂Cy¹. In even further embodiments, R² is Cy¹.

In some embodiments, R² is (C₁-C₆)alkyl, 9-membered oxygen-containing fused heterocycle, or 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}.

In various embodiments, R² is (C₁-C₄)alkyl, 9-membered oxygen-containing fused heterocycle, or 9- to 10-membered carbocycle. In further embodiments, R² is methyl, ethyl, n-propyl, isopropyl, 9-membered oxygen-containing fused heterocycle, or 9- to 10-membered carbocycle. In still further embodiments, R² is methyl, ethyl, 9-membered oxygen-containing fused heterocycle, or 9- to 10-membered carbocycle. In yet further embodiments, R² is methyl, 9-membered oxygen-containing fused heterocycle, or 9- to 10-membered carbocycle.

In various embodiments, R² is (C₁-C₆)alkyl optionally substituted with 1 or 2 groups independently selected from R^{c}. In further embodiments, R² is (C₁-C₆)alkyl optionally monosubstituted with a R^{c} group. In still further embodiments, R² is unsubstituted (C₁-C₆)alkyl.

In various embodiments, R² is (C₁-C₆)alkyl. In further embodiments, R² is (C₁-C₄)alkyl. In still further embodiments, R² is methyl, ethyl, n-propyl, or isopropyl. In yet further embodiments, R² is methyl or ethyl. In even further embodiments, R² is ethyl. In still further embodiments, R² is methyl.

In various embodiments, R² is 9-membered oxygen-containing fused heterocycle or 9- to 10-membered carbocycle, and is optionally and independently substituted with 1 to 3 groups independently selected from R^{d}. In further embodiments, R² is 9-membered oxygen-containing fused heterocycle or 9- to 10-membered carbocycle, and is optionally and independently substituted with 1 to 2 groups independently selected from R^{d}. In still further embodiments, R² is 9-membered oxygen-containing fused heterocycle or 9- to 10-membered carbocycle, and is optionally monosubstituted with a R^{d} group. In yet further embodiments, R² is 9-membered oxygen-containing fused heterocycle or 9- to 10-membered carbocycle, and is unsubstituted.

In various embodiments, R² is 9-membered oxygen-containing fused heterocycle or 9- to 10-membered carbocycle. In further embodiments, R² is 9-membered oxygen-containing fused heterocycle. In still further embodiments, R² is 9- to 10-membered carbocycle. In yet further embodiments, R² is 9-membered carbocycle. In even further embodiments, R² is 10-membered carbocycle.

g. R³ Groups

In some embodiments, R³ is a 3- to 6-membered cycloalkyl, C1-C6 haloalkyl, C1-C6 haloalkoxy, or C1-C6 halohydroxyalkyl. In further embodiments, R³ is a 3- to 6-membered cycloalkyl, C1-C4 haloalkyl, C1-C4 haloalkoxy, or C1-C4 halohydroxyalkyl. In still further embodiments, R³ is a 3- to 6-membered cycloalkyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CF₃, —OCH₂CHF₂, —OCH₂CH₂F, —OCCl₃, —OCHCl₂, —OCH₂Cl, —OCH₂CCl₃, —OCH₂CHCl₂, —OCH₂CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, R³ is a 3- to 6-membered cycloalkyl, —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In some embodiments, R³ is C1-C6 haloalkyl, C1-C6 haloalkoxy, or C1-C6 halohydroxyalkyl. In further embodiments, R³ is C1-C4 haloalkyl, C1-C4 haloalkoxy, or C1-C4 halohydroxyalkyl. In still further embodiments, R³ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CF₃, —OCH₂CHF₂, —OCH₂CH₂F, —OCCl₃, —OCHCl₂, —OCH₂Cl, —OCH₂CCl₃, —OCH₂CHCl₂, —OCH₂CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, R³ is —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In some embodiments, R³ is C1-C6 haloalkyl. In further embodiments, R³ is C1-C4 haloalkyl. In still further embodiments, R³ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R³ is —CF₃, —CHF₂, —CH₂F, —CCl₃, —CHCl₂, or —CH₂Cl.

In some embodiments, R³ is a 3- to 6-membered cycloalkyl. In further embodiments, R³ is a 3- to 5-membered cycloalkyl. In still further embodiments, R³ is a 3- to 4-membered cycloalkyl. In yet further embodiments, R³ is a 3-membered cycloalkyl. In even further embodiments, R³ is a 4-membered cycloalkyl.

In some embodiments, R³ is hydrogen.

In some embodiments, R³ is hydrogen, halogen, (C₁-C₄)alkyl, or 3- to 6-membered cycloalkyl. In further embodiments, R³ is hydrogen.

In further embodiments, R³ is hydrogen, —F, —Cl, methyl, ethyl, n-propyl, isopropyl, or 3- to 6-membered cycloalkyl. In still further embodiments, R³ is hydrogen, —F, —Cl, methyl, ethyl, or 3- to 6-membered cycloalkyl. In yet further embodiments, R³ is hydrogen, —F, —Cl, methyl, or 3- to 6-membered cycloalkyl.

In further embodiments, R³ is hydrogen or (C₁-C₄)alkyl. In still further embodiments, R³ is hydrogen, methyl, ethyl, n-propyl, or isopropyl. In yet further embodiments, R³ is hydrogen, methyl, or ethyl. In even further embodiments, R³ is hydrogen or ethyl. In still further embodiments, R³ is hydrogen or methyl.

In further embodiments, R³ is (C₁-C₄)alkyl. In still further embodiments, R³ is methyl, ethyl, n-propyl, or isopropyl. In yet further embodiments, R³ is methyl or ethyl. In even further embodiments, R³ is ethyl. In still further embodiments, R³ is methyl.

In further embodiments, R³ is (C₁-C₄)alkyl. In still further embodiments, R³ is methyl, ethyl, n-propyl, isopropyl, halogenated methyl, halogenated ethyl, halogenated propyl, CF₃, CCl₃, or CBr₃. In yet further embodiments, R³ is methyl or ethyl. In even further embodiments, R³ is ethyl. In still further embodiments, R³ is methyl. In still further embodiments, R³ is CF₃, CCl₃, or CBr₃.

In further embodiments, R³ is hydrogen or halogen. In still further embodiments, R³ is hydrogen, —F, —Cl, or —Br. In yet further embodiments, R³ is hydrogen, —F, or —Cl. In even further embodiments, R³ is hydrogen or —F. In still further embodiments, R³ is hydrogen or —Cl.

In further embodiments, R³ is halogen. In still further embodiments, R³ is —F, —Cl, or —Br. In yet further embodiments, R³ is —F or —Cl. In even further embodiments, R³ is —F. In still further embodiments, R³ is —Cl.

In further embodiments, R³ is hydrogen or 3- to 6-membered cycloalkyl. In still further embodiments, R³ is hydrogen, cyclopropyl, cyclobutyl, or cyclopentyl. In yet further embodiments, R³ is hydrogen, cyclopropyl, or cyclobutyl. In even further embodiments, R³ is hydrogen or cyclopropyl. In some embodiments, R³ is not a methyl, ethyl or butyl. In some embodiments, R³ is not an acyclic alkyl chain comprising from about 1 to about 5 substituted or unsubstituted carbons.

In further embodiments, R³ is 3- to 6-membered cycloalkyl. In still further embodiments, R³ is 3- to 5-membered cycloalkyl. In yet further embodiments, R³ is 3- to 4-membered cycloalkyl. In even further embodiments, R³ is cyclohexyl. In still further embodiments, R³ is cyclopentyl. In yet further embodiments, R³ is cyclobutyl. In even further embodiments, R³ is cyclopropyl.

In further embodiments, R³ is a 3- to 6-membered cycloalkyl or a C1-C6 haloalkyl. In still further embodiments, R³ is cyclopropyl, cyclobutyl, cyclopentyl, CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R³ is cyclopropyl, cyclobutyl, CF₃, —CHF₂, —CH₂F, —CH₂CF₃, —CH₂CHF₂, —CH₂CH₂F, —CCl₃, —CHCl₂, —CH₂Cl, or —CH₂CCl₃. In even further embodiments, R³ is cyclopropyl, CF₃, —CHF₂, —CH₂F, —CCl₃, or —CHCl₂.

In further embodiments, R³ is a 3-membered cycloalkyl or —CF₃. In still further embodiments, R³ is a 3-membered cycloalkyl. In yet further embodiments, R³ is —CF₃.

h. R^{4a}, R^{4b}, R^{4c}, and R^{4d} Groups (R⁴ Groups)

In some embodiments, each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, each of R^{4a}, R^{4b}, R^{4c}, and R^{4d} is independently selected from hydrogen, F, —Cl, —CN, —NH₂, —OH, —NO₂, methyl, ethyl, n-propyl, isopropyl, ethenyl, propenyl, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, —CH₂CH₂CH₂CN, —CH₂OH, —CH₂CH₂OH, —CH(CH₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, —OCH₂CH₂CH₂Cl, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, each of R^{4a}, R^{4b}, R^{4c}, and R^{4d} is independently selected from hydrogen, F, —Cl, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, —OCH₂CH₂Cl, —NHCH₃, —NHCH₂CH₃, —N(CH₃)₂, and —N(CH₃)CH₂CH₃. In yet further embodiments, each of R^{4a}, R^{4b}, R^{4c}, and R^{4d} is independently selected from hydrogen, F, —Cl, —CN, —NH₂, —OH, —NO₂, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In further embodiments, each of R^{4a}, R^{4b}, R^{4c}, and R^{4d} is hydrogen.

In various embodiments, each of R^{4a}, R^{4b}, R^{4c}, and R^{4d} is independently selected from hydrogen, halogen, —CN, —NH₂, —OH, —NO₂, C1-C4 hydroxyalkyl, C1-C4 haloalkoxy, C1-C4 alkoxy. In further embodiments, each of R^{4a}, R^{4b}, R^{4c}, and R^{4d} is independently selected from hydrogen, F, —Cl, —CN, —NH₂, —OH, —NO₂, —CH₂OH, —CH₂CH₂OH, —CH(CH₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, and —OCH₂CH₂CH₂Cl. In still further embodiments, each of R^{4a}, R^{4b}, R^{4c}, and R^{4d} is independently selected from hydrogen, F, —Cl, —CN, —NH₂, —OH, —NO₂, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, and —OCH₂CH₂Cl. In yet further embodiments, each of R^{4a}, R^{4b}, R^{4c}, and R^{4d} is independently selected from hydrogen, F, —Cl, —CN, —NH₂, —OH, —NO₂, —CH₂OH, methoxy, —OCF₃, —OCHF₂, —OCH₂F, —OCCl₃, —OCHCl₂, and —OCH₂Cl.

In various embodiments, each of R^{4a}, R^{4b}, R^{4c}, and R^{4d} is independently selected from hydrogen, halogen, —CN, —NH₂, —OH, —NO₂, C1-C4 alkylamino, and (C1-C4)(C1-C4) dialkylamino. In further embodiments, each of R^{4a}, R^{4b}, R^{4c}, and R^{4d} is independently selected from hydrogen, F, —Cl, —CN, —NH₂, —OH, —NO₂, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, each of R^{4a}, R^{4b}, R^{4c}, and R^{4d} is independently selected from hydrogen, F, —Cl, —CN, —NH₂, —OH, —NO₂, —NHCH₃, —NHCH₂CH₃, —N(CH₃)₂, and —N(CH₃)CH₂CH₃. In yet further embodiments, each of R^{4a}, R^{4b}, R^{4c}, and R^{4d} is independently selected from hydrogen, F, —Cl, —CN, —NH₂, —OH, —NO₂, —NHCH₃, and —N(CH₃)₂.

In various embodiments, each of R^{4a}, R^{4b}, R^{4c}, and R^{4d} is independently selected from hydrogen, halogen, —CN, —NH₂, —OH, —NO₂, C1-C4 haloalkyl, and C1-C4 cyanoalkyl. In further embodiments, each of R^{4a}, R^{4b}, R^{4c}, and R^{4d} is independently selected from hydrogen, F, —Cl, —CN, —NH₂, —OH, —NO₂, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, and —CH₂CH₂CH₂CN. In still further embodiments, each of R^{4a}, R^{4b}, R^{4c}, and R^{4d} is independently selected from hydrogen, F, —Cl, —CN, —NH₂, —OH, —NO₂, —CH₂F, —CH₂CH₂F, —CH₂Cl, —CH₂CH₂Cl, —CH₂CN, and —CH₂CH₂CN. In yet further embodiments, each of R^{4a}, R^{4b}, R^{4c}, and R^{4d} is independently selected from hydrogen, F, —Cl, —CN, —NH₂, —OH, —NO₂, —CH₂F, —CH₂Cl, and —CH₂CN.

In various embodiments, each of R^{4a}, R^{4b}, R^{4c}, and R^{4d} is independently selected from hydrogen, halogen, —CN, —NH₂, —OH, —NO₂, C1-C4 alkyl, and C2-C4 alkenyl. In further embodiments, each of R^{4a}, R^{4b}, R^{4c}, and R^{4d} is independently selected from hydrogen, F, —Cl, —CN, —NH₂, —OH, —NO₂, methyl, ethyl, n-propyl, isopropyl, ethenyl, and propenyl. In still further embodiments, each of R^{4a}, R^{4b}, R^{4c}, and R^{4d} is independently selected from hydrogen, F, —Cl, —CN, —NH₂, —OH, —NO₂, methyl, ethyl, and ethenyl. In yet further embodiments, each of R^{4a}, R^{4b}, R^{4c}, and R^{4d} is independently selected from hydrogen, F, —Cl, —CN, —NH₂, —OH, —NO₂, and methyl.

In various embodiments, each of R^{4a}, R^{4b}, R^{4c}, and R^{4d} is independently selected from hydrogen and C1-C4 alkyl. In further embodiments, each of R^{4a}, R^{4b}, R^{4c}, and R^{4d} is independently selected from hydrogen, methyl, ethyl, n-propyl, and isopropyl. In still further embodiments, each of R^{4a}, R^{4b}, R^{4c}, and R^{4d} is independently selected from hydrogen, methyl, and ethyl. In yet further embodiments, each of R^{4a}, R^{4b}, R^{4c}, and R^{4d} is independently selected from hydrogen and methyl.

In various embodiments, each of R^{4a}, R^{4b}, R^{4c}, and R^{4d} is independently selected from hydrogen and halogen. In further embodiments, each of R^{4a}, R^{4b}, R^{4c}, and R^{4d} is independently selected from hydrogen, —F, —Cl, and —Br. In still further embodiments, each of R^{4a}, R^{4b}, R^{4c}, and R^{4d} is independently selected from hydrogen, —F, and —Cl. In yet further embodiments, each of R^{4a}, R^{4b}, R^{4c}, and R^{4d} is independently selected from hydrogen and —Cl. In still further embodiments, each of R^{4a}, R^{4b}, R^{4c}, and R^{4d} is independently selected from hydrogen and —F.

In some embodiments, each of R^{4a}, R^{4b}, R^{4c}, and R^{4d} is independently hydrogen, halogen, or C1-C4 alkyl. In further embodiments, each of R^{4a}, R^{4b}, R^{4c}, and R^{4d} is independently hydrogen, —F, —Cl, —Br, methyl, ethyl, n-propyl, or isopropyl. In still further embodiments, each of R^{4a}, R^{4b}, R^{4c}, and R^{4d} is independently hydrogen, —F, —Cl, methyl, and ethyl. In yet further embodiments, each of R^{4a}, R^{4b}, R^{4c}, and R^{4d} is independently hydrogen, —F, and methyl.

i. R⁵ Groups

In some embodiments, R⁵ is selected from —O(CH₂)ₙCy², —NR¹²(CH₂)ₙCy², and Cy¹. In further embodiments, R⁵ is selected from —OCy², —NR¹²Cy², —OCH₂Cy², —NR¹²CH₂Cy², and Cy². In still further embodiments, R⁵ is selected from —OCy², —NR¹²Cy², and Cy².

In some embodiments, R⁵ is selected from —O(CH₂)ₙCy² and —NR¹²(CH₂)ₙCy². In further embodiments, R⁵ is selected from —OCy², —NR¹²Cy², —OCH₂Cy², and —NR¹²CH₂Cy². In still further embodiments, R⁵ is selected from —OCy² and —NR¹²Cy².

In some embodiments, R⁵ is Cy².

j. R^{10a}, R^{10b}, and R^{10c} Groups (R¹⁰ Groups)

In some embodiments, each of R^{10a}, R^{10b}, and R^{10c}, when present, is independently selected from hydrogen and C1-C4 alkyl. In further embodiments, each of R^{10a}, R^{10b}, and R^{10c}, when present, is independently selected from hydrogen, methyl, ethyl, n-propyl, and isopropyl. In still further embodiments, each of R^{10a}, R^{10b}, and R^{10c}, when present, is independently selected from hydrogen, methyl, and ethyl. In yet further embodiments, each of R^{10a}, R^{10b}, and R^{10c}, when present, is independently selected from hydrogen and methyl.

In some embodiments, each occurrence of R¹⁰, when present, is independently hydrogen or (C₁-C₄)alkyl. In further embodiments, each occurrence of R¹⁰, when present, is hydrogen.

In further embodiments, each occurrence of R¹⁰, when present, is independently hydrogen, methyl, ethyl, n-propyl, or isopropyl. In still further embodiments, each occurrence of R¹⁰, when present, is independently hydrogen, methyl, or ethyl. In yet further embodiments, each occurrence of R¹⁰, when present, is independently hydrogen or ethyl. In even further embodiments, each occurrence of R¹⁰, when present, is independently hydrogen or methyl.

In further embodiments, each occurrence of R¹⁰, when present, is (C₁-C₄)alkyl. In even further embodiments, each occurrence of R¹⁰, when present, is independently methyl, ethyl, n-propyl, or isopropyl. In still further embodiments, each occurrence of R¹⁰, when present, is independently methyl or ethyl. In yet further embodiments, each occurrence of R¹⁰, when present, is ethyl. In even further embodiments, each occurrence of R¹⁰, when present, is methyl.

k. R^{11a} and R^{11b} Groups (R¹¹ Groups)

In some embodiments, each of R^{11a} and R^{11b}, when present, is independently selected from hydrogen, C1-C5 alkyl, and C1-C5 hydroxyalkyl. In further embodiments, each of R^{11a} and R^{11b}, when present, is independently selected from hydrogen, methyl, ethyl, n-propyl, isopropyl, n-butyl, isobutyl, sec-butyl, tert-butyl, —CH₂OH, —CH₂CH₂OH, —CH(CH₃)CH₂OH, —CH₂CH₂CH₂OH, —CH(CH₃)CH₂CH₂OH, —CH₂CH(CH₃)CH₂OH, —CH₂CH₂CH₂CH₂OH, and —C(CH₃)₂CH₂OH. In still further embodiments, each of R^{11a} and R^{11b}, when present, is independently selected from hydrogen, methyl, ethyl, n-propyl, isopropyl, —CH₂OH, —CH₂CH₂OH, —CH(CH₃)CH₂OH, and —CH₂CH₂CH₂OH. In yet further embodiments, each of R^{11a} and R^{11b}, when present, is independently selected from hydrogen, methyl, ethyl, —CH₂OH, and —CH₂CH₂OH. In even further embodiments, each of R^{11a} and R^{11b}, when present, is independently selected from hydrogen, methyl, and —CH₂OH. In still further embodiments, each of R^{11a} and R^{11b}, when present, is hydrogen.

In some embodiments, each of R^{11a} and R^{11b}, when present, is independently selected from hydrogen and C1-C5 alkyl. In further embodiments, each of R^{11a} and R^{11b}, when present, is independently selected from hydrogen, methyl, ethyl, n-propyl, isopropyl, n-butyl, isobutyl, sec-butyl, and tert-butyl. In still further embodiments, each of R^{11a} and R^{11b}, when present, is independently selected from hydrogen, methyl, ethyl, n-propyl, and isopropyl. In yet further embodiments, each of R^{11a} and R^{11b}, when present, is independently selected from hydrogen, methyl, and ethyl. In even further embodiments, each of R^{11a} and R^{11b}, when present, is independently selected from hydrogen and methyl.

In some embodiments, each of R^{11a} and R^{11b}, when present, is independently selected from hydrogen and C1-C5 hydroxyalkyl. In further embodiments, each of R^{11a} and R^{11b}, when present, is independently selected from hydrogen, —CH₂OH, —CH₂CH₂OH, —CH(CH₃)CH₂OH, —CH₂CH₂CH₂OH, —CH(CH₃)CH₂CH₂OH, —CH₂CH(CH₃)CH₂OH, —CH₂CH₂CH₂CH₂OH, and —C(CH₃)₂CH₂OH. In still further embodiments, each of R^{11a} and R^{11b}, when present, is independently selected from hydrogen, —CH₂OH, —CH₂CH₂OH, —CH(CH₃)CH₂OH, and —CH₂CH₂CH₂OH. In yet further embodiments, each of R^{11a} and R^{11b}, when present, is independently selected from hydrogen, —CH₂OH, and —CH₂CH₂OH. In even further embodiments, each of R^{11a} and R^{11b}, when present, is independently selected from hydrogen and —CH₂OH.

In some embodiments, each of R^{11a} and R^{11b} together comprise a 3-membered cycloalkyl.

In some embodiments, R¹¹ is hydrogen or (C1-C5)alkyl. In further embodiments, R¹¹ is hydrogen.

In further embodiments, R¹¹ is hydrogen, methyl, ethyl, n-propyl, isopropyl, n-butyl, isobutyl, sec-butyl, or tert-butyl. In still further embodiments, R¹¹ is hydrogen, methyl, ethyl, n-propyl, or isopropyl. In yet further embodiments, R¹¹ is hydrogen, methyl, or ethyl. In even further embodiments, R¹¹ is hydrogen or ethyl. In still further embodiments, R¹¹ is hydrogen or methyl.

l. R¹² Groups

In some embodiments, R¹², when present, is selected from hydrogen and C1-C4 alkyl. In further embodiments, R¹², when present, is selected from hydrogen, methyl, ethyl, n-propyl, and isopropyl. In still further embodiments, R¹², when present, is selected from hydrogen, methyl, and ethyl. In yet further embodiments, R¹², when present, is selected from hydrogen and ethyl. In even further embodiments, R¹², when present, is selected from hydrogen and methyl.

In some embodiments, R¹², when present, is C1-C4 alkyl. In further embodiments, R¹², when present, is selected from methyl, ethyl, n-propyl, and isopropyl. In still further embodiments, R¹², when present, is selected from methyl and ethyl. In yet further embodiments, R¹², when present, is ethyl. In even further embodiments, R¹², when present, is methyl.

In some embodiments, R¹², when present, is hydrogen.

m. R^{13a} and R^{13b} Groups (R¹³ Groups)

In some embodiments, each of R^{13a} and R^{13b}, when present, is independently selected from hydrogen, halogen, —OH, and C1-C4 alkoxy, or wherein each of R^{13a} and R^{13b}, when present, together comprise ═O.

In some embodiments, each of R^{13a} and R^{13b}, when present, is independently selected from hydrogen, halogen, —OH, and C1-C4 alkoxy. In further embodiments, each of R^{13a} and R^{13b}, when present, is independently selected from hydrogen, —F, —Cl, —Br, —OH, methoxy, ethoxy, n-propoxy, and isopropoxy. In still further embodiments, each of R^{13a} and R^{13b}, when present, is independently selected from hydrogen, —F, —Cl, —OH, methoxy, and ethoxy. In yet further embodiments, each of R^{13a} and R^{13b}, when present, is independently selected from hydrogen, —F, —OH, and methoxy.

In some embodiments, each of R^{13a} and R^{13b}, when present, is independently selected from hydrogen, —OH, and C1-C4 alkoxy. In further embodiments, each of R^{13a} and R^{13b}, when present, is independently selected from hydrogen, —OH, methoxy, ethoxy, n-propoxy, and isopropoxy. In still further embodiments, each of R^{13a} and R^{13b}, when present, is independently selected from hydrogen, —OH, methoxy, and ethoxy. In yet further embodiments, each of R^{13a} and R^{13b}, when present, is independently selected from hydrogen, —OH, and methoxy.

In some embodiments, each of R^{13a} and R^{13b}, when present, is independently selected from hydrogen and C1-C4 alkoxy. In further embodiments, each of R^{13a} and R^{13b}, when present, is independently selected from hydrogen, methoxy, ethoxy, n-propoxy, and isopropoxy. In still further embodiments, each of R^{13a} and R^{13b}, when present, is independently selected from hydrogen, methoxy, and ethoxy. In yet further embodiments, each of R^{13a} and R^{13b}, when present, is independently selected from hydrogen and methoxy.

In some embodiments, each of R^{13a} and R^{13b}, when present, is independently selected from hydrogen and —OH. In further embodiments, each of R^{13a} and R^{13b}, when present, is —OH. In still further embodiments, each of R^{13a} and R^{13b}, when present, is hydrogen.

In some embodiments, each of R^{13a} and R^{13b}, when present, is independently selected from hydrogen and halogen. In further embodiments, each of R^{13a} and R^{13b}, when present, is independently selected from hydrogen, —F, —Cl, and —Br. In still further embodiments, each of R^{13a} and R^{13b}, when present, is independently selected from hydrogen, —F, and —Cl. In yet further embodiments, each of R^{13a} and R^{13b}, when present, is independently selected from hydrogen and —F.

In some embodiments, each of R^{13a} and R^{13b}, when present, together comprise ═O.

n. R⁴ Groups

In some embodiments, R¹⁴, when present, is hydrogen, C1-C4 alkyl, C3-C6 cycloalkyl, or —(C1-C4 alkyl)(C3-C6 cycloalkyl). In further embodiments, R¹⁴, when present, is hydrogen, methyl, ethyl, n-propyl, isopropyl, cyclopropyl, cyclobutyl, cyclopentyl, —CH₂(cyclopropyl), —CH₂CH₂(cyclopropyl), —CH₂CH₂CH₂(cyclopropyl), —CH(CH₃)CH₂(cyclopropyl), —CH₂(cyclobutyl), —CH₂CH₂(cyclobutyl), —CH₂CH₂CH₂(cyclobutyl), —CH(CH₃)CH₂(cyclobutyl), —CH₂(cyclopentyl), —CH₂CH₂(cyclopentyl), —CH₂CH₂CH₂(cyclopentyl), or —CH(CH₃)CH₂(cyclopentyl). In still further embodiments, R¹⁴, when present, is hydrogen, methyl, ethyl, cyclopropyl, cyclobutyl, —CH₂(cyclopropyl), —CH₂CH₂(cyclopropyl), —CH₂(cyclobutyl), —CH₂CH₂(cyclobutyl), —CH₂(cyclopentyl), or —CH₂CH₂(cyclopentyl). In yet further embodiments, R¹⁴, when present, is hydrogen, methyl, cyclopropyl, —CH₂(cyclopropyl), —CH₂(cyclobutyl), or —CH₂(cyclopentyl).

In some embodiments, R¹⁴, when present, is hydrogen or C1-C4 alkyl. In further embodiments, R¹⁴, when present, is hydrogen, methyl, ethyl, n-propyl, or isopropyl. In still further embodiments, R¹⁴, when present, is hydrogen, methyl, or ethyl. In yet further embodiments, R¹⁴, when present, is hydrogen or methyl.

In some embodiments, R¹⁴, when present, is C1-C4 alkyl. In further embodiments, R¹⁴, when present, is methyl, ethyl, n-propyl, or isopropyl. In still further embodiments, R¹⁴, when present, is methyl or ethyl. In yet further embodiments, R¹⁴, when present, is methyl.

In some embodiments, R¹⁴, when present, is C3-C6 cycloalkyl or —(C1-C4 alkyl)(C3-C6 cycloalkyl). In further embodiments, R¹⁴, when present, is cyclopropyl, cyclobutyl, cyclopentyl, —CH₂(cyclopropyl), —CH₂CH₂(cyclopropyl), —CH₂CH₂CH₂(cyclopropyl), —CH(CH₃)CH₂(cyclopropyl), —CH₂(cyclobutyl), —CH₂CH₂(cyclobutyl), —CH₂CH₂CH₂(cyclobutyl), —CH(CH₃)CH₂(cyclobutyl), —CH₂(cyclopentyl), —CH₂CH₂(cyclopentyl), —CH₂CH₂CH₂(cyclopentyl), or —CH(CH₃)CH₂(cyclopentyl). In still further embodiments, R¹⁴, when present, is —CH₂(cyclopropyl), —CH₂CH₂(cyclopropyl), —CH₂(cyclobutyl), —CH₂CH₂(cyclobutyl), —CH₂(cyclopentyl), or —CH₂CH₂(cyclopentyl). In yet further embodiments, R¹⁴, when present, is —CH₂(cyclopropyl), —CH₂(cyclobutyl), or —CH₂(cyclopentyl).

In some embodiments, R¹⁴, when present, is hydrogen.

o. Cy¹ Groups

In some embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In further embodiments, Cy^{I}, when present, is selected from a 3- to 10-membered carbocycle, a 3- to 10-membered heterocycle, a 6- to 10-membered aryl, and a 6- to 10-membered heteroaryl, and is substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In yet further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In even further embodiments, Cy¹, when present, is selected from a 3- to 10-membered carbocycle, a 3- to 10-membered heterocycle, a 6- to 10-membered aryl, and a 6- to 10-membered heteroaryl, and is monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is selected from a 3- to 10-membered carbocycle, a 3- to 10-membered heterocycle, a 6- to 10-membered aryl, and a 6- to 10-membered heteroaryl, and is unsubstituted.

In some embodiments, Cy¹, when present, is selected from a 3- to 10-membered carbocycle and a 3- to 10-membered heterocycle, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In further embodiments, Cy^{I}, when present, is selected from a 3- to 10-membered carbocycle and a 3- to 10-membered heterocycle, and is substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is selected from a 3- to 10-membered carbocycle and a 3- to 10-membered heterocycle, and is substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In yet further embodiments, Cy¹, when present, is selected from a 3- to 10-membered carbocycle and a 3- to 10-membered heterocycle, and is substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In even further embodiments, Cy¹, when present, is selected from a 3- to 10-membered carbocycle and a 3- to 10-membered heterocycle, and is monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is selected from a 3- to 10-membered carbocycle and a 3- to 10-membered heterocycle, and is unsubstituted.

In some embodiments, Cy¹, when present, is a 3- to 10-membered carbocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In further embodiments, Cy¹, when present, is a 3- to 10-membered carbocycle substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is a 3- to 10-membered carbocycle substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In yet further embodiments, Cy¹, when present, is a 3- to 10-membered carbocycle substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In even further embodiments, Cy¹, when present, is a 3- to 10-membered carbocycle monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is unsubstituted 3- to 10-membered carbocycle.

In some embodiments, Cy¹, when present, is a 3- to 10-membered heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In further embodiments, Cy¹, when present, is a 3- to 10-membered heterocycle substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is a 3- to 10-membered heterocycle substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In yet further embodiments, Cy¹, when present, is a 3- to 10-membered heterocycle substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In even further embodiments, Cy¹, when present, is a 3- to 10-membered heterocycle monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is unsubstituted 3- to 10-membered heterocycle.

In some embodiments, Cy¹, when present, is selected from a 6- to 10-membered aryl and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In further embodiments, Cy¹, when present, is selected from a 6- to 10-membered aryl and a 6- to 10-membered heteroaryl, and is substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is selected from a 6- to 10-membered aryl and a 6- to 10-membered heteroaryl, and is substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In yet further embodiments, Cy¹, when present, is selected from a 6- to 10-membered aryl and a 6- to 10-membered heteroaryl, and is substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In even further embodiments, Cy¹, when present, is selected from a 6- to 10-membered aryl and a 6- to 10-membered heteroaryl, and is monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is selected from a 6- to 10-membered aryl and a 6- to 10-membered heteroaryl, and is unsubstituted.

In some embodiments, Cy¹, when present, is a 6- to 10-membered aryl substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In further embodiments, Cy^{I}, when present, is a 6- to 10-membered aryl substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is a 6- to 10-membered aryl substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In yet further embodiments, Cy¹, when present, is a 6- to 10-membered aryl substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In even further embodiments, Cy¹, when present, is a 6- to 10-membered aryl monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is an unsubstituted 6- to 10-membered aryl.

In some embodiments, Cy¹, when present, is a 6- to 10-membered heteroaryl substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In further embodiments, Cy¹, when present, is a 6- to 10-membered heteroaryl substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is a 6- to 10-membered heteroaryl substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In yet further embodiments, Cy¹, when present, is a 6- to 10-membered heteroaryl substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In even further embodiments, Cy^{I}, when present, is a 6- to 10-membered heteroaryl monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy². In still further embodiments, Cy¹, when present, is an unsubstituted 6- to 10-membered heteroaryl.

In some embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, In further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is selected from a 3- to 10-membered carbocycle, a 3- to 10-membered heterocycle, a 6- to 10-membered aryl, and a 6- to 10-membered heteroaryl, and is monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino,

In some embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is selected from a 3- to 10-membered carbocycle, a 3- to 10-membered heterocycle, a 6- to 10-membered aryl, and a 6- to 10-membered heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle, a 3- to 10-membered heterocycle, a 6- to 10-membered aryl, and a 6- to 10-membered heteroaryl, and is unsubstituted.

In various embodiments, Cy¹, when present, is selected from a 3- to 10-membered carbocycle and a 3- to 10-membered heterocycle, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy^{I}, when present, is selected from a 3- to 10-membered carbocycle and a 3- to 10-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle and a 3- to 10-membered heterocycle, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is selected from a 3- to 10-membered carbocycle and a 3- to 10-membered heterocycle, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is selected from a 3- to 10-membered carbocycle and a 3- to 10-membered heterocycle, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle and a 3- to 10-membered heterocycle, and is unsubstituted.

In various embodiments, Cy¹, when present, is a 3- to 10-membered carbocycle substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 3- to 10-membered carbocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 3- to 10-membered carbocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 3- to 10-membered carbocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is a 3- to 10-membered carbocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 3- to 10-membered carbocycle.

In various embodiments, Cy¹, when present, is a 9- to 10-membered carbocycle substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 9- to 10-membered carbocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 9- to 10-membered carbocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 9- to 10-membered carbocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is a 9- to 10-membered carbocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 9- to 10-membered carbocycle.

In various embodiments, Cy¹, when present, is a 3- to 10-membered heterocycle substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 3- to 10-membered heterocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 3- to 10-membered heterocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 3- to 10-membered heterocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy^{I}, when present, is a 3- to 10-membered heterocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 3- to 10-membered heterocycle.

In various embodiments, Cy¹, when present, is a 9- to 10-membered heterocycle substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 9- to 10-membered heterocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 9- to 10-membered heterocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 9- to 10-membered heterocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is a 9- to 10-membered heterocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 9- to 10-membered heterocycle.

In various embodiments, Cy¹, when present, is selected from a 6- to 10-membered aryl and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is selected from a 6- to 10-membered aryl and a 6- to 10-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 6- to 10-membered aryl and a 6- to 10-membered heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is selected from a 6- to 10-membered aryl and a 6- to 10-membered heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is selected from a 6- to 10-membered aryl and a 6- to 10-membered heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 6- to 10-membered aryl and a 6- to 10-membered heteroaryl, and is unsubstituted.

In various embodiments, Cy¹, when present, is a 6- to 10-membered aryl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. Examples of 6- to 10-membered aryls include, but are not limited to, phenyl and naphthyl. In further embodiments, Cy¹, when present, is a 6- to 10-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 6- to 10-membered aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 6- to 10-membered aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is a 6- to 10-membered aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 6- to 10-membered aryl.

In various embodiments, Cy¹, when present, is a 6-membered aryl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 6-membered aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 6-membered aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is a 6-membered aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 6-membered aryl.

In various embodiments, Cy¹, when present, is a 6- to 10-membered heteroaryl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. Examples of 6- to 10-membered heteroaryls include, but are not limited to, indolyl, benzofuranyl, benzothiophenyl, triazolyl, imidazolyl, oxazolyl, thiazolyl, pyrazolyl, isoxazolyl, isothiazolyl, pyridinyl, quinolinyl, and isoquinolinyl. In further embodiments, Cy¹, when present, is a 6- to 10-membered heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 6- to 10-membered heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 6- to 10-membered heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is a 6- to 10-membered heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 6- to 10-membered heteroaryl.

In further embodiments, Cy¹, when present, is a structure represented by a formula selected from:

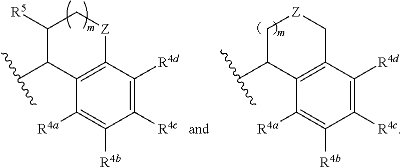

wherein m is 0 or 1; wherein Z is O, CR^{13a}R^{13b}, or NR¹⁴; wherein each of R^{13a} and R^{13b}, when present, is independently selected from hydrogen, halogen, —OH, and C1-C4 alkoxy, or wherein each of R^{13a} and R^{13b}, when present, together comprise ═O; and wherein R¹⁴, when present, is selected from —C(O)(C1-C4 alkyl), C1-C4 alkyl, and C2-C4 alkenyl; wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R⁵, when present, is selected from hydrogen, —O(CH₂)ₙCy², —NR¹²(CH₂)ₙCy², and Cy².

In further embodiments, Cy¹, when present, is a structure represented by a formula selected from:

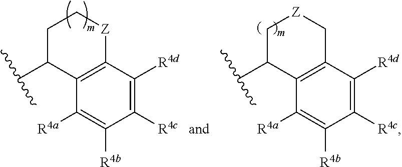

wherein Z is O, CH₂, or NR⁴; wherein R¹⁴, when present, is selected from —C(O)(C1-C4 alkyl), C1-C4 alkyl, and C2-C4 alkenyl; wherein n is 0 or 1; and wherein each of R^{4a}, R^{4b}, R^{4c}, and R^{4d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, Cy¹, when present, is a structure represented by a formula selected from:

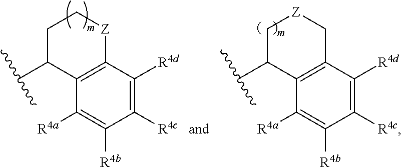

wherein Z is O or CH₂; wherein n is 0 or 1; and wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

Thus, in some embodiments, n is 0 or 1. In further embodiments, n is 0. In still further embodiments, n is 1.

In further embodiments, Cy¹, when present, is a structure represented by a formula:

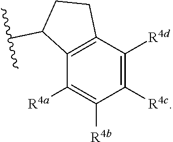

In further embodiments, Cy¹, when present, is a structure represented by a formula selected from:

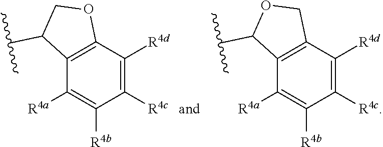

In further embodiments, Cy¹, when present, is a structure represented by a formula:

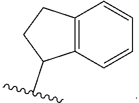 [SMILES: *C1CCc2ccccc21]

In further embodiments, Cy¹, when present, is a structure represented by a formula selected from:

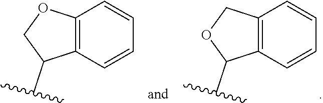 [SMILES: *C1COc2ccccc21 ; *C1OCc2ccccc21]

In further embodiments, Cy¹, when present, is a structure represented by a formula selected from:

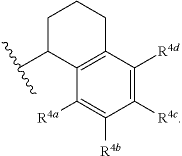

In further embodiments, Cy¹, when present, is a structure represented by a formula selected from:

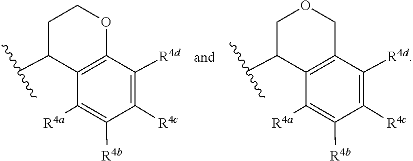

In further embodiments, Cy¹, when present, is a structure represented by a formula selected from:

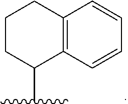 [SMILES: *C1CCCc2ccccc21]

In further embodiments, Cy¹, when present, is a structure represented by a formula selected from:

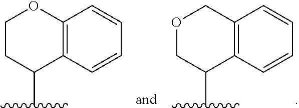 [SMILES: *C1CCOc2ccccc21 ; *C1COCc2ccccc21]

In some embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino. In further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino. In still further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino. In yet further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino. In even further embodiments, Cy¹, when present, is selected from a 3- to 10-membered carbocycle, a 3- to 10-membered heterocycle, a 6- to 10-membered aryl, and a 6- to 10-membered heteroaryl, and is monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino. In still further embodiments, Cy¹, when present, is selected from a 3- to 10-membered carbocycle, a 3- to 10-membered heterocycle, a 6- to 10-membered aryl, and a 6- to 10-membered heteroaryl, and is unsubstituted.

p. Cy² Groups

In some embodiments, Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. Examples of C3-C9 heteroaryls include, but are not limited to, tetrahydrofuran, pyrrolidine, tetrahydrothiophene, piperidine, piperazine, tetrahydropyran, thiane, 1,3-dithiane, 1,4-dithiane, thiomorpholine, dioxane, morpholine, and hexahydro-1H-furo[3,4-c]pyrrole. In further embodiments, Cy² is a C3-C9 heterocycle having at least one O, S, or N atom and substituted with 0, 1, or 2 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy² is a C3-C9 heterocycle having at least one O, S, or N atom and substituted with 0 or 1 group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy² is a C3-C9 heterocycle having at least one O, S, or N atom and monosubstituted with a group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy² is a C3-C9 heterocycle having at least one O, S, or N atom and unsubstituted.

In some embodiments, Cy² is a C3-C9 heterocycle having at least one O atom and substituted with 0, 1, 2, or 3 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy² is a C3-C9 heterocycle having at least one O atom and substituted with 0, 1, or 2 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy² is a C3-C9 heterocycle having at least one O atom and substituted with 0 or 1 group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy² is a C3-C9 heterocycle having at least one O atom and monosubstituted with a group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy² is a C3-C9 heterocycle having at least one O atom and unsubstituted.

In some embodiments, Cy² is a C3-C9 heterocycle having at least one S atom and substituted with 0, 1, 2, or 3 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy² is a C3-C9 heterocycle having at least one S atom and substituted with 0, 1, or 2 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy² is a C3-C9 heterocycle having at least one S atom and substituted with 0 or 1 group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy² is a C3-C9 heterocycle having at least one S atom and monosubstituted with a group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy² is a C3-C9 heterocycle having at least one S atom and unsubstituted.

In some embodiments, Cy² is a C3-C9 heterocycle having at least one N atom and substituted with 0, 1, 2, or 3 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy² is a C3-C9 heterocycle having at least one N atom and substituted with 0, 1, or 2 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy² is a C3-C9 heterocycle having at least one N atom and substituted with 0 or 1 group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy² is a C3-C9 heterocycle having at least one N atom and monosubstituted with a group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy² is a C3-C9 heterocycle having at least one N atom and unsubstituted.

In some embodiments, Cy² is a structure represented by a formula selected from:

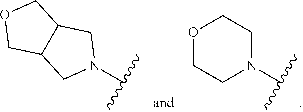 [SMILES: *N1CC2COCC2C1 ; *N1CCOCC1]

In some embodiments, Cy² is a structure represented by a formula:

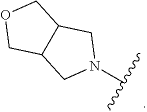 [SMILES: *N1CC2COCC2C1]

In some embodiments, Cy² is a structure represented by a formula:

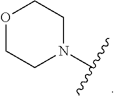 [SMILES: *N1CCOCC1]

### 2. Example Compounds

In some embodiments, a compound can be present as one or more of the following

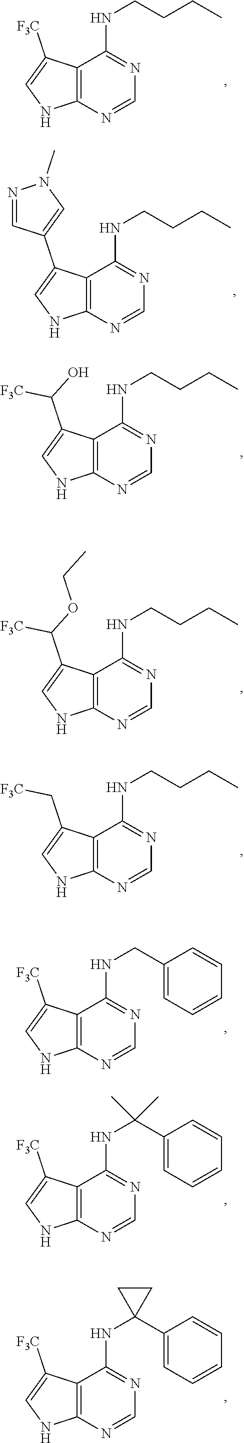

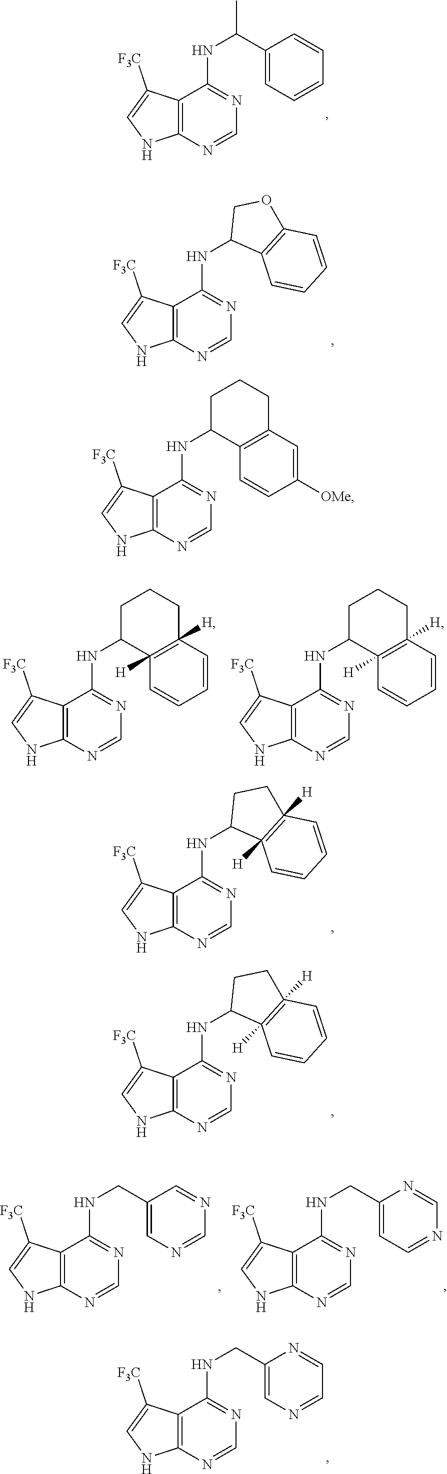

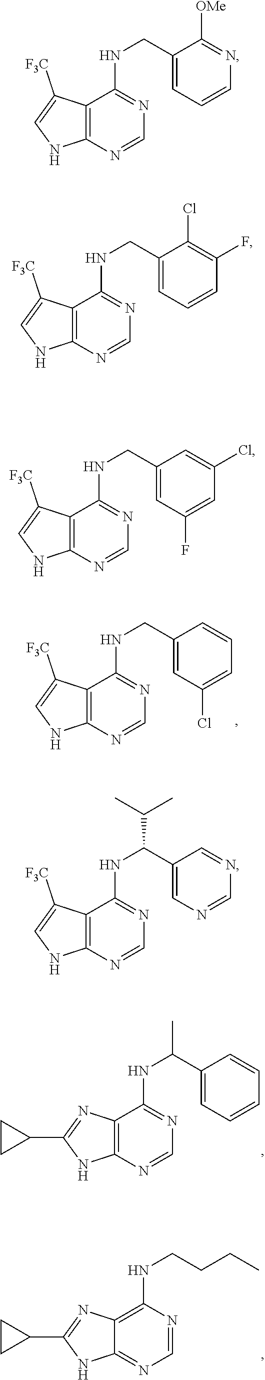

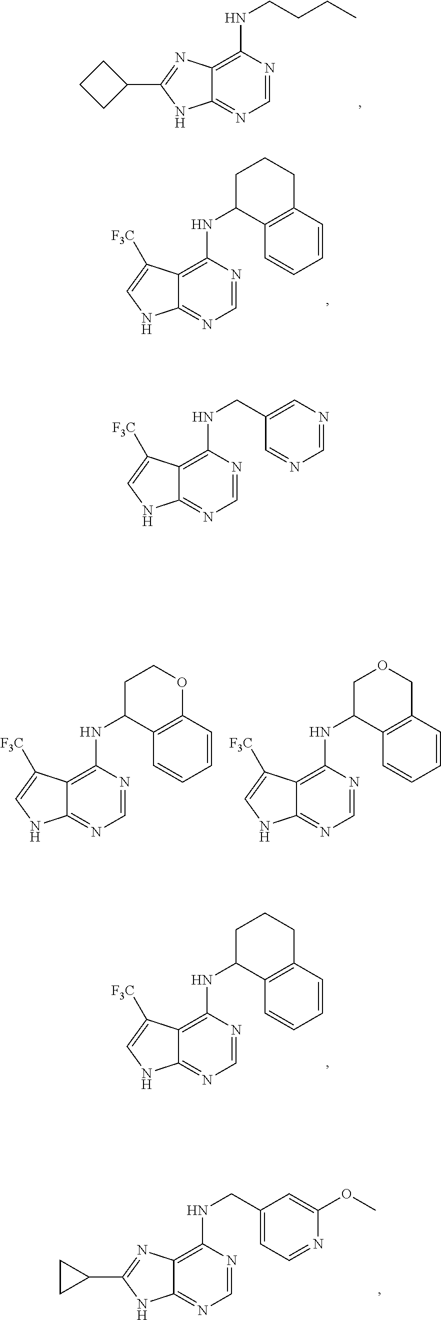

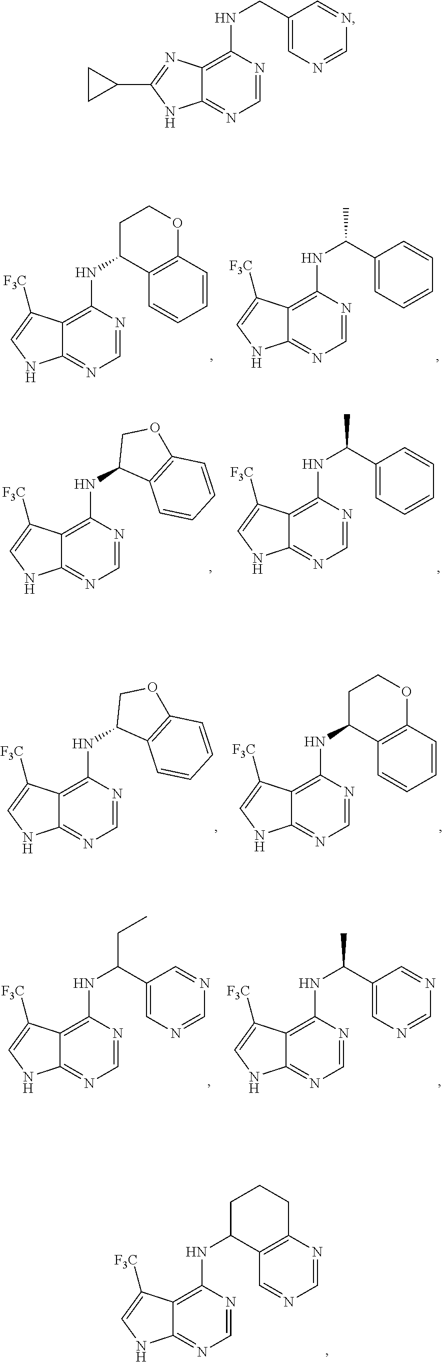

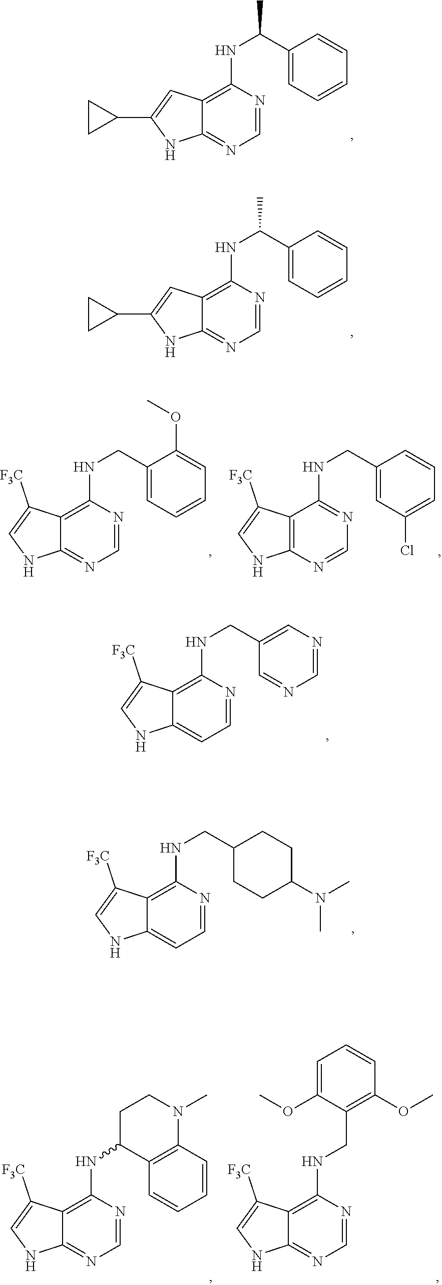

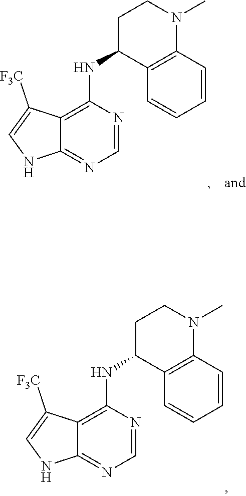 [SMILES: CN1CC[C@H](Nc2ncnc3[nH]cc(C(F)(F)F)c23)c2ccccc21 ; CN1CC[C@@H](Nc2ncnc3[nH]cc(C(F)(F)F)c23)c2ccccc21]

or a pharmaceutically acceptable salt thereof.

In some embodiments, a compound can be present as one or more of the following structures:

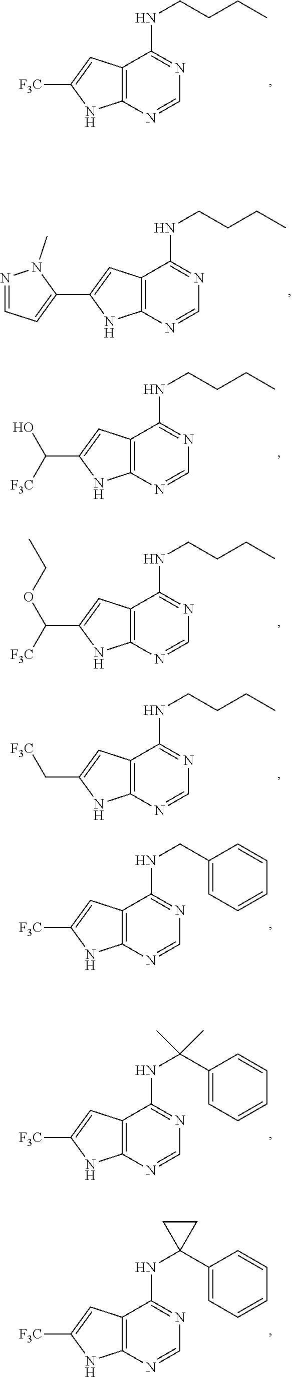

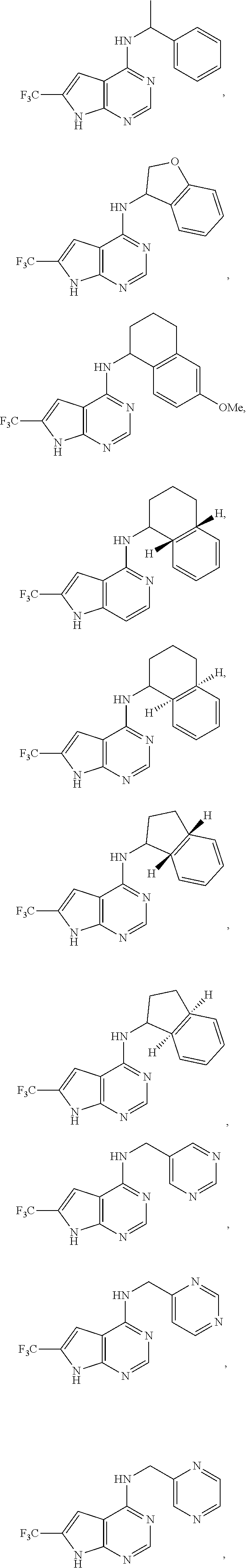

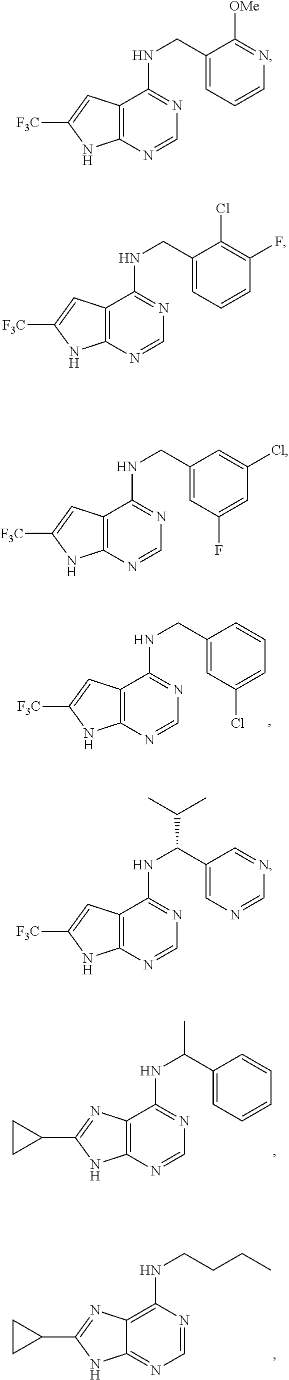

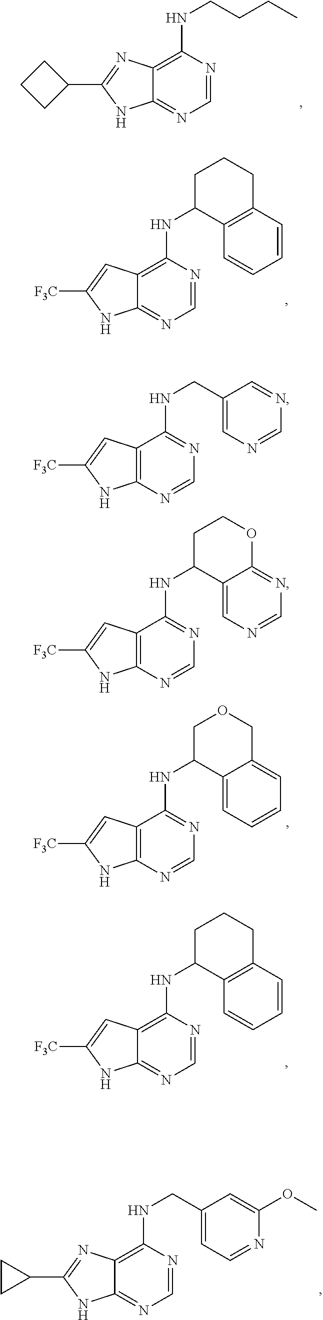

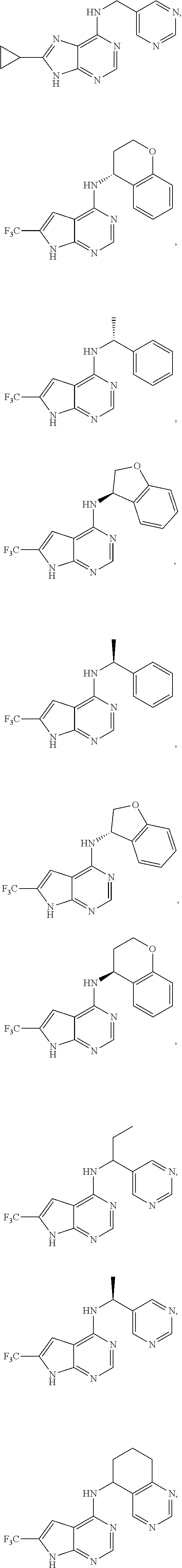

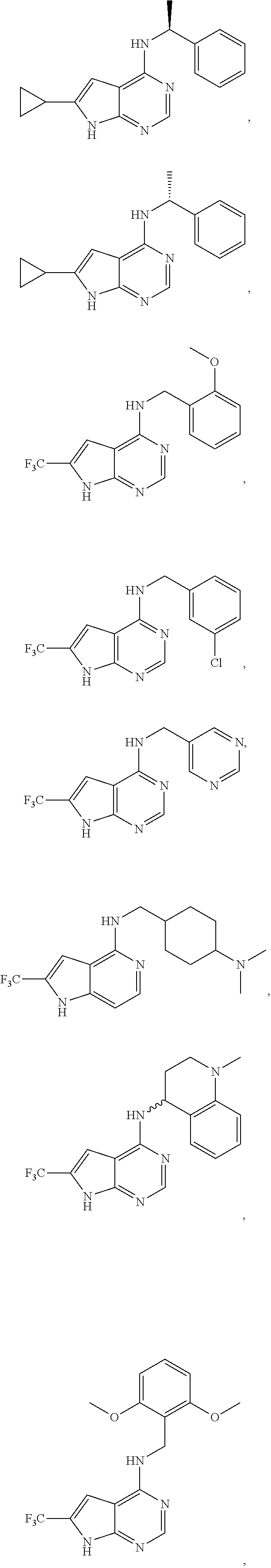

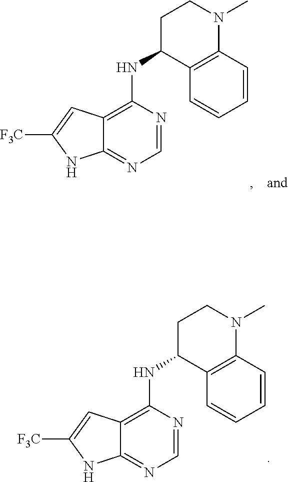 [SMILES: CN1CC[C@H](Nc2ncnc3[nH]c(C(F)(F)F)cc23)c2ccccc21 ; CN1CC[C@@H](Nc2ncnc3[nH]c(C(F)(F)F)cc23)c2ccccc21]

or a pharmaceutically acceptable salt thereof.

In some embodiments, a compound can be present as one or more of the following structures:

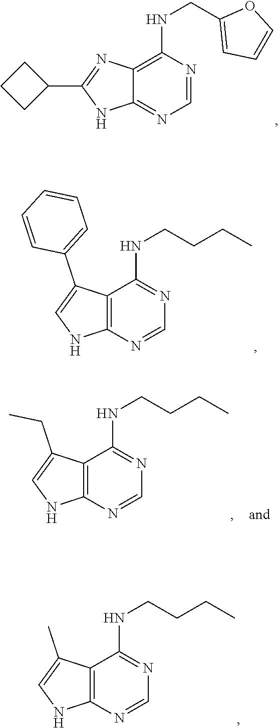

or a pharmaceutically acceptable salt thereof.

In some embodiments, a compound can be present as one or more of the following structures:

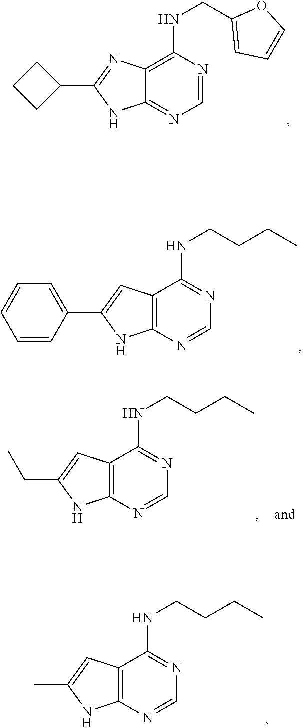

or a pharmaceutically acceptable salt thereof.

In some embodiments, a compound can be present as one or more of the following structures:

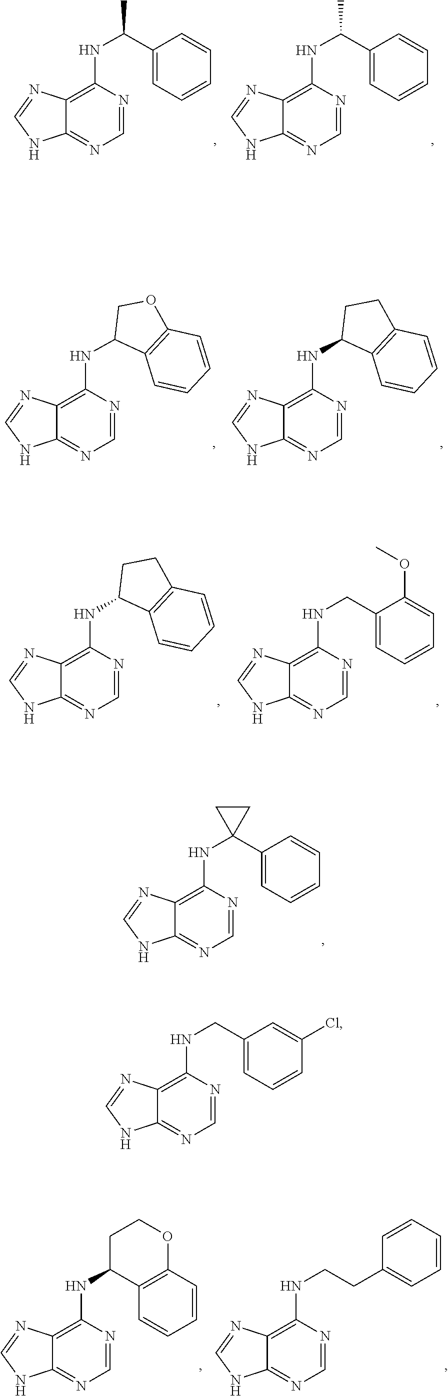

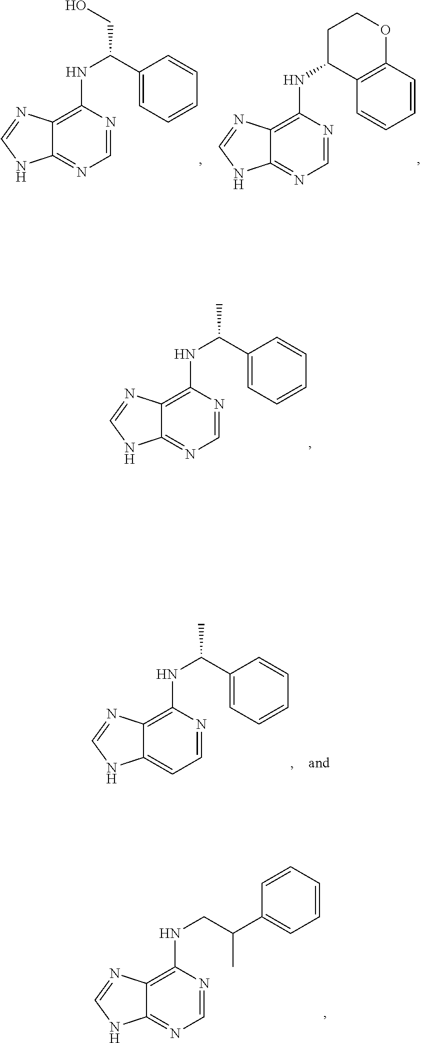

or a pharmaceutically acceptable salt thereof.

In some embodiments, a compound can be present as one or more of the following structures:

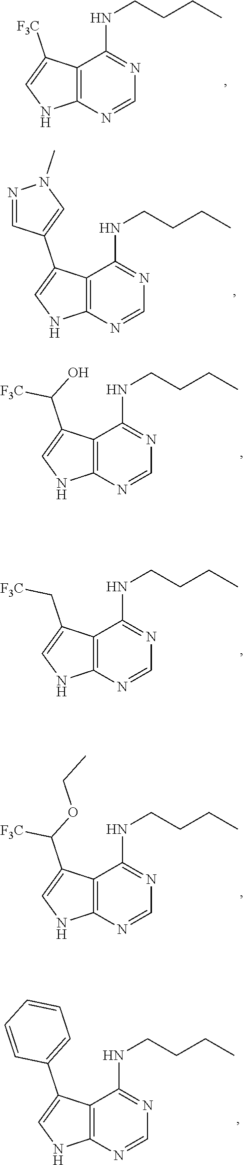

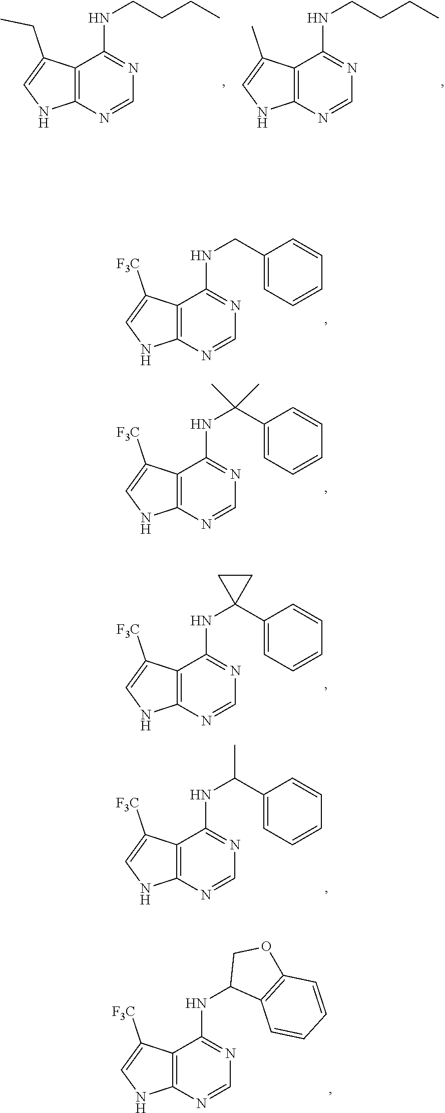

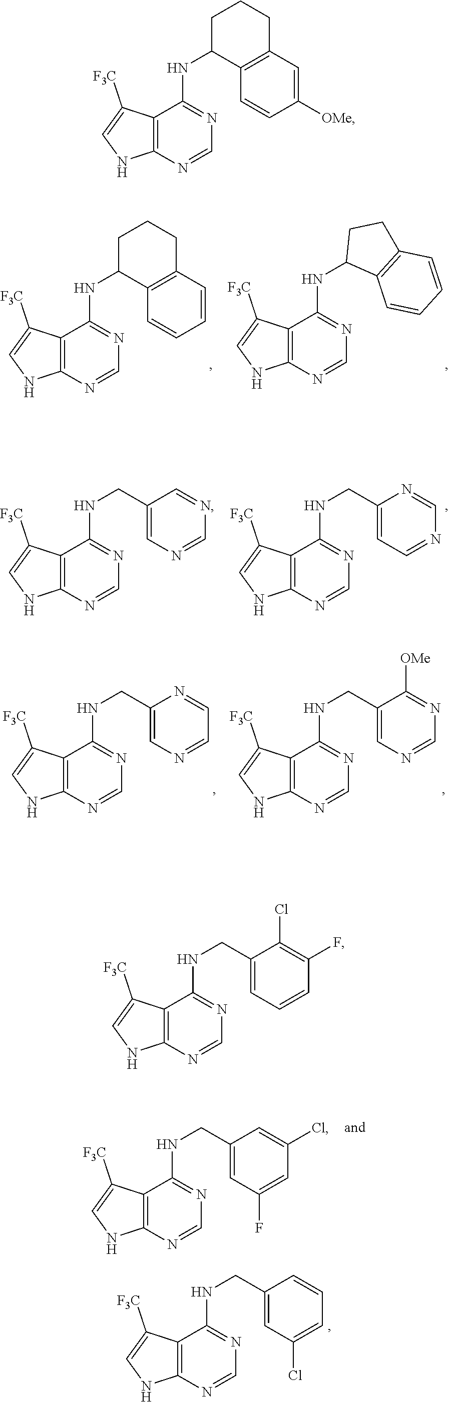

or a pharmaceutically acceptable salt thereof.

In some embodiments, a compound can be present as one or more of the following structures:

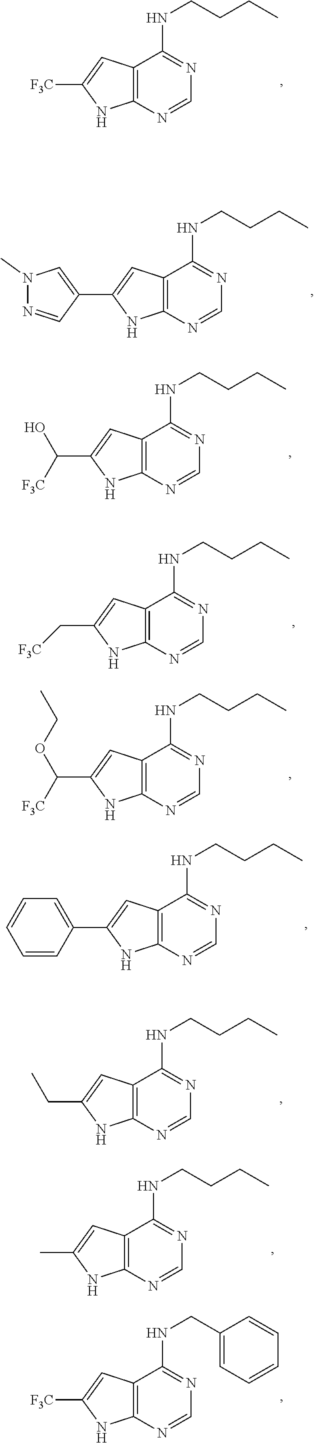

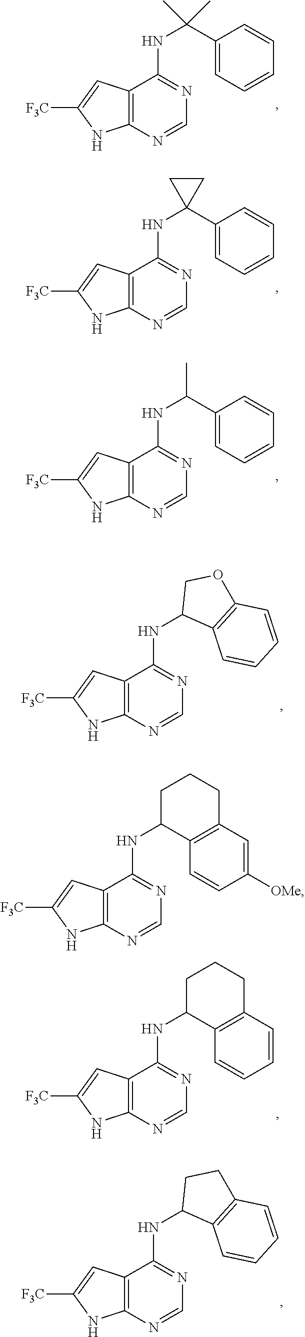

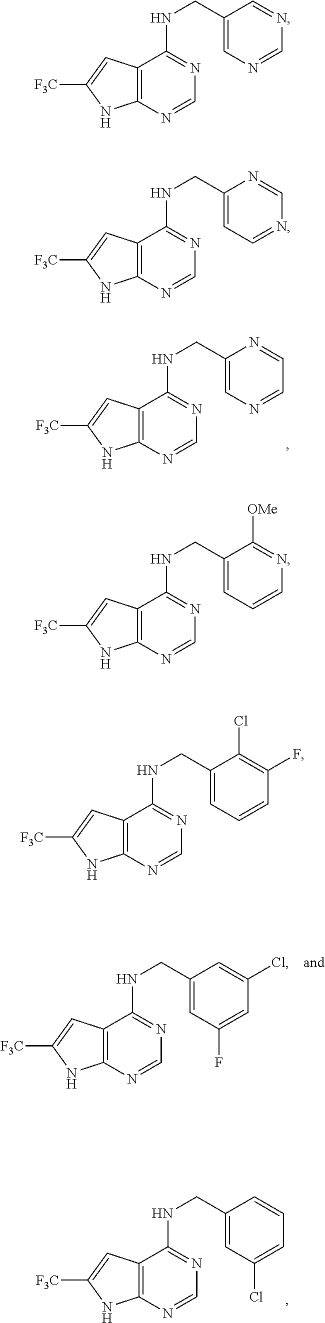

or a pharmaceutically acceptable salt thereof.

In some embodiments, a compound can be present as one or more of the following structures:

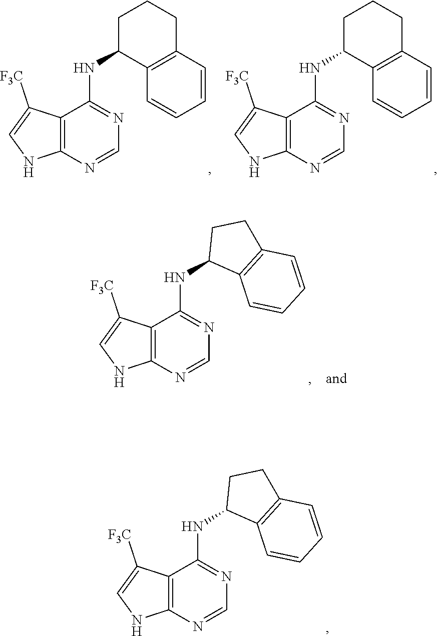

or a pharmaceutically acceptable salt thereof.

In some embodiments, a compound can be present as one or more of the following structures:

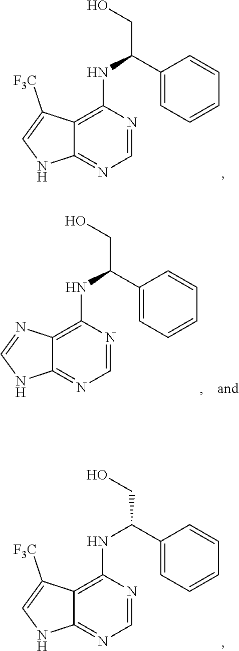 [SMILES: OC[C@H](Nc1ncnc2[nH]cc(C(F)(F)F)c12)c1ccccc1 ; OC[C@H](Nc1ncnc2[nH]cnc12)c1ccccc1 ; OC[C@@H](Nc1ncnc2[nH]cc(C(F)(F)F)c12)c1ccccc1]

or a pharmaceutically acceptable salt thereof.

In some embodiments, a compound can be present as one or more of the following structures:

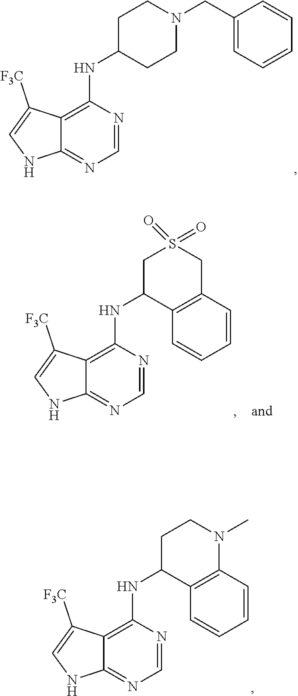 [SMILES: FC(F)(F)c1c[nH]c2ncnc(NC3CCN(Cc4ccccc4)CC3)c12 ; O=S1(=O)Cc2ccccc2C(Nc2ncnc3[nH]cc(C(F)(F)F)c23)C1 ; CN1CCC(Nc2ncnc3[nH]cc(C(F)(F)F)c23)c2ccccc21]

or a pharmaceutically acceptable salt thereof.

In some embodiments, a compound can be present as one or more of the following structures:

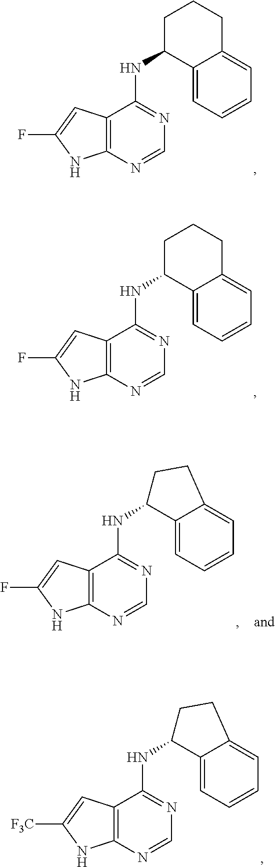 [SMILES: Fc1cc2c(N[C@H]3CCCc4ccccc43)ncnc2[nH]1 ; Fc1cc2c(N[C@@H]3CCCc4ccccc43)ncnc2[nH]1 ; Fc1cc2c(N[C@@H]3CCc4ccccc43)ncnc2[nH]1 ; FC(F)(F)c1cc2c(N[C@@H]3CCc4ccccc43)ncnc2[nH]1]

or a pharmaceutically acceptable salt thereof.

In some embodiments, a compound can be present as one or more of the following structures:

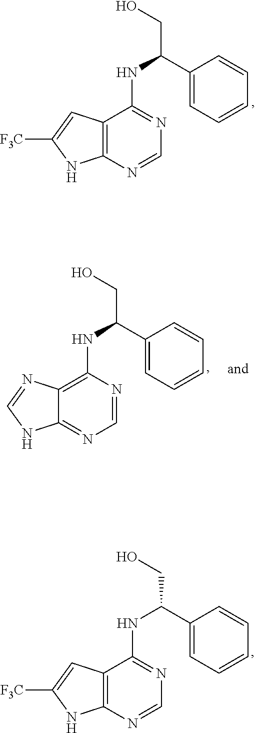 [SMILES: OC[C@H](Nc1ncnc2[nH]c(C(F)(F)F)cc12)c1ccccc1 ; OC[C@H](Nc1ncnc2[nH]cnc12)c1ccccc1 ; OC[C@@H](Nc1ncnc2[nH]c(C(F)(F)F)cc12)c1ccccc1]

or a pharmaceutically acceptable salt thereof.

In some embodiments, a compound can be present as one or more of the following structures:

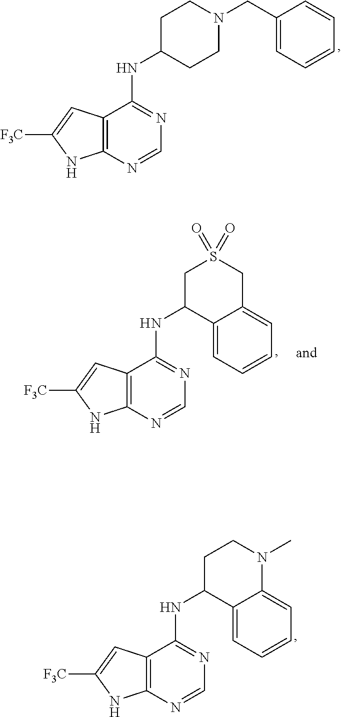 [SMILES: FC(F)(F)c1cc2c(NC3CCN(Cc4ccccc4)CC3)ncnc2[nH]1 ; O=S1(=O)Cc2ccccc2C(Nc2ncnc3[nH]c(C(F)(F)F)cc23)C1 ; CN1CCC(Nc2ncnc3[nH]c(C(F)(F)F)cc23)c2ccccc21]

or a pharmaceutically acceptable salt thereof.

In some embodiments, a compound can be present as one or more of the following structures:

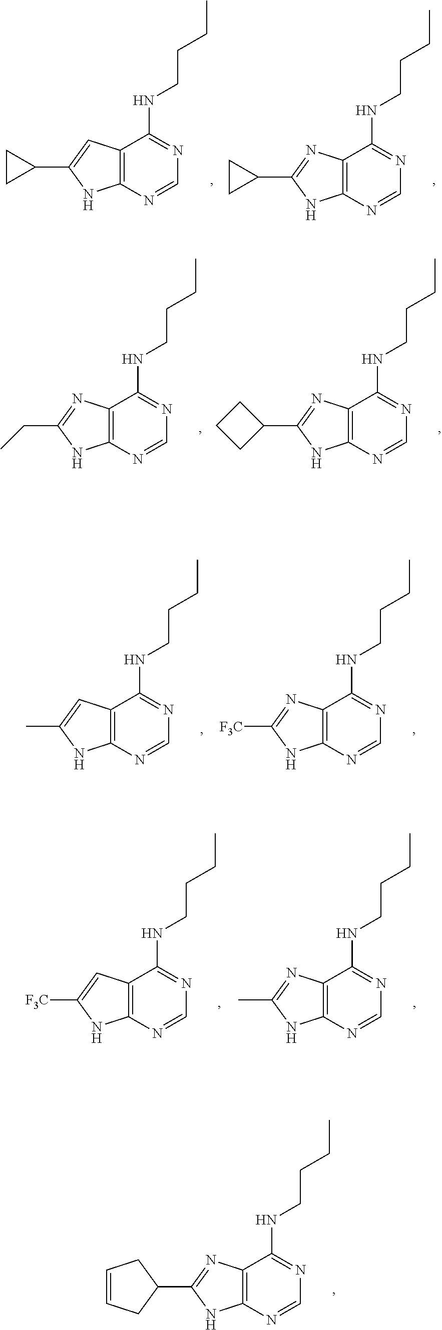

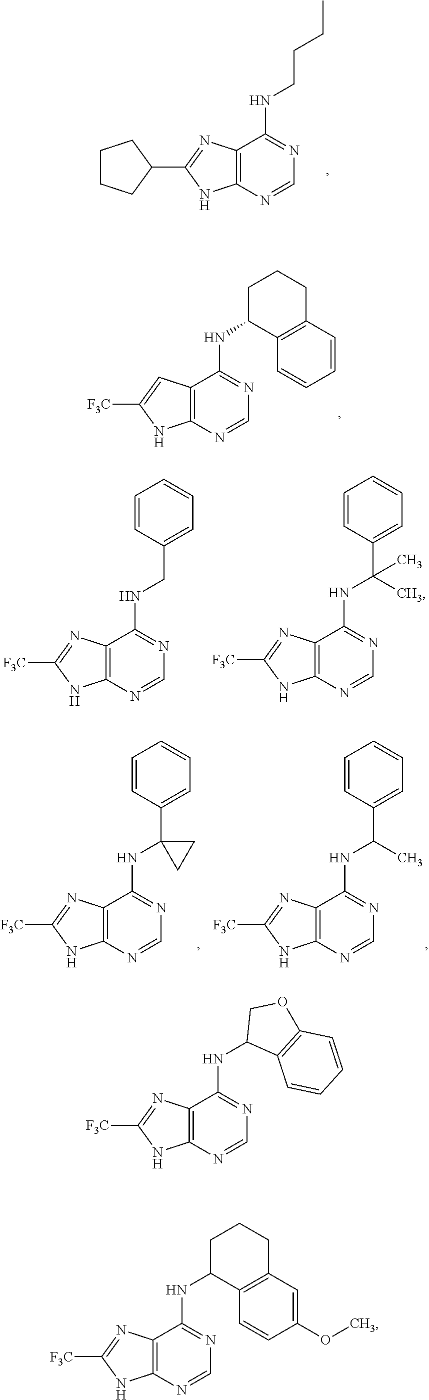

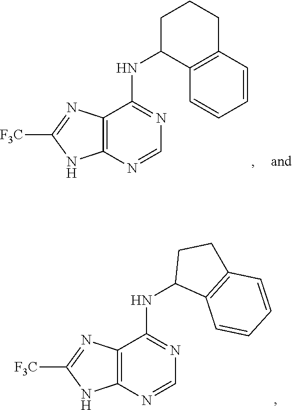 [SMILES: FC(F)(F)c1nc2c(NC3CCCc4ccccc43)ncnc2[nH]1 ; FC(F)(F)c1nc2c(NC3CCc4ccccc43)ncnc2[nH]1]

or a pharmaceutically acceptable salt thereof.

In some embodiments, a compound can be present as:

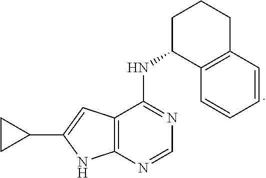 [SMILES: c1ccc2c(c1)CCC[C@H]2Nc1ncnc2[nH]c(C3CC3)cc12]

In some embodiments, a compound can be present as:

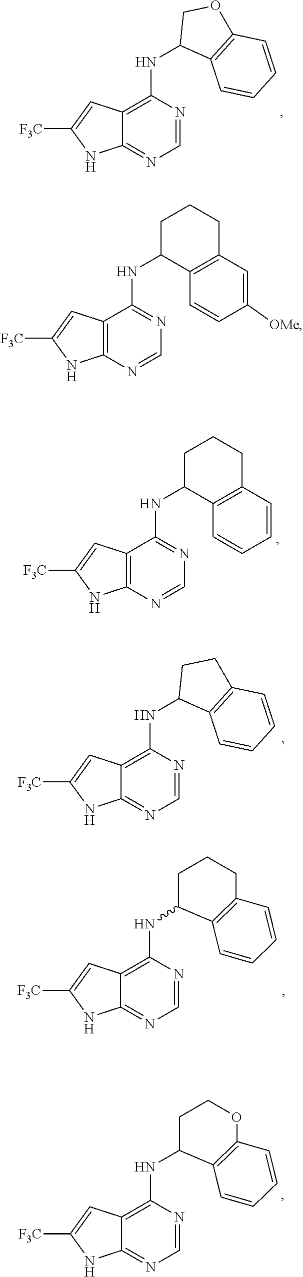 [SMILES: FC(F)(F)c1cc2c(NC3COc4ccccc43)ncnc2[nH]1 ; COc1ccc2c(c1)CCCC2Nc1ncnc2[nH]c(C(F)(F)F)cc12 ; FC(F)(F)c1cc2c(NC3CCCc4ccccc43)ncnc2[nH]1 ; FC(F)(F)c1cc2c(NC3CCc4ccccc43)ncnc2[nH]1 ; FC(F)(F)c1cc2c(NC3CCCc4ccccc43)ncnc2[nH]1 ; FC(F)(F)c1cc2c(NC3CCOc4ccccc43)ncnc2[nH]1]

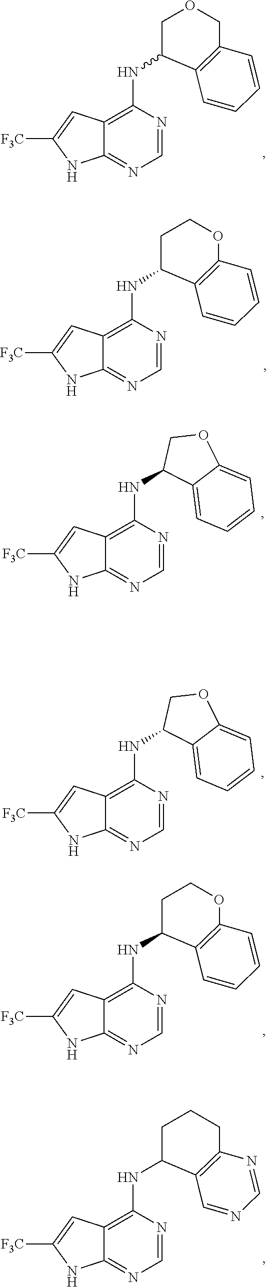 [SMILES: FC(F)(F)c1cc2c(NC3COCc4ccccc43)ncnc2[nH]1 ; FC(F)(F)c1cc2c(N[C@@H]3CCOc4ccccc43)ncnc2[nH]1 ; FC(F)(F)c1cc2c(N[C@H]3COc4ccccc43)ncnc2[nH]1 ; FC(F)(F)c1cc2c(N[C@@H]3COc4ccccc43)ncnc2[nH]1 ; FC(F)(F)c1cc2c(N[C@H]3CCOc4ccccc43)ncnc2[nH]1 ; FC(F)(F)c1cc2c(NC3CCCc4ncncc43)ncnc2[nH]1]

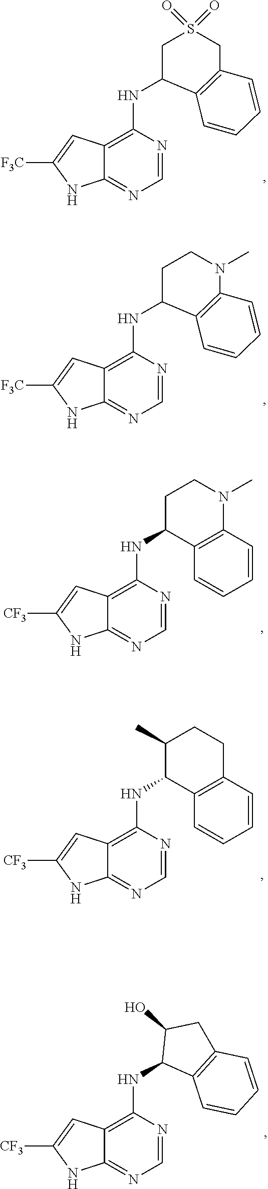 [SMILES: O=S1(=O)Cc2ccccc2C(Nc2ncnc3[nH]c(C(F)(F)F)cc23)C1 ; CN1CCC(Nc2ncnc3[nH]c(C(F)(F)F)cc23)c2ccccc21 ; CN1CC[C@H](Nc2ncnc3[nH]c(C(F)(F)F)cc23)c2ccccc21 ; C[C@H]1CCc2ccccc2[C@@H]1Nc1ncnc2[nH]c(C(F)(F)F)cc12 ; O[C@H]1Cc2ccccc2[C@H]1Nc1ncnc2[nH]c(C(F)(F)F)cc12]

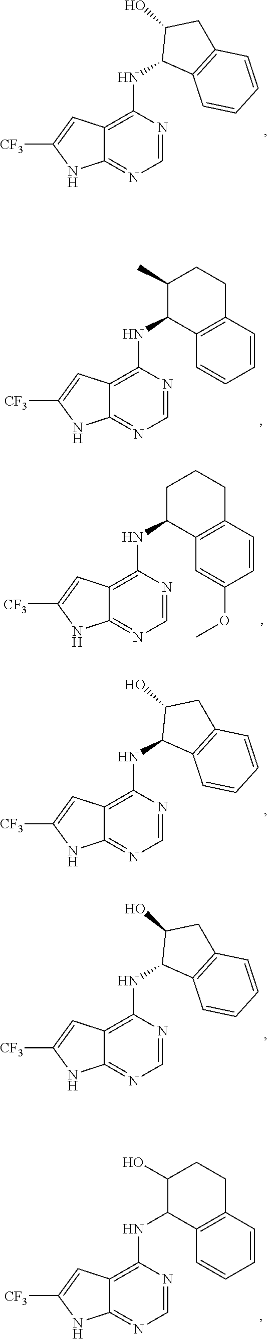 [SMILES: O[C@@H]1Cc2ccccc2[C@@H]1Nc1ncnc2[nH]c(C(F)(F)F)cc12 ; C[C@H]1CCc2ccccc2[C@H]1Nc1ncnc2[nH]c(C(F)(F)F)cc12 ; COc1ccc2c(c1)[C@@H](Nc1ncnc3[nH]c(C(F)(F)F)cc13)CCC2 ; O[C@@H]1Cc2ccccc2[C@H]1Nc1ncnc2[nH]c(C(F)(F)F)cc12 ; O[C@H]1Cc2ccccc2[C@@H]1Nc1ncnc2[nH]c(C(F)(F)F)cc12 ; OC1CCc2ccccc2C1Nc1ncnc2[nH]c(C(F)(F)F)cc12]

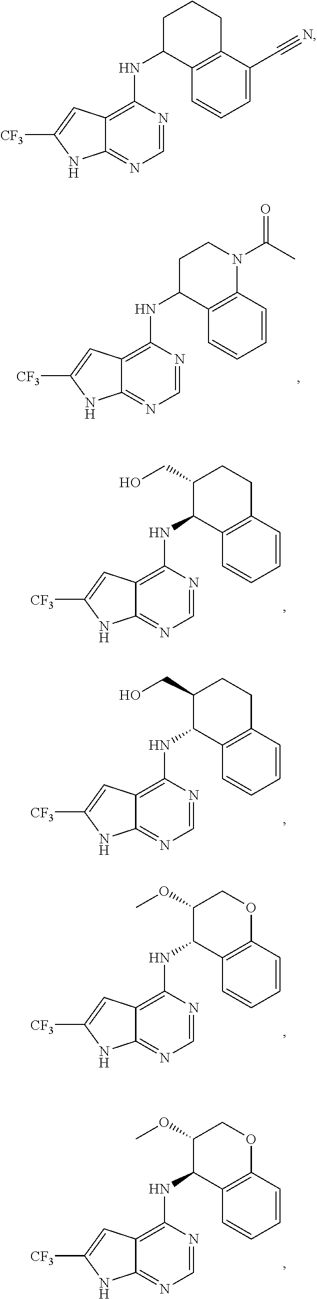

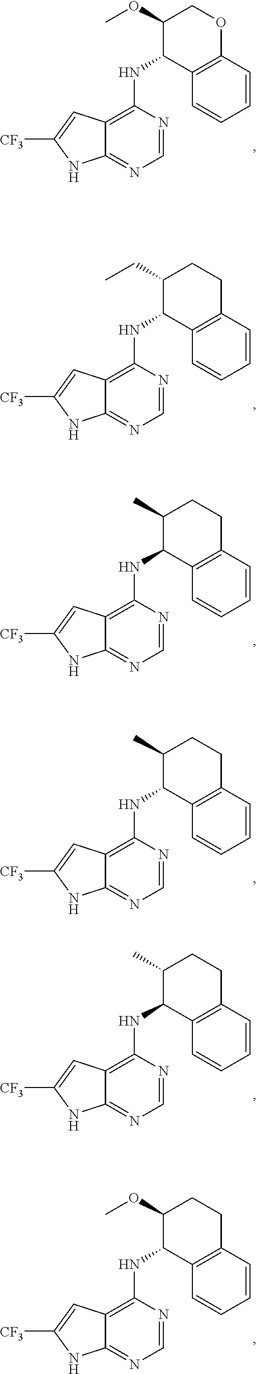 [SMILES: CO[C@H]1COc2ccccc2[C@@H]1Nc1ncnc2[nH]c(C(F)(F)F)cc12 ; CC[C@@H]1CCc2ccccc2[C@@H]1Nc1ncnc2[nH]c(C(F)(F)F)cc12 ; C[C@H]1CCc2ccccc2[C@H]1Nc1ncnc2[nH]c(C(F)(F)F)cc12 ; C[C@H]1CCc2ccccc2[C@@H]1Nc1ncnc2[nH]c(C(F)(F)F)cc12 ; C[C@@H]1CCc2ccccc2[C@H]1Nc1ncnc2[nH]c(C(F)(F)F)cc12 ; CO[C@H]1CCc2ccccc2[C@@H]1Nc1ncnc2[nH]c(C(F)(F)F)cc12]

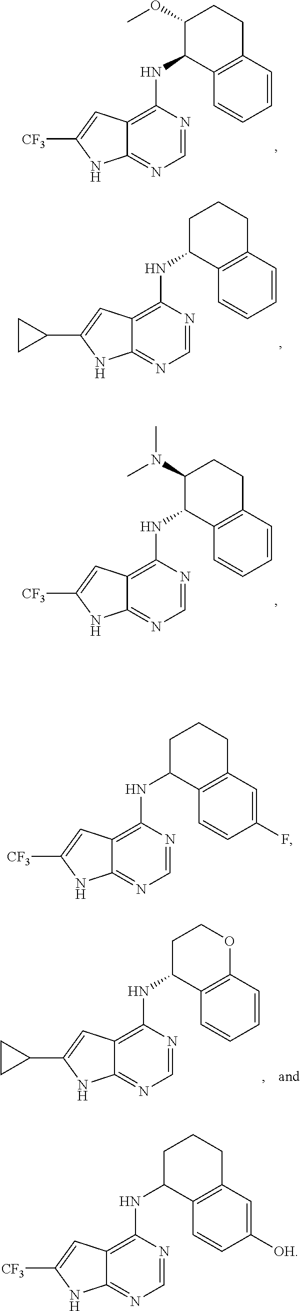 [SMILES: CO[C@@H]1CCc2ccccc2[C@H]1Nc1ncnc2[nH]c(C(F)(F)F)cc12 ; c1ccc2c(c1)CCC[C@H]2Nc1ncnc2[nH]c(C3CC3)cc12 ; CN(C)[C@H]1CCc2ccccc2[C@@H]1Nc1ncnc2[nH]c(C(F)(F)F)cc12 ; Fc1ccc2c(c1)CCCC2Nc1ncnc2[nH]c(C(F)(F)F)cc12 ; c1ccc2c(c1)OCC[C@H]2Nc1ncnc2[nH]c(C3CC3)cc12 ; Oc1ccc2c(c1)CCCC2Nc1ncnc2[nH]c(C(F)(F)F)cc12]

In some embodiments, a compound can be present as one or more of the following

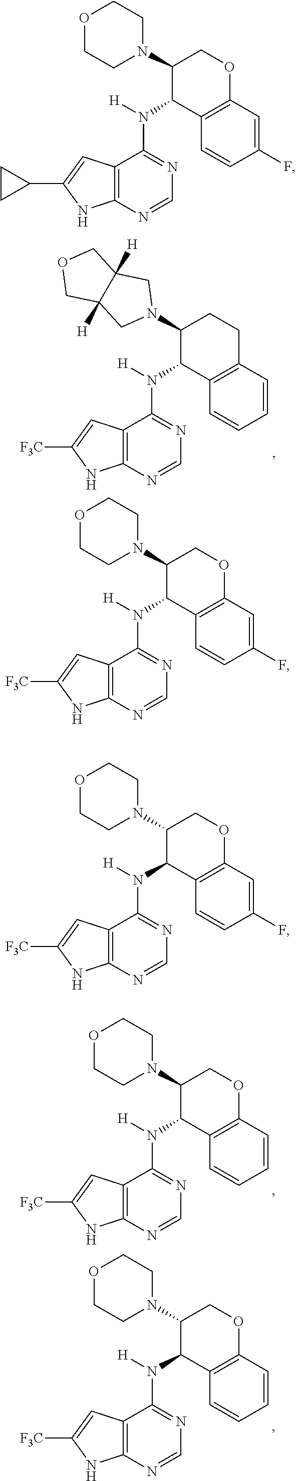

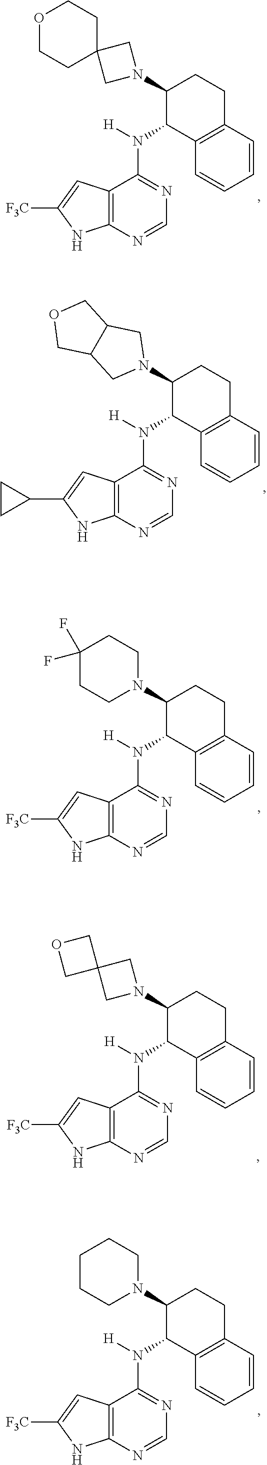 [SMILES: FC(F)(F)c1cc2c(N[C@H]3c4ccccc4CC[C@@H]3N3CC4(CCOCC4)C3)ncnc2[nH]1 ; c1ccc2c(c1)CC[C@H](N1CC3COCC3C1)[C@H]2Nc1ncnc2[nH]c(C3CC3)cc12 ; FC1(F)CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CC4(COC4)C3)ncnc2[nH]1 ; FC(F)(F)c1cc2c(N[C@H]3c4ccccc4CC[C@@H]3N3CCCCC3)ncnc2[nH]1]

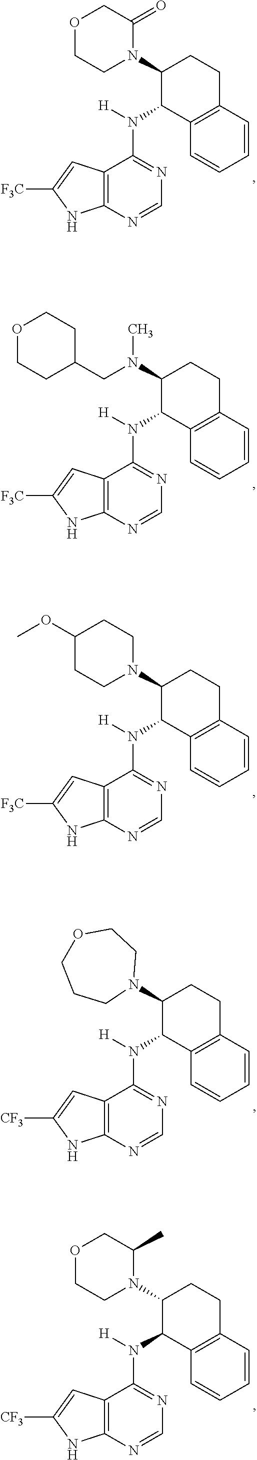 [SMILES: O=C1COCCN1[C@H]1CCc2ccccc2[C@@H]1Nc1ncnc2[nH]c(C(F)(F)F)cc12 ; CN(CC1CCOCC1)[C@H]1CCc2ccccc2[C@@H]1Nc1ncnc2[nH]c(C(F)(F)F)cc12 ; COC1CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CCCOCC3)ncnc2[nH]1 ; C[C@@H]1COCCN1[C@@H]1CCc2ccccc2[C@H]1Nc1ncnc2[nH]c(C(F)(F)F)cc12]

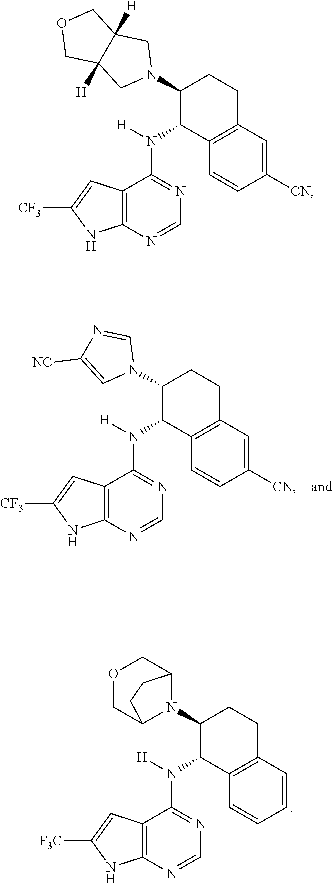 [SMILES: N#Cc1ccc2c(c1)CC[C@H](N1C[C@H]3COC[C@H]3C1)[C@H]2Nc1ncnc2[nH]c(C(F)(F)F)cc12 ; N#Cc1ccc2c(c1)CC[C@@H](n1cnc(C#N)c1)[C@H]2Nc1ncnc2[nH]c(C(F)(F)F)cc12 ; "and" ; FC(F)(F)c1cc2c(N[C@H]3c4ccccc4CC[C@@H]3N3C4CCC3COC4)ncnc2[nH]1]

or a pharmaceutically acceptable salt thereof.

In some embodiments, a compound can be present as one or more of the following

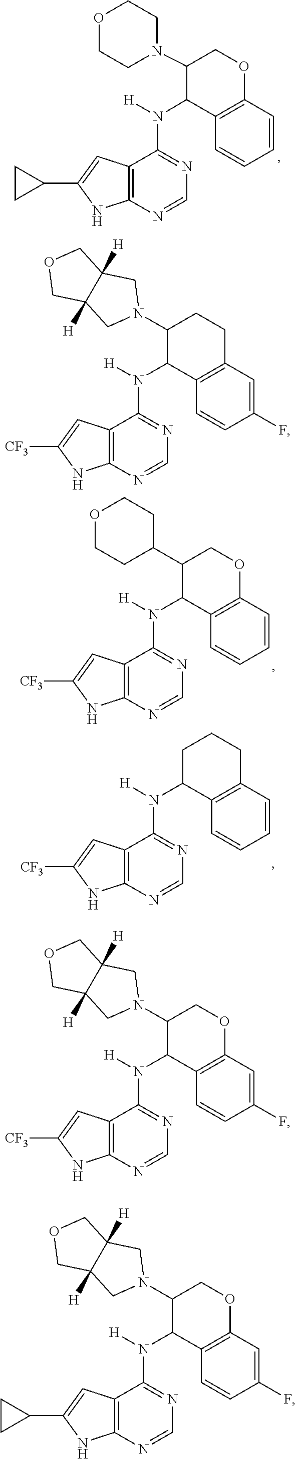

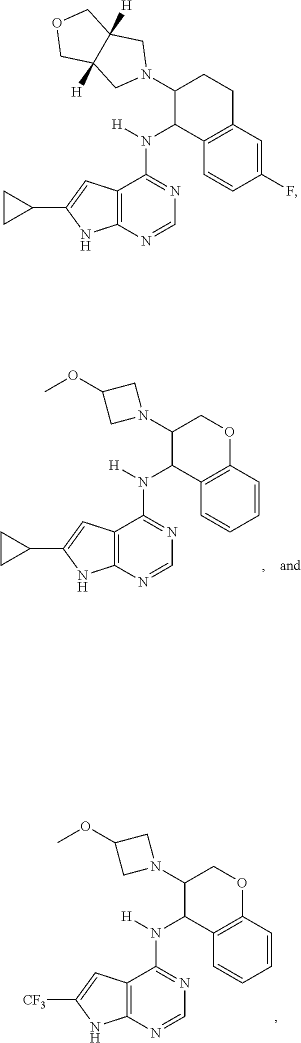 [SMILES: Fc1ccc2c(c1)CCC(N1C[C@H]3COC[C@H]3C1)C2Nc1ncnc2[nH]c(C3CC3)cc12 ; COC1CN(C2COc3ccccc3C2Nc2ncnc3[nH]c(C4CC4)cc23)C1 ; COC1CN(C2COc3ccccc3C2Nc2ncnc3[nH]c(C(F)(F)F)cc23)C1]

or a pharmaceutically acceptable salt thereof.

In some embodiments, a compound can be present as one or more of the following structures:

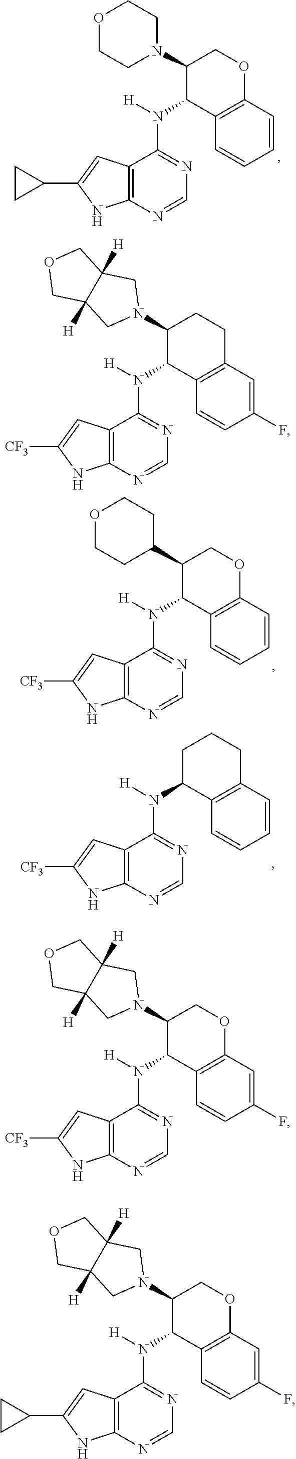

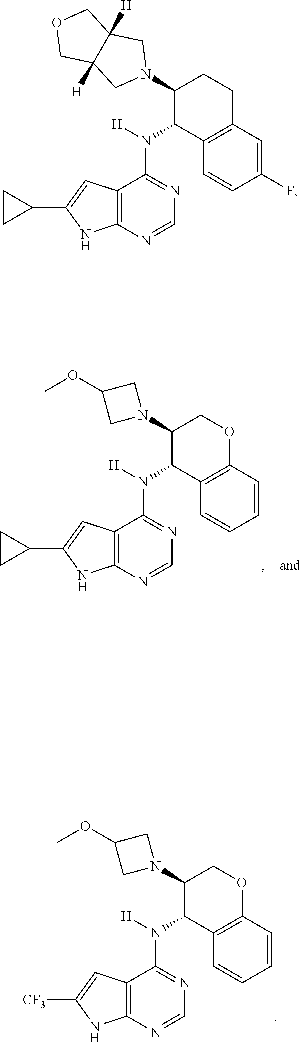 [SMILES: Fc1ccc2c(c1)CC[C@H](N1C[C@H]3COC[C@H]3C1)[C@H]2Nc1ncnc2[nH]c(C3CC3)cc12 ; COC1CN([C@H]2COc3ccccc3[C@@H]2Nc2ncnc3[nH]c(C4CC4)cc23)C1 ; COC1CN([C@H]2COc3ccccc3[C@@H]2Nc2ncnc3[nH]c(C(F)(F)F)cc23)C1]

or a pharmaceutically acceptable salt thereof.

In some embodiments, a compound can be present as one or more of the following

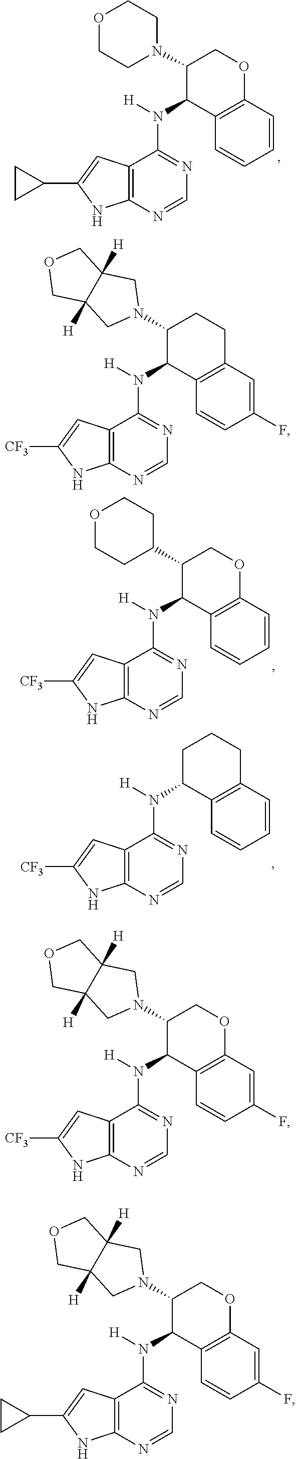

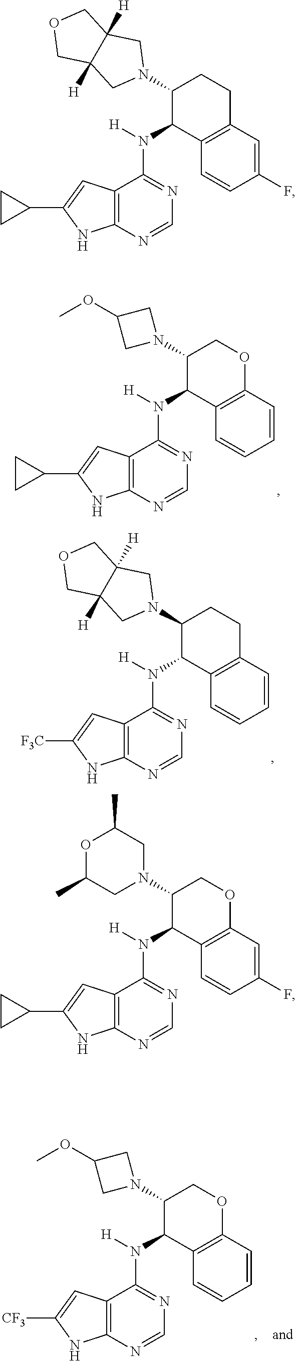

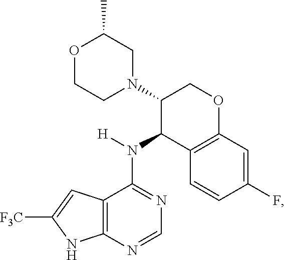 [SMILES: C[C@@H]1CN([C@@H]2COc3cc(F)ccc3[C@H]2Nc2ncnc3[nH]c(C(F)(F)F)cc23)CCO1]

or a pharmaceutically acceptable salt thereof

### 3. Prophetic Compound Examples

The following compound examples are prophetic, and can be prepared using the synthesis methods described herein above and other general methods as needed as would be known to one skilled in the art. It is anticipated that the prophetic compounds would be active as modulators of RNA polymerase-I signaling, and such activity can be determined using the assay methods described herein below.

In one aspect, a compound is selected from:

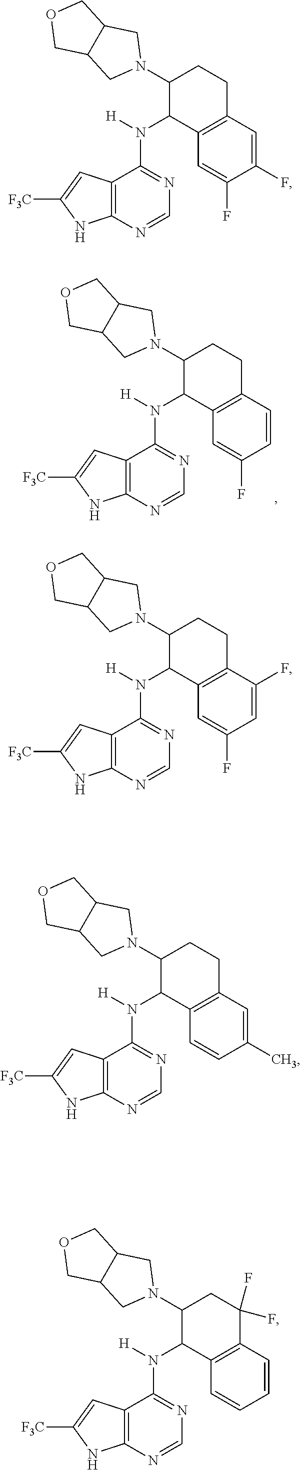 [SMILES: Fc1cc2c(cc1F)C(Nc1ncnc3[nH]c(C(F)(F)F)cc13)C(N1CC3COCC3C1)CC2 ; Fc1ccc2c(c1)C(Nc1ncnc3[nH]c(C(F)(F)F)cc13)C(N1CC3COCC3C1)CC2 ; Fc1cc(F)c2c(c1)C(Nc1ncnc3[nH]c(C(F)(F)F)cc13)C(N1CC3COCC3C1)CC2 ; Cc1ccc2c(c1)CCC(N1CC3COCC3C1)C2Nc1ncnc2[nH]c(C(F)(F)F)cc12 ; FC(F)(F)c1cc2c(NC3c4ccccc4C(F)(F)CC3N3CC4COCC4C3)ncnc2[nH]1]

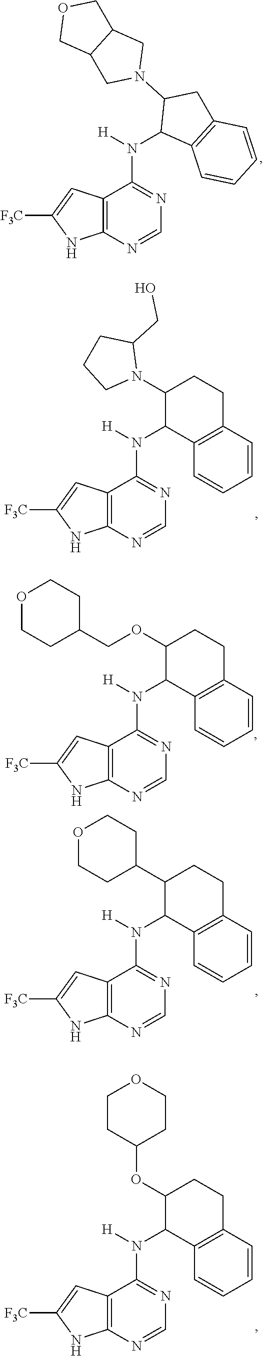

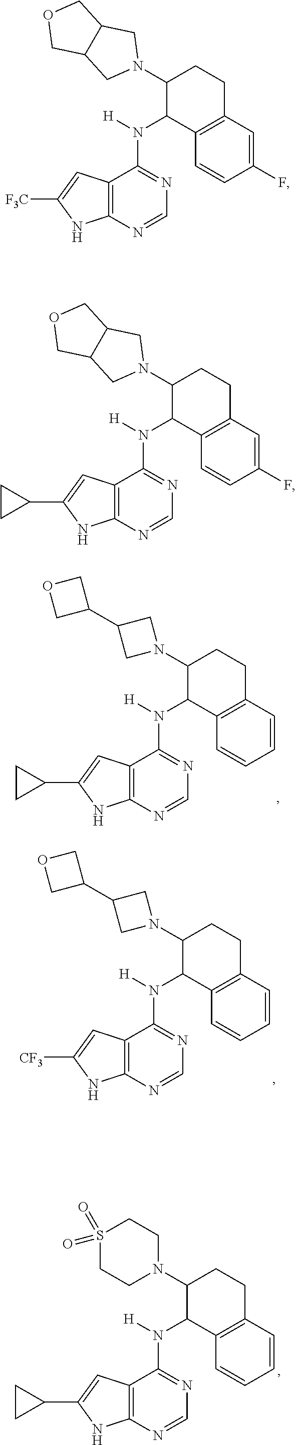 [SMILES: Fc1ccc2c(c1)CCC(N1CC3COCC3C1)C2Nc1ncnc2[nH]c(C(F)(F)F)cc12 ; Fc1ccc2c(c1)CCC(N1CC3COCC3C1)C2Nc1ncnc2[nH]c(C3CC3)cc12 ; c1ccc2c(c1)CCC(N1CC(C3COC3)C1)C2Nc1ncnc2[nH]c(C3CC3)cc12 ; FC(F)(F)c1cc2c(NC3c4ccccc4CCC3N3CC(C4COC4)C3)ncnc2[nH]1 ; O=S1(=O)CCN(C2CCc3ccccc3C2Nc2ncnc3[nH]c(C4CC4)cc23)CC1]

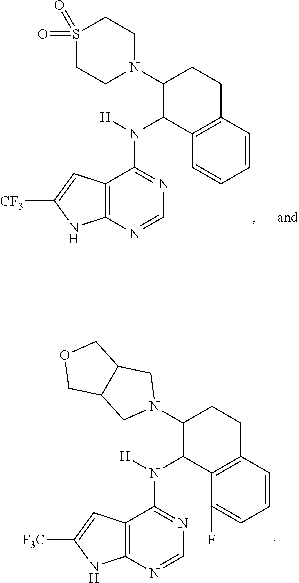 [SMILES: O=S1(=O)CCN(C2CCc3ccccc3C2Nc2ncnc3[nH]c(C(F)(F)F)cc23)CC1 ; Fc1cccc2c1C(Nc1ncnc3[nH]c(C(F)(F)F)cc13)C(N1CC3COCC3C1)CC2]

In one aspect, a compound is selected from:

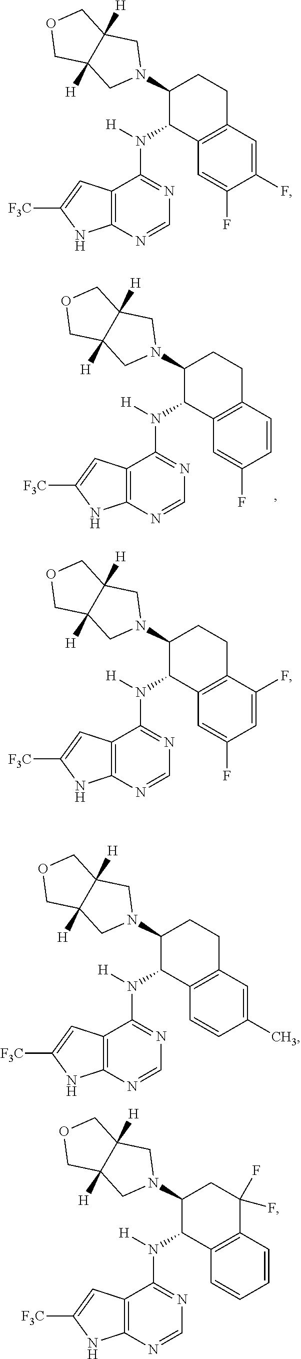

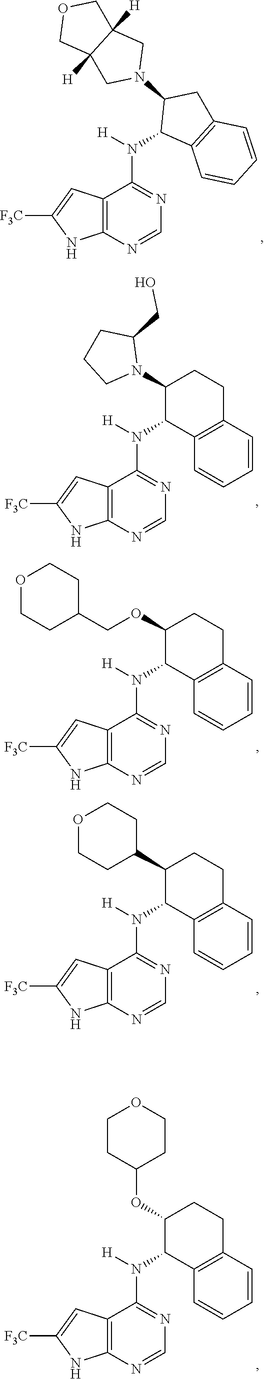

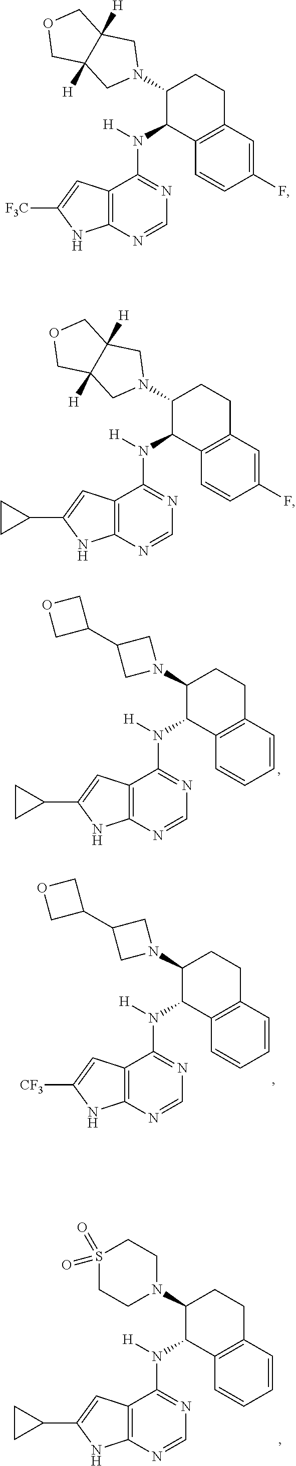 [SMILES: Fc1ccc2c(c1)CC[C@@H](N1C[C@H]3COC[C@H]3C1)[C@@H]2Nc1ncnc2[nH]c(C(F)(F)F)cc12 ; Fc1ccc2c(c1)CC[C@@H](N1C[C@H]3COC[C@H]3C1)[C@@H]2Nc1ncnc2[nH]c(C3CC3)cc12 ; c1ccc2c(c1)CC[C@H](N1CC(C3COC3)C1)[C@H]2Nc1ncnc2[nH]c(C3CC3)cc12 ; FC(F)(F)c1cc2c(N[C@H]3c4ccccc4CC[C@@H]3N3CC(C4COC4)C3)ncnc2[nH]1 ; O=S1(=O)CCN([C@H]2CCc3ccccc3[C@@H]2Nc2ncnc3[nH]c(C4CC4)cc23)CC1]

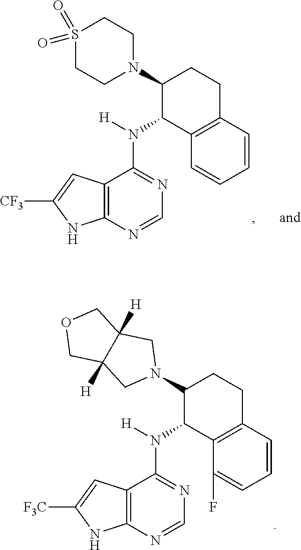

It is contemplated that one or more compounds can optionally be omitted from the disclosed disclosure.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. Methods of Making the Compounds

In various embodiments, the disclosures relates to methods of making compounds useful to treat a disorder associated with PINK1 kinase activity such as, for example, a kidney disease, a fibrotic disorder, or a reperfusion injury. Thus, in some embodiments, disclosed are methods of making a disclosed compound.

Compounds according to the present disclosure can, for example, be prepared by the several methods outlined below. A practitioner skilled in the art will understand the appropriate use of protecting groups [see: Greene and Wuts, Protective Groups in Organic Synthesis] and the preparation of known compounds found in the literature using the standard methods of organic synthesis. There may come from time to time the need to rearrange the order of the recommended synthetic steps, however this will be apparent to the judgment of a chemist skilled in the art of organic synthesis. The following examples are provided so that the disclosure might be more fully understood, are illustrative only, and should not be construed as limiting.

In some embodiments, the disclosed compounds comprise the products of the synthetic methods described herein. In further embodiments, the disclosed compounds comprise a compound produced by a synthetic method described herein. In still further embodiments, the disclosure comprises a pharmaceutical composition comprising a therapeutically effective amount of the product of the disclosed methods and a pharmaceutically acceptable carrier. In still further embodiments, the disclosure comprises a method for manufacturing a medicament comprising combining at least one compound of any of disclosed compounds or at least one product of the disclosed methods with a pharmaceutically acceptable carrier or diluent.

### 1. Route I

In some embodiments, N-containing heteroaryl analogs can be prepared as shown below.

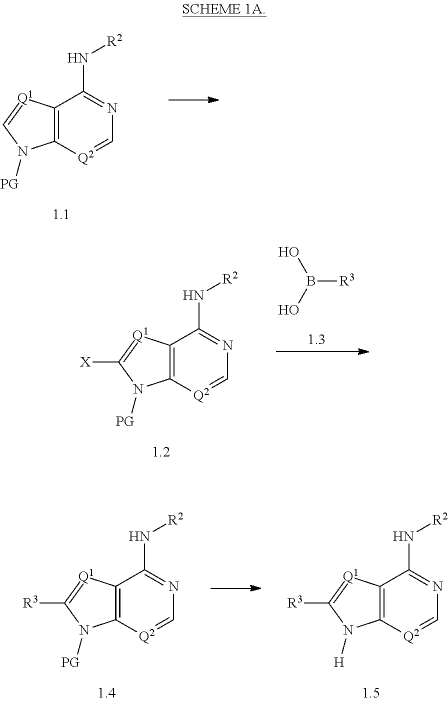

Compounds are represented in generic form, wherein X is a halogen, wherein PG is an amine protecting group, and with substituents as noted in compound descriptions elsewhere herein. A more specific example is set forth below.

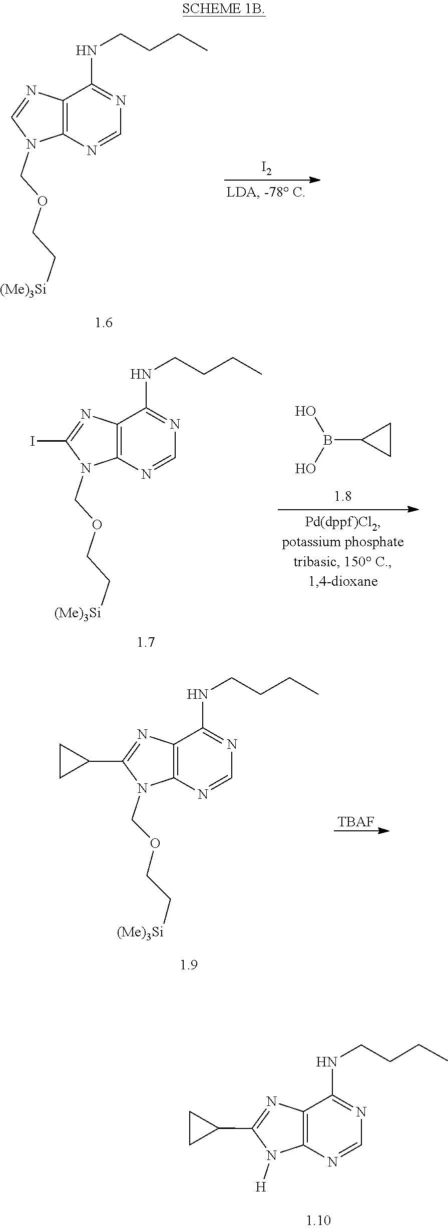

In some embodiments, compounds of type 1.5, and similar compounds, can be prepared according to reaction Scheme 1B above. Thus, compounds of type 1.7 can be prepared by a halogenation reaction of an appropriate N-containing heteroaryl analog, e.g., 1.6 as shown above. Appropriate N-containing heteroaryl analogs are commercially available or prepared by methods known to one skilled in the art. The halogenation reaction is carried out in the presence of an appropriate halide source, e.g., iodine, and an appropriate base, e.g., lithium diisopropylamide (LDA) at an appropriate temperature, e.g., −78° C. Compounds of type 1.9 can be prepared by a coupling reaction of an appropriate halide, e.g., 1.7 as shown above, and an appropriate boronic acid, e.g., 1.8 as shown above. Appropriate boronic acids are commercially available or prepared by methods known to one skilled in the art. The coupling reaction is carried out in the presence of an appropriate catalyst, e.g., [1,1′-Bis(diphenylphosphino)ferrocene]dichloropalladium(II), and an appropriate ligand, e.g., potassium phosphate tribasic, in an appropriate solvent, e.g., 1,4-dioxane, at an appropriate temperature, e.g., 150° C. Compounds of type 1.10 can be prepared by deprotection of an appropriate protected amine, e.g., 1.9 as shown above. The deprotection is carried out in the presence of an appropriate deprotecting agent, e.g., tetrabutylammonium fluoride (TBAF). As can be appreciated by one skilled in the art, the above reaction provides an example of a generalized approach wherein compounds similar in structure to the specific reactants above (compounds similar to compounds of type 1.1, 1.2, 1.3, and 1.4), can be substituted in the reaction to provide substituted N-containing heteroaryl analogs similar to Formula 1.5.

### 2. Route II

In some embodiments, N-containing heteroaryl analogs can be prepared as shown below.

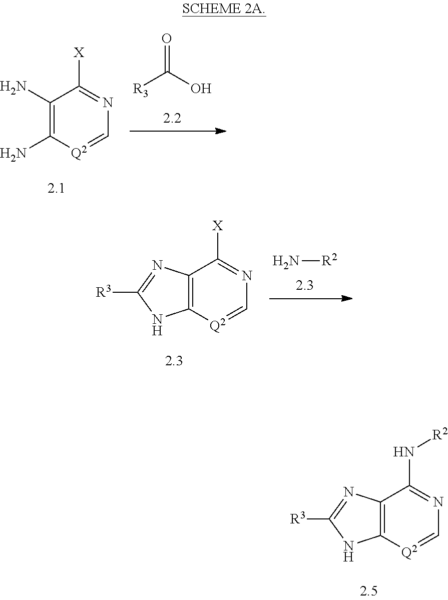

Compounds are represented in generic form, wherein X is a halogen and with substituents as noted in compound descriptions elsewhere herein. A more specific example is set forth below.

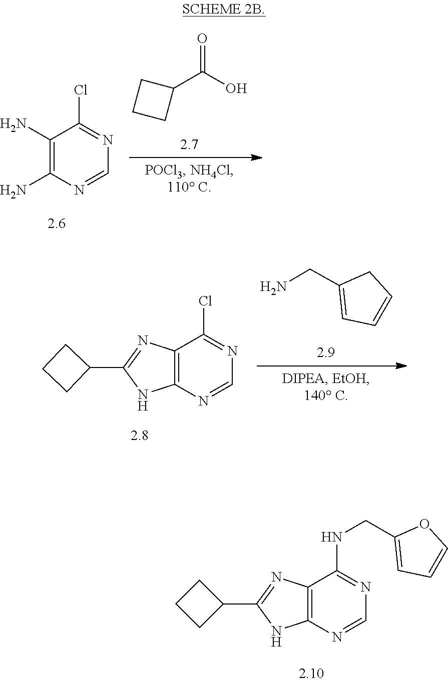

In some embodiments, compounds of type 2.5, and similar compounds, can be prepared according to reaction Scheme 2B above. Thus, compounds of type 2.8 can be prepared by a cyclization reaction of an appropriate diamine, e.g., 2.6 as shown above, and an appropriate carboxylic acid, e.g., 2.7 as shown above. Appropriate diamines and appropriate carboxylic acids are commercially available or prepared by methods known to one skilled in the art. The cyclization reaction is carried out in the presence of an appropriate oxidant, e.g., phosphorous oxychloride, and an appropriate base, e.g., ammonium chloride, at an appropriate temperature, e.g., 110° C. Compounds of type 2.10 can be prepared by a coupling reaction of an appropriate halide, e.g., 2.8 as shown above, and an appropriate amine, e.g., 2.9 as shown above. Appropriate amines are commercially available or prepared by methods known to one skilled in the art. The coupling reaction is carried out in the presence of an appropriate base, e.g., diisopropylethylamine (DIPEA), in an appropriate solvent, e.g., ethanol, at an appropriate temperature, e.g., 140° C. As can be appreciated by one skilled in the art, the above reaction provides an example of a generalized approach wherein compounds similar in structure to the specific reactants above (compounds similar to compounds of type 2.1, 2.2, 2.3, and 2.4), can be substituted in the reaction to provide substituted N-containing heteroaryl analogs similar to Formula 2.5.

### 3. Route III

In some embodiments, N-containing heteroaryl analogs can be prepared as shown below.

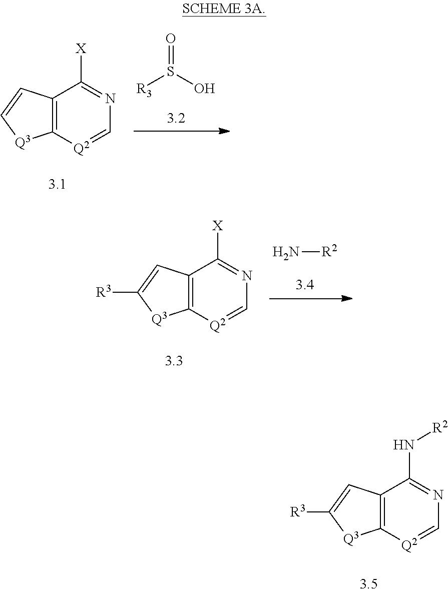

Compounds are represented in generic form, wherein Z is a halogen and with substituents as noted in compound descriptions elsewhere herein. A more specific example is set forth below.

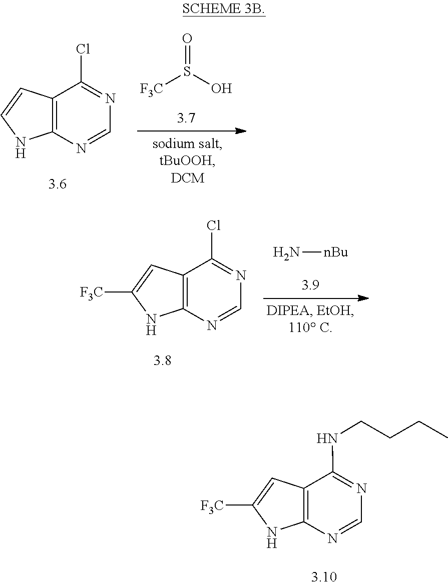

In some embodiments, compounds of type 3.5, and similar compounds, can be prepared according to reaction Scheme 3B above. Thus, compounds of type 3.3 can be prepared by a substitution reaction between an appropriate N-containing heteroaryl analog, e.g., 3.6 as shown above, and an appropriate sulfonic acid, e.g., 3.7 as shown above. Appropriate N-containing heteroaryl analogs and appropriate sulfonic acids are commercially available or prepared by methods known to one skilled in the art. The substitution reaction is carried out in the presence of an appropriate salt, e.g., a sodium salt, and an appropriate peroxide, e.g., tert-butyl hydrogen peroxide, in an appropriate solve, e.g., dichloromethane (DCM). Compounds of type 3.10 can be prepared by a coupling reaction of an appropriate halide, e.g., 3.8 as shown above, and an appropriate amine, e.g., 3.9 as shown above. Appropriate amines are commercially available or prepared by methods known to one skilled in the art. The coupling reaction is carried out in the presence of an appropriate base, e.g., diisopropylethylamine (DIPEA), in an appropriate solvent, e.g., ethanol, at an appropriate temperature, e.g., 110° C. As can be appreciated by one skilled in the art, the above reaction provides an example of a generalized approach wherein compounds similar in structure to the specific reactants above (compounds similar to compounds of type 3.1, 3.2, 3.3, and 3.4), can be substituted in the reaction to provide substituted N-containing heteroaryl analogs similar to Formula 3.5.

### 4. Route IV

In some embodiments, compounds can be prepared as shown below.

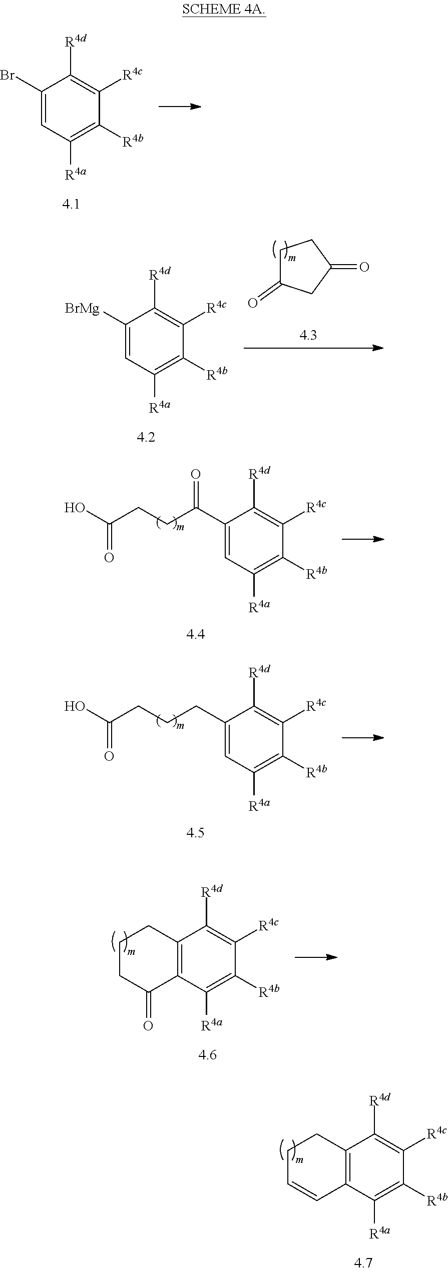

Compounds are represented in generic form, with substituents as noted in compound descriptions elsewhere herein. A more specific example is set forth below.

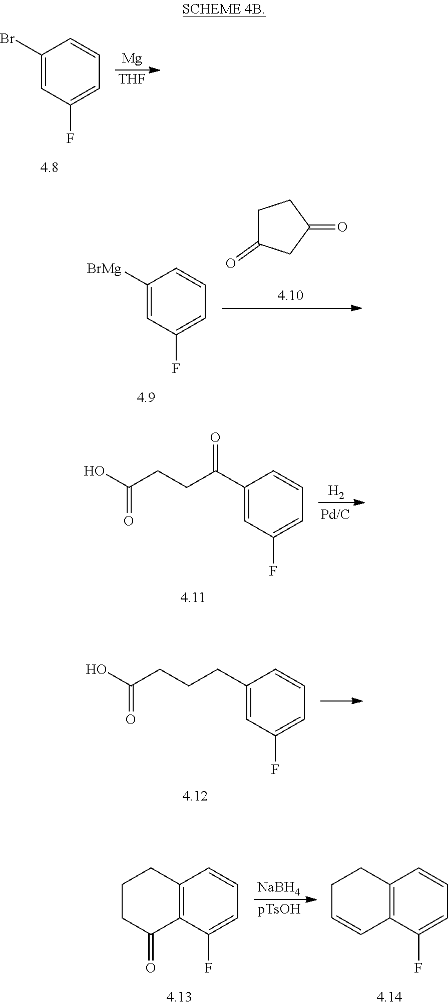

In some embodiments, compounds of type 4.14, and similar compounds, can be prepared according to reaction Scheme 4B above. Thus, compounds of type 4.11 can be prepared by a Grignard reaction of an appropriate aryl bromine, e.g., 4.8 as shown above. Appropriate aryl bromines are commercially available or prepared by methods known to one skilled in the art. The Grignard reaction is carried out in the presence of an appropriate metal source, e.g., magnesium metal, in an appropriate solvent, e.g., tetrahydrofuran (THF), followed by reaction with an appropriate carbonyl analog, e.g., 4.10 as shown above. Appropriate carbonyl analogs are commercially available or prepared by methods known to one skilled in the art. Compounds of type 4.12 can be prepared by reduction of an appropriate ketone, e.g., 4.11 as shown above. The reduction is carried out in the presence of an appropriate reducing agent, e.g., hydrogen gas, and an appropriate catalyst, e.g., palladium on carbon. Compounds of type 4.13 can be prepared by cyclization of an appropriate aryl carboxylic acid analog, e.g., 4.12 as shown above. The cyclization is carried out in the presence of strong acid (like trifluorosulfonic acid) and heat. Compounds of type 4.14 can be prepared by reduction of an appropriate ketone, e.g., 4.13 as shown above. The reduction is carried out in the presence of an appropriate activating agent, e.g., para-toluenesulfonic acid, and an appropriate reducing agent, e.g., sodium borohydride. As can be appreciated by one skilled in the art, the above reaction provides an example of a generalized approach wherein compounds similar in structure to the specific reactants above (compounds similar to compounds of type 4.1, 4.2, 4.3, 4.4, 4.5, and 4.6), can be substituted in the reaction to provide compounds similar to Formula 4.7.

### 5. Route V

In some embodiments, compounds can be prepared as shown below.

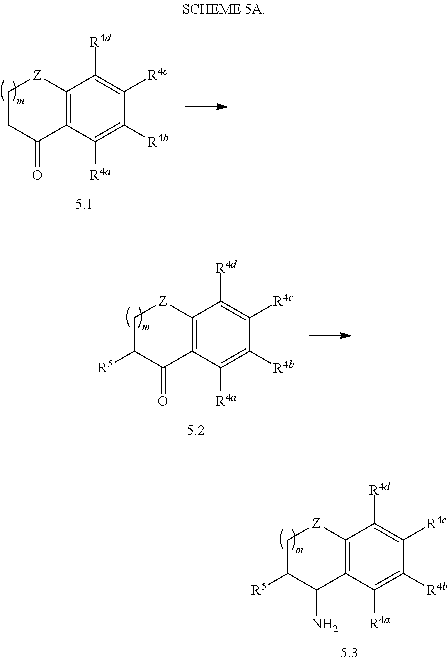

Compounds are represented in generic form, with substituents as noted in compound descriptions elsewhere herein. A more specific example is set forth below.

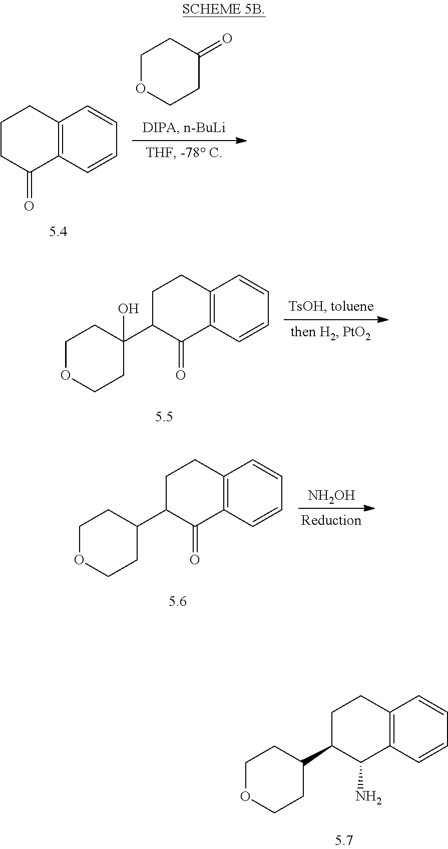

In some embodiments, compounds of type 5.7, and similar compounds, can be prepared according to reaction Scheme 5B above. Thus, compounds of type 5.5 can be prepared by reaction of an appropriate aryl ketone, e.g., 5.8 as shown above, and an appropriate ketone, e.g., tetrahydro-4H-pyran-4-one as shown above. Appropriate aryl ketones and appropriate ketones are commercially available or prepared by methods known to one skilled in the art. The reaction is carried out in the presence of an appropriate amine, e.g., diispropylamine (DIPA), and an appropriate base, e.g., n-butyl lithium, in an appropriate solvent, e.g., THF, at an appropriate temperature, e.g., −78° C. Compounds of type 5.6 can be prepared by reduction of an appropriate alcohol, e.g., 5.5 as shown above. The reduction is carried out in the presence of an appropriate activating agent, e.g., toluenesulfonic acid, in an appropriate solvent, e.g., toluene, followed by reaction with an appropriate reducing agent, e.g., hydrogen gas, and an appropriate catalyst, e.g., platinum oxide. Compounds of type 5.7 can be prepared by reductive amination of an appropriate ketone, e.g., 5.6 as shown above. The reductive amination is carried out in the presence of an appropriate agent, e.g., hydroxylamine. As can be appreciated by one skilled in the art, the above reaction provides an example of a generalized approach wherein compounds similar in structure to the specific reactants above (compounds similar to compounds of type 5.1 and 5.2), can be substituted in the reaction to provide compounds similar to Formula 5.3.

### 6. Route VI

In some embodiments, compounds can be prepared as shown below.

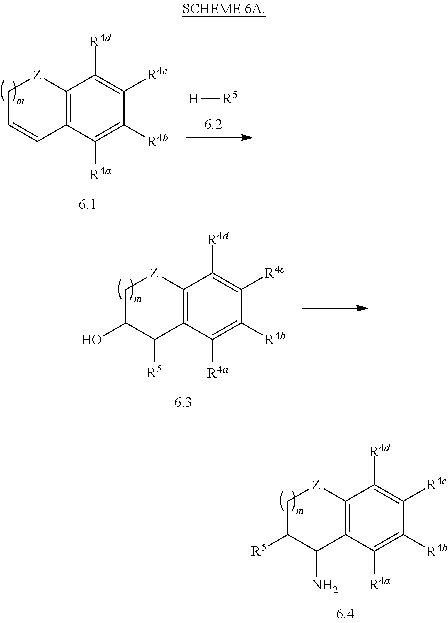

Compounds are represented in generic form, with substituents as noted in compound descriptions elsewhere herein. A more specific example is set forth below.

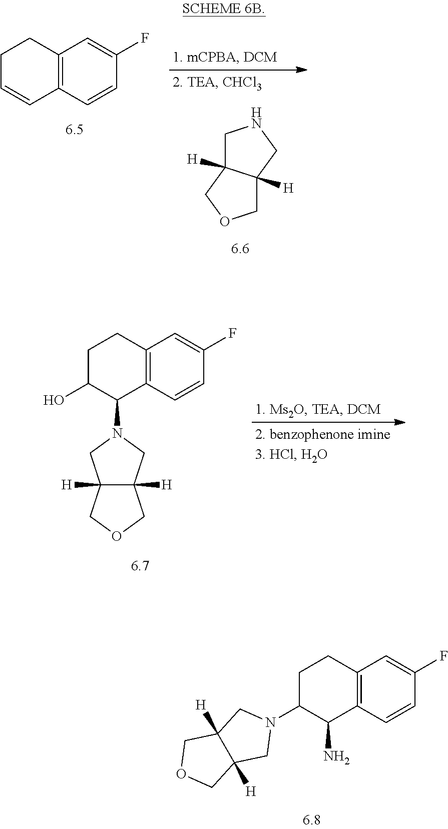 [SMILES: Fc1ccc2c(c1)CCC=C2 ; C1NC[C@H]2COC[C@@H]12 ; OC1CCc2cc(F)ccc2[C@H]1N1C[C@H]2COC[C@H]2C1 ; N[C@@H]1c2ccc(F)cc2CCC1N1C[C@H]2COC[C@H]2C1]

In some embodiments, compounds of type 6.8, and similar compounds, can be prepared according to reaction Scheme 6B above. Thus, compounds of type 6.7 can be prepared by epoxidation of an appropriate alkene, e.g., 6.5 as shown above. The epoxodation is carried out in the presence of an appropriate oxidizing agent, e.g., meta-chloroperoxybenzoic acid (mCPBA), in an appropriate solvent, e.g., dichloromethane (DCM), followed by ring opening in the presence of an appropriate amine, e.g., 6.6 as shown above. Appropriate amines are commercially available or prepared by methods known to those skilled in the art. The ring opening is carried out in the presence of an appropriate base, e.g., triethylamine (TEA), in an appropriate solvent, e.g., chloroform (CHCl₃). Compounds of type 6.8 can be prepared by rearrangement of an appropriate alcohol, e.g., 6.7 as shown above. The rearrangement is carried out in the presence of an appropriate activating agent, e.g., methanesulfonic anhydride, an appropriate base, e.g., TEA, in an appropriate solvent, e.g., DCM, followed by reaction with an appropriate imine, e.g., benzophenone imine. As can be appreciated by one skilled in the art, the above reaction provides an example of a generalized approach wherein compounds similar in structure to the specific reactants above (compounds similar to compounds of type 6.1, 6.2, and 6.3), can be substituted in the reaction to provide compounds similar to Formula 6.4.

### 7. Route VII

In some embodiments, compounds can be prepared as shown below.

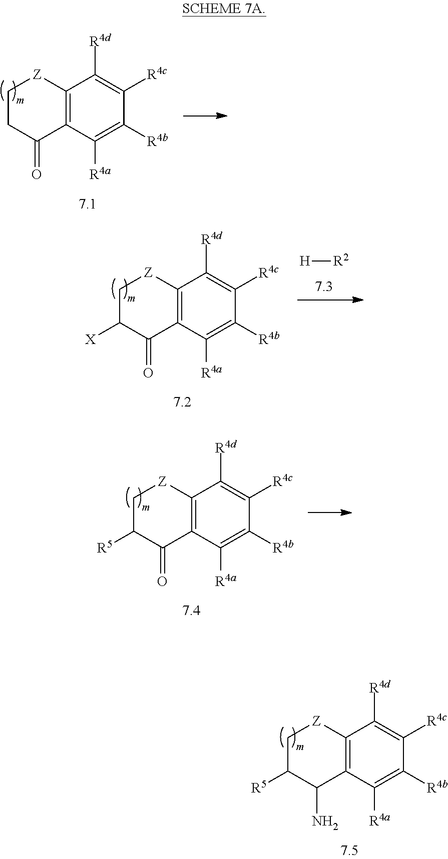

Compounds are represented in generic form, with substituents as noted in compound descriptions elsewhere herein. A more specific example is set forth below.

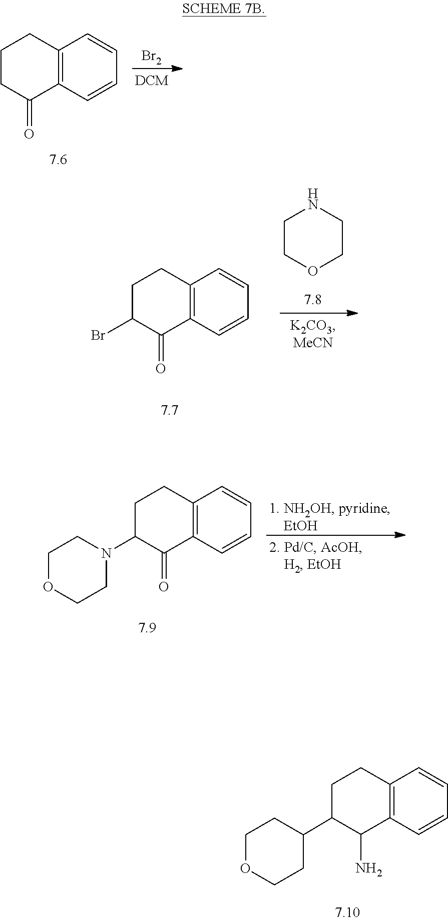

In some embodiments, compounds of type 7.10, and similar compounds, can be prepared according to reaction Scheme 7B above. Thus, compounds of type 7.7 can be prepared by halogenation of an appropriate ketone, e.g., 7.6 as shown above. Appropriate ketones are commercially available or prepared by methods known to one skilled in the art. The halogenation is carried out in the presence of an appropriate halide source, e.g., bromine, in an appropriate solvent, e.g., DCM. Compounds of type 7.9 can be prepared by displacement of an appropriate halide, e.g., 7.7 as shown above. The displacement reaction is carried out in the presence of an appropriate nucleophilic agent, e.g., 7.8 as shown above, and an appropriate base, e.g., potassium carbonate, in an appropriate solvent, e.g., acetonitrile. Appropriate nucleophilic agents are commercially available or prepared by methods known to one skilled in the art. Compounds of type 7.10 can be prepared by reductive amination of an appropriate ketone, e.g., 7.9 as shown above. The reductive amination is carried out in the presence of an appropriate amine, e.g., hydroxylamine, and an appropriate base, e.g., pyridine, in an appropriate solvent, e.g., ethanol, followed by reaction with an appropriate reducing agent, e.g., hydrogen gas, an appropriate catalyst, e.g., palladium on carbon, and an appropriate acid, e.g., acetic acid, in an appropriate solvent, e.g., ethanol. As can be appreciated by one skilled in the art, the above reaction provides an example of a generalized approach wherein compounds similar in structure to the specific reactants above (compounds similar to compounds of type 7.1, 7.2, 7.3, and 7.4), can be substituted in the reaction to provide compounds similar to Formula 7.5.

### 8. Route VIII

In some embodiments, N-containing heteroaryl analogs can be prepared as shown below.

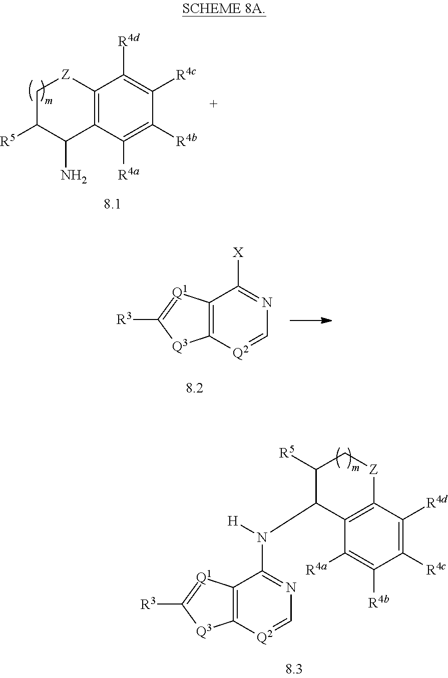

Compounds are represented in generic form, wherein X is halogen and with other substituents as noted in compound descriptions elsewhere herein. A more specific example is set forth below.

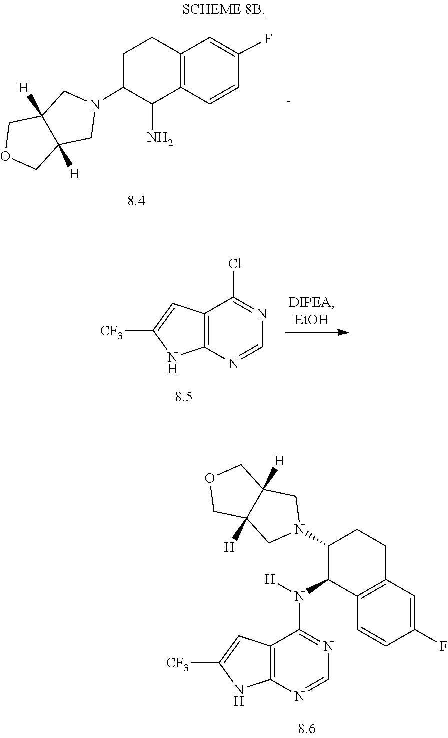 [SMILES: NC1c2ccc(F)cc2CCC1N1C[C@H]2COC[C@H]2C1 ; FC(F)(F)c1cc2c(Cl)ncnc2[nH]1 ; Fc1ccc2c(c1)CC[C@@H](N1C[C@H]3COC[C@H]3C1)[C@@H]2Nc1ncnc2[nH]c(C(F)(F)F)cc12]

In some embodiments, compounds of type 8.6, and similar compounds, can be prepared according to reaction Scheme 8B above. Thus, compounds of type 8.6 can be prepared by arylation of an appropriate amine, e.g., 8.4 as shown above. The arylation is carried out in the presence of an appropriate halide, e.g., 8.5 as shown above, and an appropriate base, e.g., diisopropylethyl amine (DIPEA), in an appropriate solvent, e.g., ethanol (EtOH). Appropriate halides are commercially available or prepared by methods known to those skilled in the art. As can be appreciated by one skilled in the art, the above reaction provides an example of a generalized approach wherein compounds similar in structure to the specific reactants above (compounds similar to compounds of type 8.1 and 8.2), can be substituted in the reaction to provide N-containing heteroaryl analogs similar to Formula 8.3.

Compounds and compositions described herein are generally useful for modulating the activity of PINK1. In some embodiments, the compounds and compositions described herein inhibit the activity of PINK1. In some embodiments, the disclosure relates to a method of modulating PINK1 in a subject in need thereof or in a cell. The disclosure relates to a method of modulating PINK1 in a subject in need thereof comprising administering to the subject a pharmaceutically effective amount of one or a plurality of compositions comprising a compound disclosed herein or a derivative or salt thereof. In some embodiments, the disclosure relates to a method of modulating PINK1 in a cell comprising contacting the cell with one or a plurality of compositions, each composition comprising a pharmaceutically effective amount of one or a plurality of compounds disclosed herein or a derivative or salt thereof. Method of PINK1 anti-receptor effective amount. In some embodiments, the disclosure relates to a method of modulating PINK1 in a cell comprising contacting the cell with one or a plurality of compositions, each composition comprising an anti-receptor effective amount of one or a plurality of compounds disclosed herein or a derivative or salt thereof. In some embodiments, the cell is in a healthy subject or a subject in need of treatment for one of the indications disclosed herein. In embodiments, the method of modulating PINK1 activity is an in vitro method of modulating activity of a cell in tissue culture.

### D. Methods of Treating a Disorder in a Subject

In some embodiments, compounds and compositions described herein are useful in treating a disorder associated with PINK1, such as, in some embodiments, a kidney disease, a fibrotic disorder, a cisplatin-induced toxicity or a reperfusion injury. Thus, provided herein are methods of treating a disorder associated with PINK1 function, comprising administering to a subject in need thereof, a therapeutically effective amount of a compound described herein, or a pharmaceutically acceptable salt thereof, or a composition comprising a disclosed compound or pharmaceutically acceptable salt thereof. Disorders treatable by the present compounds and compositions include, e.g., a kidney disease such as, for example, chronic kidney disease or acute kidney injury (AKI); fibrotic disorders such as, for example, pulmonary fibrosis, liver fibrosis, heart fibrosis, mediastinal fibrosis, retroperitoneal cavity fibrosis, bone marrow fibrosis, skin fibrosis, scleroderma, pancreatic fibrillation, prostatic hyperplasia caused by fibrillation, and renal fibrosis; and reperfusion injuries such as, for example, reperfusion injuries induced by a mitochondrial disease (e.g., myocardial ischemia or stroke caused by Mitochondrial Encephalopathy, Lactic Acidosis, and Stroke-like episodes (MELAS)) and reperfusion injuries that are not induced by a mitochondrial disease (e.g., transplantation reperfusion, hepatic ischemia reperfusion, renal ischemia reperfusion, cerebral ischemia reperfusion). In some embodiments, the disclosure relates to a method of treating a cisplatin-induced toxicity, such as ototoxicity, comprising administering a pharmaceutically effective amount of the disclosed compounds or salts or derivative thereof.

In some embodiments, the disclosure relates to any of the above disclosed methods disclosed herein, wherein the administrating step comprises administering a pharmaceutical composition comprising: (i) a pharmaceutically effective amount of any of the disclosed compounds or a pharmaceutically acceptable salt thereof; and (ii) a pharmaceutically acceptable carrier.

Thus, in various embodiments, disclosed are methods of treating a disorder in a subject in need thereof comprising administering to the subject an effective amount of a disclosed compound. In some embodiments, the compound has a structure represented by a formula:

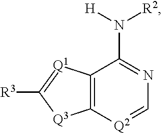

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; wherein R¹ is selected from C1-C6 haloalkyl, C1-C6 haloalkoxy, C1-C6 halohydroxyalkyl, and a structure represented by a formula:

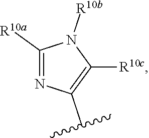

wherein each of R^{10a}, R^{10b}, and R^{10c}, when present, is independently selected from hydrogen and C1-C4 alkyl; wherein Q² is N or CH; wherein Q³ is CH₂ or NH; wherein R² is selected from C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy²; wherein n, when present, is 0, 1, or 2; wherein R¹², when present, is selected from hydrogen and C1-C4 alkyl; wherein Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and provided that when Q¹ is CR¹, R¹ is C1-C6 haloalkyl, and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl, or a pharmaceutically acceptable salt thereof, wherein the disorder is a kidney disease, a fibrotic disorder, or a reperfusion injury.

In some embodiments, the compound has a structure represented by a formula:

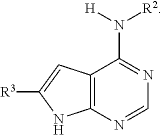

In some embodiments, the compound has a structure represented by a formula:

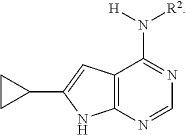

In some embodiments, the compound has a structure represented by a formula:

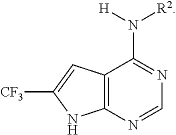

In some embodiments, the compound has a structure represented by a formula:

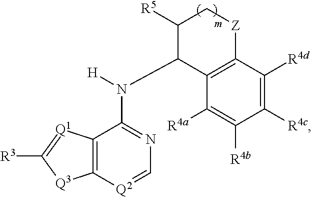

wherein m is 0 or 1; wherein Q¹ is N or CH and R³ is a 3- to 6-membered cycloalkyl, C1-C6 haloalkyl, C1-C6 haloalkoxy, or C1-C6 halohydroxyalkyl; wherein Z is CR^{13a}R^{13b} or O; wherein each of R^{13a} and R^{13b}, when present, is independently selected from hydrogen, halogen, —OH, and C1-C4 alkoxy, or wherein each of R^{13a} and R^{13b}, when present, together comprise ═O; wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R⁵ is selected from —O(CH₂)ₙCy², —NR¹²(CH₂)ₙCy², and Cy².

In some embodiments, the compound is selected from:

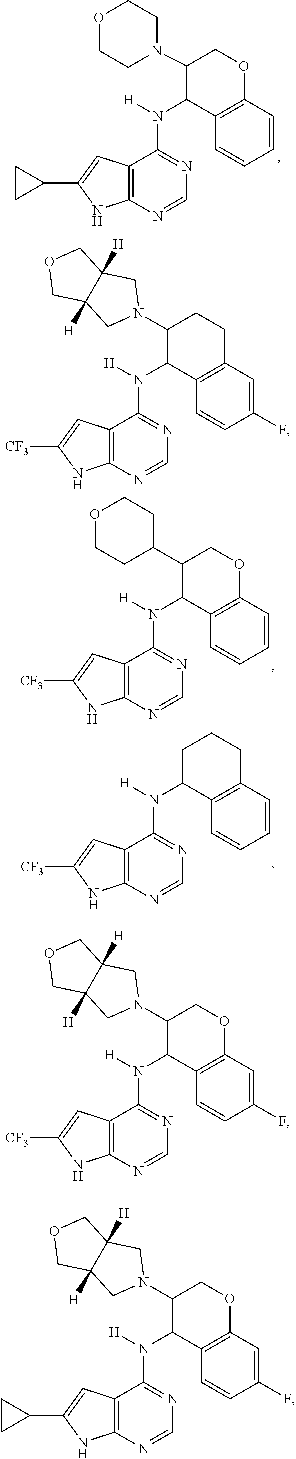

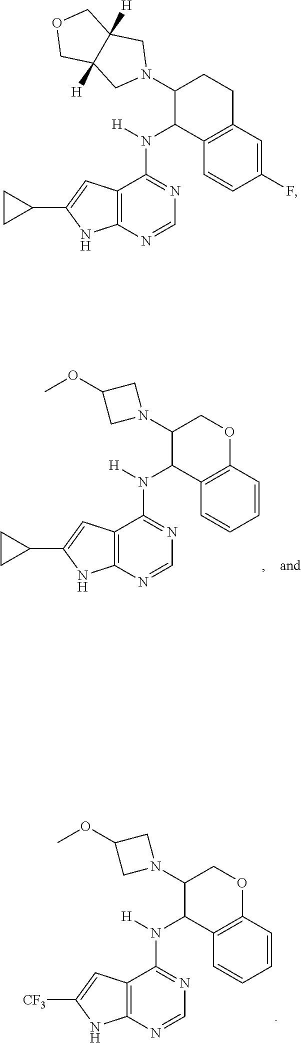 [SMILES: Fc1ccc2c(c1)CCC(N1C[C@H]3COC[C@H]3C1)C2Nc1ncnc2[nH]c(C3CC3)cc12 ; COC1CN(C2COc3ccccc3C2Nc2ncnc3[nH]c(C4CC4)cc23)C1 ; COC1CN(C2COc3ccccc3C2Nc2ncnc3[nH]c(C(F)(F)F)cc23)C1]

In some embodiments, the compound is selected from:

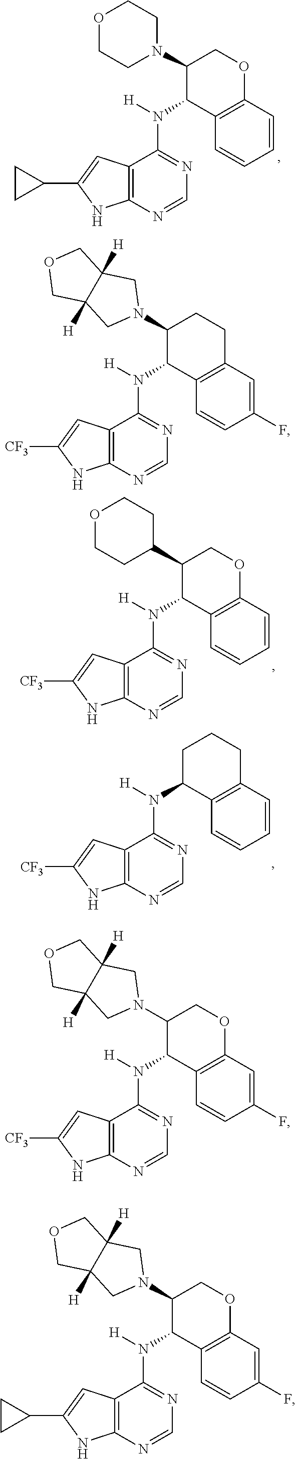

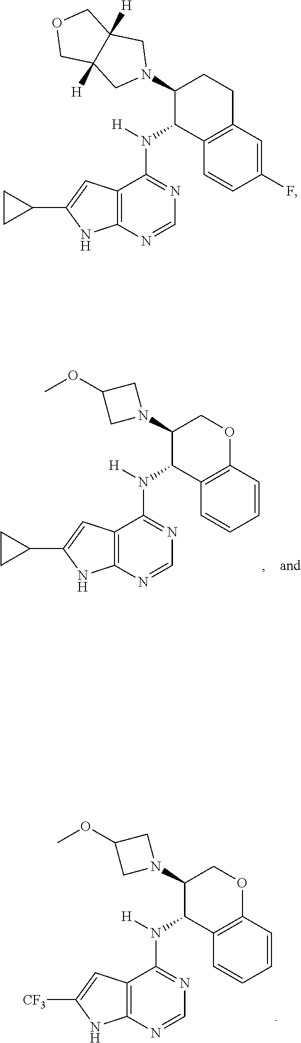 [SMILES: Fc1ccc2c(c1)CC[C@H](N1C[C@H]3COC[C@H]3C1)[C@H]2Nc1ncnc2[nH]c(C3CC3)cc12 ; COC1CN([C@H]2COc3ccccc3[C@@H]2Nc2ncnc3[nH]c(C4CC4)cc23)C1 ; COC1CN([C@H]2COc3ccccc3[C@@H]2Nc2ncnc3[nH]c(C(F)(F)F)cc23)C1]

In some embodiments, the compound is selected from:

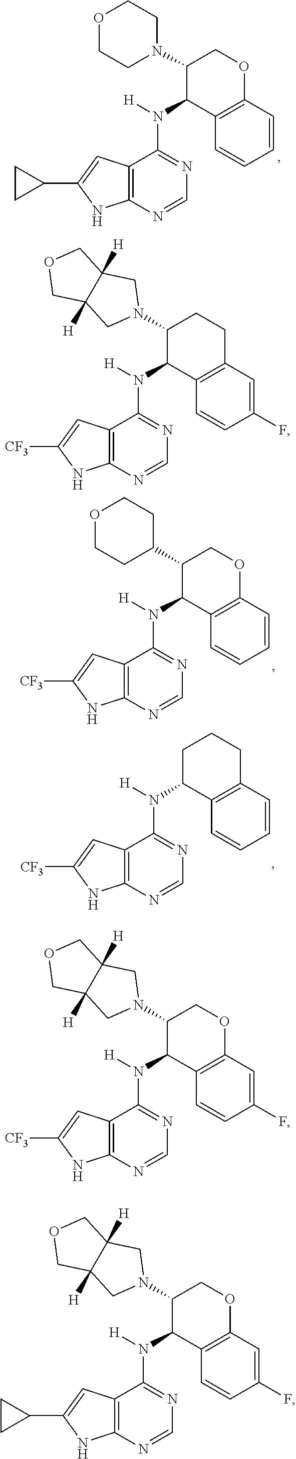

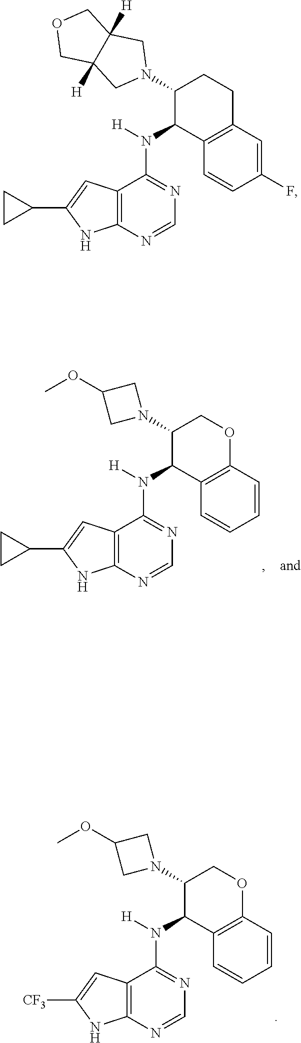 [SMILES: Fc1ccc2c(c1)CC[C@@H](N1C[C@H]3COC[C@H]3C1)[C@@H]2Nc1ncnc2[nH]c(C3CC3)cc12 ; COC1CN([C@@H]2COc3ccccc3[C@H]2Nc2ncnc3[nH]c(C4CC4)cc23)C1 ; COC1CN([C@@H]2COc3ccccc3[C@H]2Nc2ncnc3[nH]c(C(F)(F)F)cc23)C1]

Examples of kidney diseases that may be treated with a compound or composition described herein include chronic kidney disease (e.g., autosomal dominant polycystic kidney disease, diabetic nephropathy, hypertension-induced renal injury, crescentic glomerulonephritis, membranous nephropathy, membranous nephropathy, IgA nephropathy, amyloid A amyloidosis, secondary nephrotic syndrome) or acute kidney injury (AKI).

Examples of fibrotic disorders that may be treated with a compound or composition described herein include pulmonary fibrosis, liver fibrosis, heart fibrosis, mediastinal fibrosis, retroperitoneal cavity fibrosis, bone marrow fibrosis, skin fibrosis, scleroderma, pancreatic fibrillation, prostatic hyperplasia caused by fibrillation, and renal fibrosis.

Examples of reperfusion injuries that may be treated with a compound or composition described herein include reperfusion injuries induced by a mitochondrial disease (e.g., myocardial ischemia or stroke caused by Mitochondrial Encephalopathy, Lactic Acidosis, and Stroke-like episodes (MELAS)) and reperfusion injuries that are not induced by a mitochondrial disease (e.g., transplantation reperfusion, hepatic ischemia reperfusion, renal ischemia reperfusion, cerebral ischemia reperfusion).

In certain embodiments, the disease treated by a disclosed compound or composition is one that is characterized by an increase in the level of PINK1. In certain embodiments, the disease is one characterized by neural cell death. In certain embodiments, the disease is one characterized by an increase in the level of PINK1 activity. In certain embodiments, the disease is a kidney disease. In certain embodiments, the disease is a fibrotic disorder. In certain embodiments, the disease is a reperfusion injury.

In further embodiments, the subject has been diagnosed with a need for treatment of a disorder associated with PINK1 kinase activity prior to the administering step.

In further embodiments, the subject is a mammal. In still further embodiments, the mammal is a human.

In further embodiments, the method further comprises the step of identifying a subject in need of treatment of a disorder associated with PINK1 kinase activity.

In further embodiments, the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

In further embodiments, effective amount is a therapeutically effective amount. In still further embodiments, wherein the effective amount is a prophylactically effective amount.

In various embodiments, the method further comprises administering an effective amount of an agent associated with the treatment of a kidney disease, a fibrotic disorder, or a reperfusion injury. In some embodiments, the disclosure relates to a method of preventing reperfusion injury or cisplatin-induced toxicity, such as ototoxicity, comprising administering to a subject a pharmaceutical composition comprising a pharmaceutically effective amount of compound disclosed herein or a pharmaceutically acceptable salt or derivative thereof. In some embodiments, the compound is a prodrug of a compound disclosed herein. In some embodiments, the disclosure related to a method of reducing cisplatin-reduced toxicity in a subject comprising administering to a subject a pharmaceutical composition comprising a pharmaceutically effective amount of compound disclosed herein or a pharmaceutically acceptable salt or derivative thereof. In some embodiments, the subject is a patient who is receiving cisplatin as a treatment for another illness, such as cancer. In some embodiments, the subject is a human patient who is taking or has taken cisplatin as a treatment. In some embodiments, the subject is a human patient diagnosed with cisplatin-sensitive cancer. In some embodiments, the disclosure relates to a method of preventing reperfusion injury or fibrosis in a subject susceptible to reperfusion injury or fibrosis comprising administering to the subject a pharmaceutical composition comprising a pharmaceutically effective amount of compound disclosed herein or a pharmaceutically acceptable salt or derivative thereof. In some embodiments, the method of prevention comprises administering to a human subject that has kidney damage or is susceptible to kidney damage or a human subject that has been prescribed cisplatin for a cisplating-sensitive cancer.

Thus, in various embodiments, the method further comprises administering an agent associated with the treatment of a kidney disease or a fibrotic disorder. Examples of agents associated with the treatment of a kidney disease or a fibrotic disorder include, but are not limited to an angiotensin-converting enzyme (ACE) inhibitors (e.g., benazepril, captopril, enalapril, fosinopril, lisinopril, moexipril, perindopril, quinapril, Ramipril, trandolapril), an angiotensin II receptor blockers (e.g., azilsartan, candesartan, eprosartan, irbesartan, losartan, olmesartan, telmisartan, valsartan), nintedanib, pirfenidone, autotaxin inhibitors, and peroxisome proliferator-activated receptor (PPAR) modulators (e.g., ADGE, EPI-001, INT-131, K-0533, S26948).

In various embodiments, the method further comprises administering an agent associated with the treatment of a reperfusion injury. Examples of agents associated with the treatment of a reperfusion injury include, but are not limited to, hydrogen sulfide, cyclosporine, TRO40303, superoxide dismutase, metformin, elamipretide, and cannabinoids.

In some embodiments, the compound and the agent are administered simultaneously.

In some embodiments, the compound and the agent are administered sequentially.

### E. Kits

In some embodiments, disclosed are kits comprising a disclosed compound, or a pharmaceutically acceptable salt thereof, and one or more of: (a) an agent associated with the treatment of a kidney disease or a fibrotic disorder; (b) an agent associated with the treatment of a reperfusion injury; (c) instructions for administering the compound in connection with treating a kidney disease, a fibrotic disorder, and/or a reperfusion injury; and (d) instructions for treating a kidney disease, a fibrotic disorder, and/or a reperfusion injury.

In some embodiments, disclosed are kits comprising a compound having a structure represented by a formula:

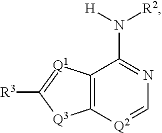

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; wherein R¹ is selected from C1-C6 haloalkyl, C1-C6 haloalkoxy, C1-C6 halohydroxyalkyl, and a structure represented by a formula:

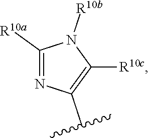

wherein each of R^{10a}, R^{10b}, and R^{10c}, when present, is independently selected from hydrogen and C1-C4 alkyl; wherein Q² is N or CH; wherein Q³ is CH₂ or NH; wherein R² is selected from C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy²; wherein n, when present, is 0, 1, or 2; wherein R¹², when present, is selected from hydrogen and C1-C4 alkyl; wherein Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and provided that when Q¹ is CR¹, R¹ is C1-C6 haloalkyl, and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b} when present, is hydrogen, and Cy^{I} is a 6-membered aryl or furanyl, then Q¹ is CH and R³ is not a C1-C6 haloalkyl, or a pharmaceutically acceptable salt thereof, and one or more of: (a) an agent associated with the treatment of a kidney disease or a fibrotic disorder; (b) an agent associated with the treatment of a reperfusion injury; (c) instructions for administering the compound in connection with treating a kidney disease, a fibrotic disorder, and/or a reperfusion injury; and (d) instructions for treating a kidney disease, a fibrotic disorder, and/or a reperfusion injury.

In some embodiments, the compound has a structure represented by a formula:

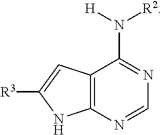

In some embodiments, the compound has a structure represented by a formula:

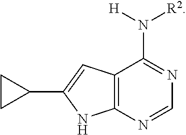

In some embodiments, the compound has a structure represented by a formula:

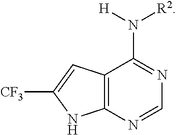

In some embodiments, the compound has a structure represented by a formula:

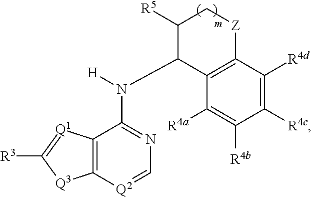

wherein m is 0 or 1; wherein Q¹ is N or CH and R³ is a 3- to 6-membered cycloalkyl, C1-C6 haloalkyl, C1-C6 haloalkoxy, or C1-C6 halohydroxyalkyl; wherein Z is CR^{13a}R^{13b} or O; wherein each of R^{13a} and R^{13b}, when present, is independently selected from hydrogen, halogen, —OH, and C1-C4 alkoxy, or wherein each of R^{13a} and R^{13b}, when present, together comprise ═O; wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R⁵ is selected from —O(CH₂)ₙCy², —NR¹²(CH₂)ₙCy², and Cy².

In some embodiments, the compound is selected from:

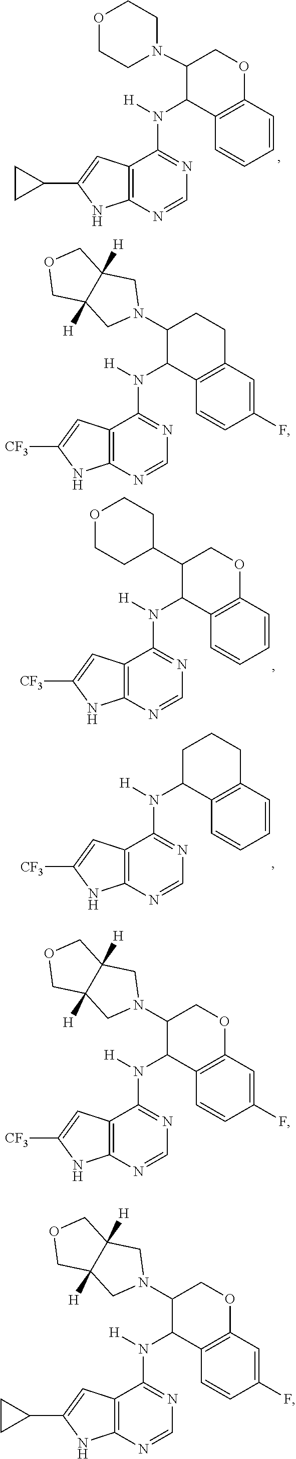

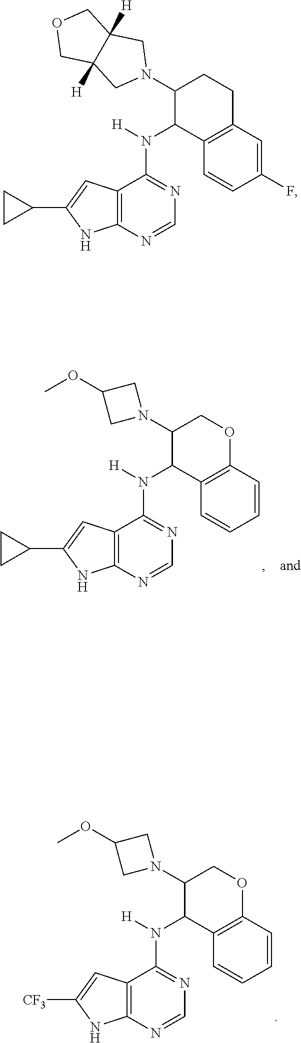 [SMILES: Fc1ccc2c(c1)CCC(N1C[C@H]3COC[C@H]3C1)C2Nc1ncnc2[nH]c(C3CC3)cc12 ; COC1CN(C2COc3ccccc3C2Nc2ncnc3[nH]c(C4CC4)cc23)C1 ; COC1CN(C2COc3ccccc3C2Nc2ncnc3[nH]c(C(F)(F)F)cc23)C1]

In some embodiments, the compound is selected from:

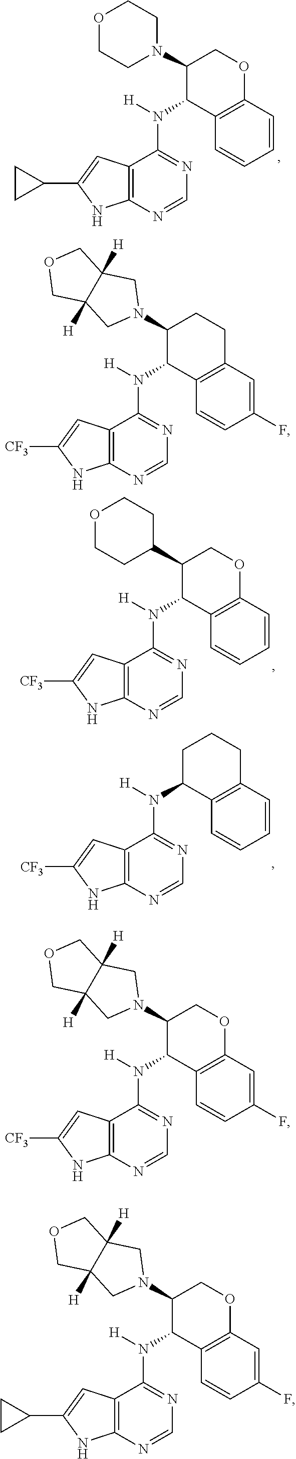

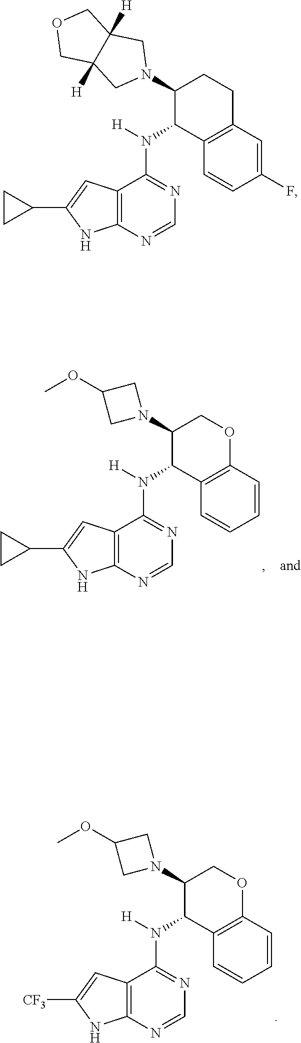 [SMILES: Fc1ccc2c(c1)CC[C@H](N1C[C@H]3COC[C@H]3C1)[C@H]2Nc1ncnc2[nH]c(C3CC3)cc12 ; COC1CN([C@H]2COc3ccccc3[C@@H]2Nc2ncnc3[nH]c(C4CC4)cc23)C1 ; COC1CN([C@H]2COc3ccccc3[C@@H]2Nc2ncnc3[nH]c(C(F)(F)F)cc23)C1]

In some embodiments, the compound is selected from:

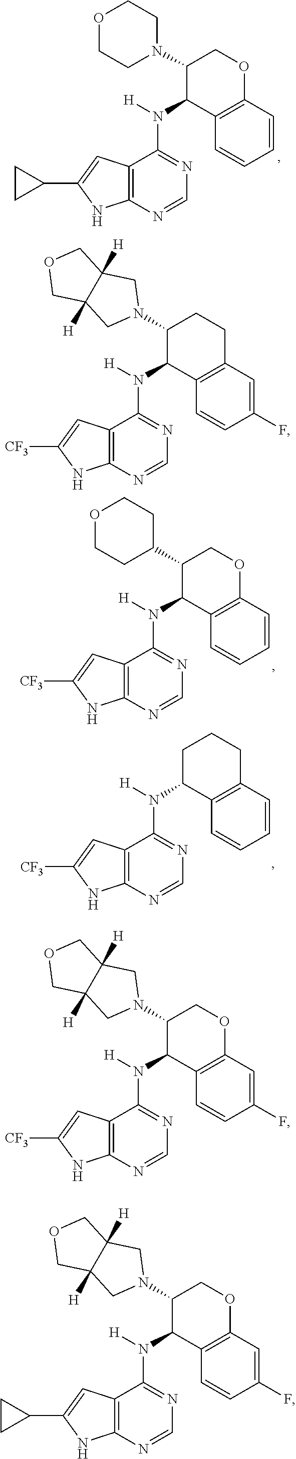

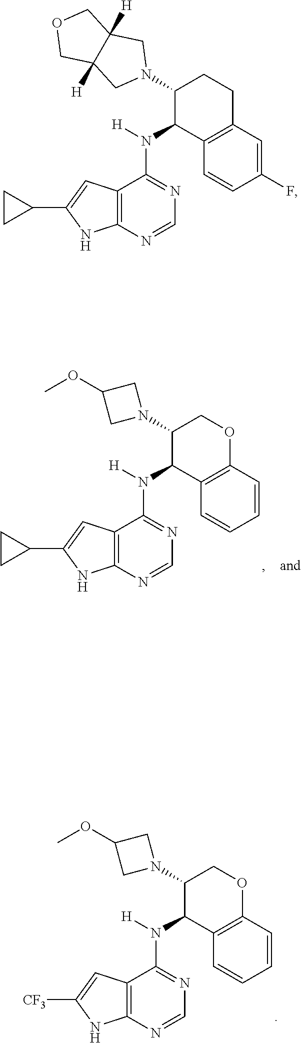 [SMILES: Fc1ccc2c(c1)CC[C@@H](N1C[C@H]3COC[C@H]3C1)[C@@H]2Nc1ncnc2[nH]c(C3CC3)cc12 ; COC1CN([C@@H]2COc3ccccc3[C@H]2Nc2ncnc3[nH]c(C4CC4)cc23)C1 ; COC1CN([C@@H]2COc3ccccc3[C@H]2Nc2ncnc3[nH]c(C(F)(F)F)cc23)C1]

In further embodiments, the agent is known for the treatment of fibrosis such as, for example, idiopathic pulmonary fibrosis (IPF), non-alcoholic fatty liver disease (NASH), liver fibrosis, heart fibrosis, mediastinal fibrosis, bone marrow fibrosis, retroperitoneal cavity fibrosis, and renal fibrosis. Examples of agents known for the treatment of fibrosis include, but are not limited to, pirfenidone, nintedanib, a prostaglandin such as latanoprost and bimaotoprost, a beta blocker such as timolol and betaxolol, an alpha-adrenergic agonist such as apraclonidine and brimonidine, a carbonic anhydrase inhibitor such as dorzolamide and brinzolamide, a moitic or cholinergic agent such as pilocarpine, a diuretic, an angiotenisin-converting enzyme (ACE) inhibitor, an angiotensin II receptor blocker, an anti-inflammatory agent, and an anti-fibrotic agent.

In further embodiments, the agent is known for the treatment of a kidney disease or a fibrotic disorder. Examples of agents known for the treatment of a kidney disease or a fibrotic disorder include, but are not limited to an angiotensin-converting enzyme (ACE) inhibitors (e.g., benazepril, captopril, enalapril, fosinopril, lisinopril, moexipril, perindopril, quinapril, Ramipril, trandolapril), an angiotensin II receptor blockers (e.g., azilsartan, candesartan, eprosartan, irbesartan, losartan, olmesartan, telmisartan, valsartan), nintedanib, pirfenidone, autotaxin inhibitors, and peroxisome proliferator-activated receptor (PPAR) modulators (e.g., ADGE, EPI-001, INT-131, K-0533, S26948, ASP1128).

In further embodiments, the agent is known for the treatment of a reperfusion injury. Examples of agents known for the treatment of a reperfusion injury include, but are not limited to, hydrogen sulfide, cyclosporine, TRO40303, superoxide dismutase, metformin, elamipretide, and cannabinoids.

In further embodiments, the at least one compound and the at least one agent are co-formulated. In further embodiments, the at least one compound and the at least one agent are co-packaged.

In further embodiments, the compound and the agent associated with the treatment of a kidney disease or a fibrotic disorder are co-packaged. In still further embodiments, the compound and the agent associated with the treatment of a kidney disease or a fibrotic disorder are co-formulated.

In further embodiments, the compound and the agent associated with the treatment of a reperfusion injury are co-packaged. In still further embodiments, the compound and the agent associated with the treatment of a reperfusion injury are co-formulated.

In further embodiments, the compound and the agent are administered sequentially. In still further embodiments, the compound and the agent are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the disclosure concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the disclosure to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Examples

Representative examples of the disclosed compounds are illustrated in the following non-limiting methods, schemes, and examples.

### 1. Biology Protocols

a. In-Life Procedures Cisplatin Challenge and Dosing Regimen

Mice were provided at least one week of acclimation to the animal facility and group housed. Mice were injected intraperitoneally with 1 mg/ml cisplatin solution (BluePoint Labs) or 10 ml/kg sterile-filtered saline using 29 G insulin syringes. Mice were weighed and administered vehicle, 35985 or 40180 by oral gavage per the dosing regimens noted in the figures. Mice were monitored for excessive weight loss and euthanized if moribund.

### b. 35985 and 40180 Formulation

35985 and 40180 are formulated at ten-fold the dosing concentration in NMP (N-methylpyrrolidone) followed by dilution with solutol-15 and water for a final vehicle concentration of 10% NMP/10% solutol-15/80% water.

c. Sacrifice and Tissue Collection and Storage

For tissue harvest, mice were anesthetized using isofluorane. Cardiac puncture was performed to withdraw blood for serum collection. Blood was deposited into serum separator tubes and left undisturbed for 30 min to 1 hr at room temperature to allow clotting prior to serum separation by centrifugation for 2 min (10,000 g, room temperature). Collected serum was transferred to Eppendorf tubes and frozen on dry ice. After cervical dislocation, left and right kidneys were extracted and frozen until analysis.

d. Kidney Homogenate Preparation and Mitochondrial Isolation

Kidneys were removed from −80° C. and minced on an ice block. Minced tissues were transferred to a dounce homogenizer and homogenized with 20× strokes of the ‘loose’ pestle and 20× strokes of the ‘tight’ pestle using 1 ml ul of cold mitochondrial isolation buffer (MIB, 50 mM Tris-HCl (pH 7.5), 70 mM sucrose, 210 mM sorbitol, 1 mM EDTA, 1 mM EGTA, 100 mM chloroacetamide, Halt™ Protease and Phosphatase Inhibitor Cocktail, EDTA-free (100×) (PI), 10 μM PR619). Kidney homogenate was transferred to a 1.5 ml Eppendorf tube and were centrifuged at 300×g for 5 min at 4° C. Approximately 800 ul of supernatant was transferred to a new 1.5 ml microcentrifuge tube. The supernatant (cytosol+mitochondria) was transferred to a new tube and centrifuged at 10,000 g for 20 min at 4° C. to pellet the mitochondrial fraction. After removing residual supernatant, mitochondria were resuspended in lysis buffer (100 mM Bicine pH 8.0, 0.27M Sucrose, 1 mM EDTA, 1 mM EGTA, 5 mM Na4P2O7, 100 mM Tris pH 7.5, 1% Triton X-100), containing benzonase (1:1000), HALT protease/phosphatase inhibitors (1:100), and PR-619 de-ubiquitinase inhibitor (1:1000).

e. Blood Urea Nitrogen (BUN) Determination

Serum was thawed on ice and subsequently diluted 1:50 in MilliQ water. BUN levels in the serum sample were analyzed using ThermoFisher's Urea Nitrogen (BUN) Colorimetric Detection Kit. Assay was performed following manufacturer's published protocol.

f. Kidney Injury Molecule 1 (KIM-1) Determination

Urine was collected from scruffed mice (serial collection) or directly from bladder using insulin syringe during harvest (terminal collection). KIM-1 was measured in mouse urine using R&D System's Mouse TIM-1/KIM-1/HAVCR DuoSet ELISA following manufacturer's published protocol.

g. Kidney RNA Extraction and Quantitative PCR

RNA was isolated from kidney samples using Rneasy Mini kit (Qiagen) according to its product manual. RNA concentration was measured using NanoDrop™ 2000/2000c Spectrophotometers (Thermo Scientific). 50 ng of RNA for each sample was used to generate cDNA. cDNA was synthesized using High-Capacity RNA-to-cDNA™ Kit (Thermo Scientific) according to its product manual. Quantitative PCR was performed using Power SYBR™ Green PCR Master Mix (Applied Biosystems) according to its product manual. The following primers used to analyze gene expression levels in the kidney: Tnfrsf12a; 5′-GTGTTGGGATTCGGCTTGGT-3′ (SEQ ID NO:1) and 5′-GTCCATGCACTTGTCGAGGTC-3′ (SEQ ID NO:2), Atf3; 5′-GAGGATTTTGCTAACCTGACACC-3′ (SEQ ID NO:3) and 5′-TTGACGGTAACTGACTCCAGC-3′ (SEQ ID NO:4), Plk3; 5′-GCACATCCATCGGTCATCCAG-3′ (SEQ ID NO: 5) and 5′-GCCACAGTCAAACCTTCTTCAA-3′ (SEQ ID NO:6), Gdf15; 5′-CTGGCAATGCCTGAACAACG-3′ (SEQ ID NO:7) and 5′-GGTC GGGACTTGGTTCTGAG-3′ (SEQ ID NO:8), b-act; 5′-GGGCATCCTGACCCTC AAG-3′ (SEQ ID NO:9) and 5′-TCCATGTCGTCCCAGTTGGT-3′ (SEQ ID NO:10). All gene expression levels were normalized to expression levels of beta-actin using ΔΔCt and expressed as fold change relative to cisplatin vehicle treated mice.

h. mtDNA/nucDNA Ratio

A small piece of frozen kidney tissue (˜12 mg) was homogenized and DNA extracted using the Qiagen QIAamp DNA mini kit. mtDNA/nucDNA ratio was determined using a qPCR protocol from the Aurwex lab (Quiros et al, 2017), using the following primers: 16S rRNA 5′-CCGCAAGGGAAAGATGAAAGAC-3′(SEQ ID NO:11) and 5′-TCGTTTGGTTTCGGGGTTTC-3′ (SEQ ID NO:12); ND1 5′-CTAGCAGAAACAAACCGGGC-3′ (SEQ ID NO: 13) and: 5′-CCGGCTGCGTATTCTACGTT-3 (SEQ ID NO:14); HK2 5′-GCCAGCCTCTCCTGATTTTAGTGT-3′ (SEQ ID NO:15) and 5′-GGGAACACAAAAGACCTCTTCTGG-3′ (SEQ ID NO:16).

i. pS65-Ub ELISA

For pS65-Ub ELISA, capture monoclonal rabbit antibody anti-pS65-Ub was diluted to 1 μg/ml in PBS and pipetted into 96 well half-area polystyrene plates (50 ul/well). Sealed plates were shaken at 800 rpm for 5 minutes and incubated overnight at 4° C. on an even surface. The next day, blocking solution (5% BSA in TBST, sterile filtered) was added to each well (100 ul/well) and shaken for 1 hr at 800 rpm at RT. Plates were either used immediately or stored sealed at 4° C. for maximum one week. Samples were diluted in lysis buffer to a concentration of 10 ug/ul and 50 ul were loaded onto plates in duplicate after washing 5× with TBST using an automated plate washer (used for all subsequent wash steps). Standard protein recombinant pS65-Ub was diluted in lysis buffer+0.1% BSA and serial dilutions (4000 ng/ml-0 ng/ml) were added in duplicate to the sample plate (50 ul/well). Plates were shaken at 800 rpm at RT for 2 hr. After washing 5× with TBST, 50 ul of mouse anti-Ub detection antibody (1 ug/ml in 5% BSA in TBST) was added to the wells. Plates were shaken at 800 rpm at RT for 1 hr, followed by washing 5× with TBST, and shaking at 800 rpm at RT for 45 minutes with goat anti-mouse peroxidase-conjugated IgG antibody (1:10,000 dilution in 5% BSA in TBST) (50 ul/well). For peroxidase reaction, 50 ul of TMB reagent (Pierce #34029) was added to the wells after washing and wells were monitored closely for reaction development. To stop the ELISA reaction, 50 ul 2N sulfuric acid was added. Absorbance was measured at 450 nm using LifeTechnologies SpectraMax).

j. Western Blotting

The total protein concentration of kidney mitopreps was measured with the Thermo Scientific Pierce BCA Protein Assay Kit (Thermo Scientific), according to its product manual. These samples were normalized with their respective lysis buffers. For SDS-PAGE, the samples were prepared with 4× Laemmli Sample Buffer with the reducing agent 2 mercaptoethanol. For each lane of a 26 well gel (4-20% Criterion™ Tris-HCl Protein Gel, Bio-Rad Laboratories), 10 μg per sample was loaded and analyzed by Western Blotting. Indicated bands were quantified using ImageStudio Lite and normalized to beta actin band intensity.

### 2. Summary of N-Containing Heteroaryl Analogs Evaluated for PINK1 Kinase Activity

A list of exemplary compounds is shown in Table 1 below.

TABLE 1

| No. | Structure |
|---|---|
| 35985 | 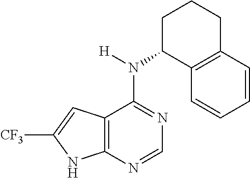 [SMILES: FC(F)(F)c1cc2c(N[C@@H]3CCCc4ccccc43)ncnc2[nH]1] |
| 40180 | 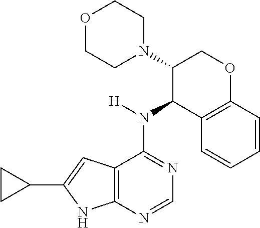 [SMILES: c1ccc2c(c1)OC[C@@H](N1CCOCC1)[C@@H]2Nc1ncnc2[nH]c(C3CC3)cc12] |
| 40695 | 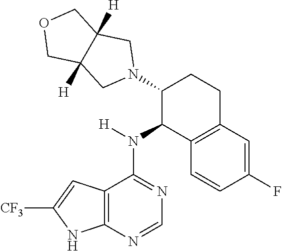 [SMILES: Fc1ccc2c(c1)CC[C@@H](N1C[C@H]3COC[C@H]3C1)[C@@H]2Nc1ncnc2[nH]c(C(F)(F)F)cc12] |
| 40700 | 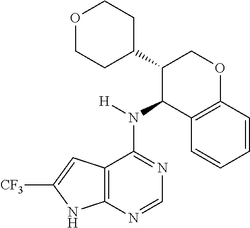 [SMILES: FC(F)(F)c1cc2c(N[C@@H]3c4ccccc4OC[C@H]3C3CCOCC3)ncnc2[nH]1] |
| 40748 | 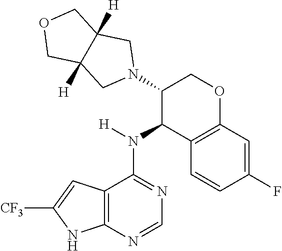 [SMILES: Fc1ccc2c(c1)OC[C@@H](N1C[C@H]3COC[C@H]3C1)[C@@H]2Nc1ncnc2[nH]c(C(F)(F)F)cc12] |
| 40644 | 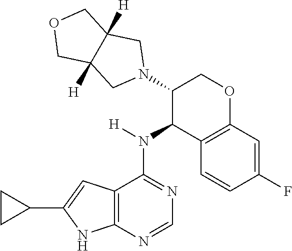 [SMILES: Fc1ccc2c(c1)OC[C@@H](N1C[C@H]3COC[C@H]3C1)[C@@H]2Nc1ncnc2[nH]c(C3CC3)cc12] |
| 40669 | 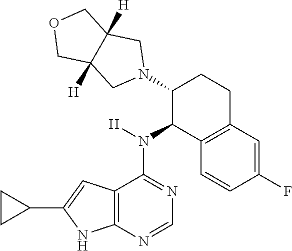 [SMILES: Fc1ccc2c(c1)CC[C@@H](N1C[C@H]3COC[C@H]3C1)[C@@H]2Nc1ncnc2[nH]c(C3CC3)cc12] |
| 40848 | 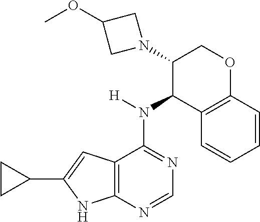 [SMILES: COC1CN([C@@H]2COc3ccccc3[C@H]2Nc2ncnc3[nH]c(C4CC4)cc23)C1] |
| 40850 | 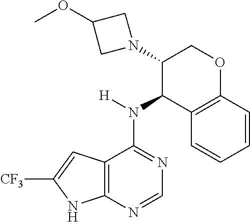 [SMILES: COC1CN([C@@H]2COc3ccccc3[C@H]2Nc2ncnc3[nH]c(C(F)(F)F)cc23)C1] |
| 40270 | 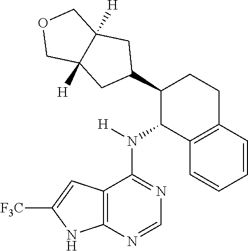 [SMILES: FC(F)(F)c1cc2c(N[C@H]3c4ccccc4CC[C@@H]3C3C[C@@H]4COC[C@H]4C3)ncnc2[nH]1] |
| 40949 | 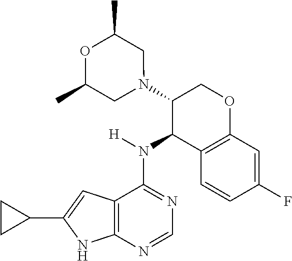 [SMILES: C[C@@H]1CN([C@@H]2COc3cc(F)ccc3[C@H]2Nc2ncnc3[nH]c(C4CC4)cc23)C[C@H](C)O1] |
| 41088 | 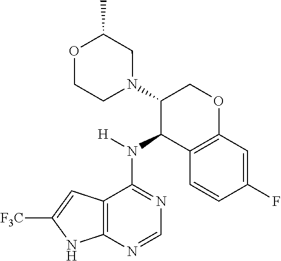 [SMILES: C[C@@H]1CN([C@@H]2COc3cc(F)ccc3[C@H]2Nc2ncnc3[nH]c(C(F)(F)F)cc23)CCO1] |

### 3. In Vitro Data

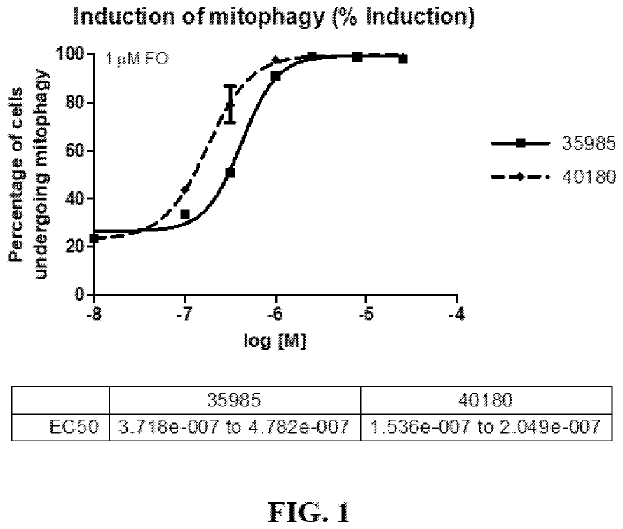

As shown in FIG. 1, the addition of 35985 or 40180 results in a dose responsive increase in the percentage of cells undergoing mitophagy. Briefly, HeLa cells expressing a mitophagy indicator (mtKeima) protein were treated with 1 mM of FCCP and Oligomycin followed by the specified dose of compound, then analyzed by FACS to quantify the percentage of cells undergoing mitophagy.

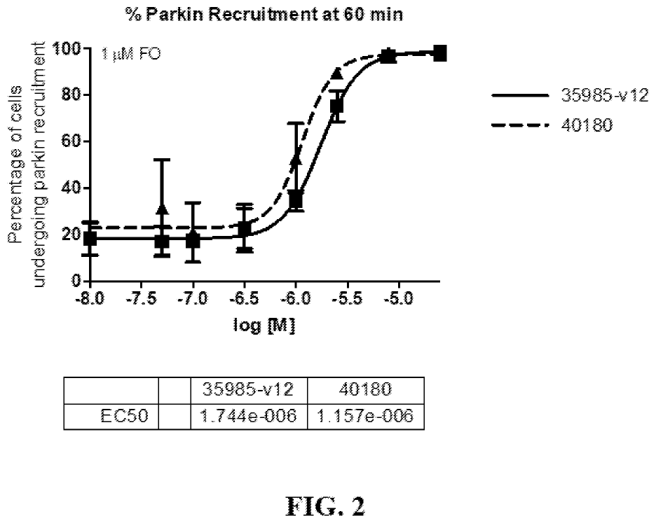

As shown in FIG. 2, addition of 35985 or 40180 results in a dose responsive increase in the rate of Parkin recruitment to mitochondria. HeLa cells expressing a YFP-tagged Parkin were treated with 1 mM of FCCP and Oligomycin followed by the specified dose of compound, then analyzed by longitudinal imaging. Percentage of cells with Parkin recruited to mitochondria at 60 minutes is shown.

### 4. Pathway Engagement Data

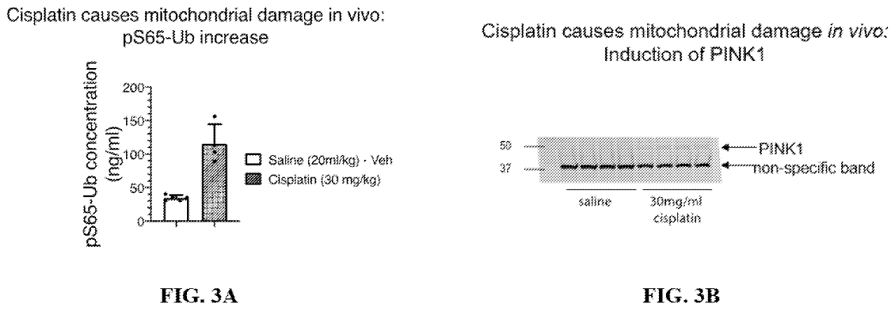

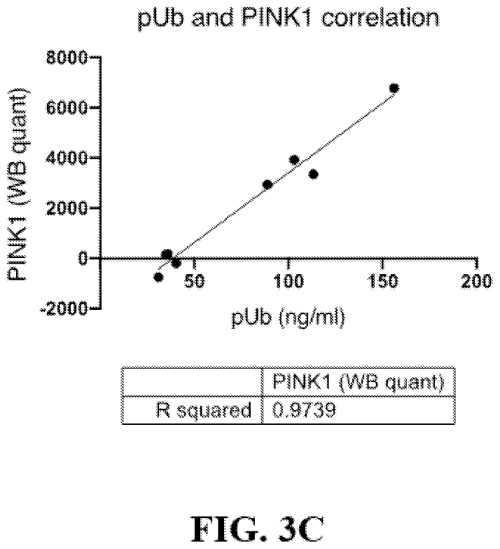

Mice (C57Bl/6) were challenged with a single intraperitoneal dose of 30 mg/kg cisplatin. Mitochondrial preparations were examined using pS65-Ub ELISA (FIG. 3A) or Western blot for PINK1 (FIG. 3B). Referring to FIG. 3C, there is a high degree of correlation between PINK1 protein concentration and its direct target, pS65-Ub, in kidney mitochondria.

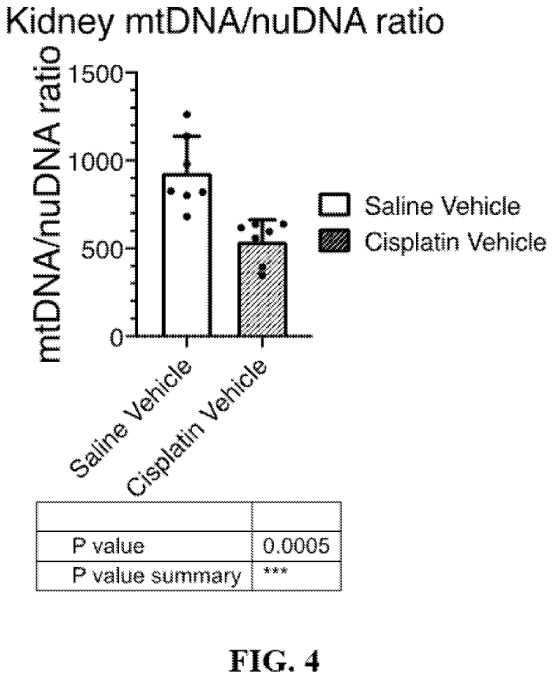

Referring to FIG. 4, mice (C57Bl/6) were challenged with a single intraperitoneal dose of 10 mg/kg cisplatin. Tissue lysates were examined for mitochondrial gene ND1 and nuclear gene beta actin by quantitative PCR. A significant reduction in mitochondrial DNA was observed upon cisplatin challenge.

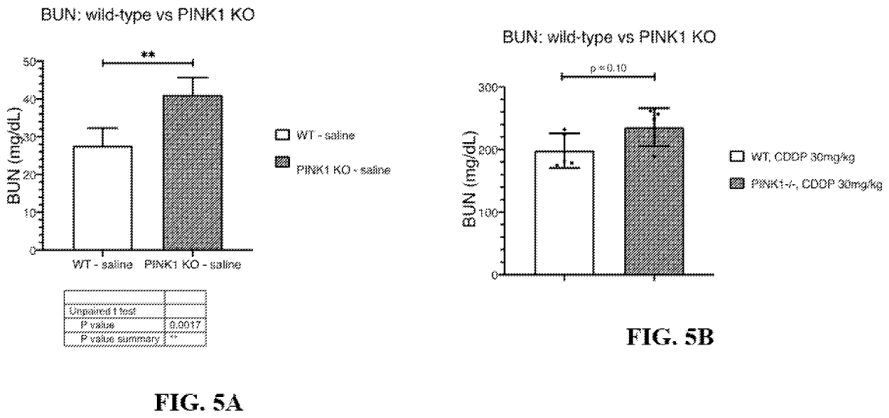

Referring to FIG. 5A and FIG. 5B, mice (C57Bl/6 or PINK1 knockout in C57Bl/6 background) were challenged with a single dose of saline or 30 mg/kg cisplatin (intraperitoneal). Blood urea nitrogen (BUN), a commonly used clinical marker for kidney dysfunction, is increased in the PINK1 knockout mice relative to the wild-type mice.

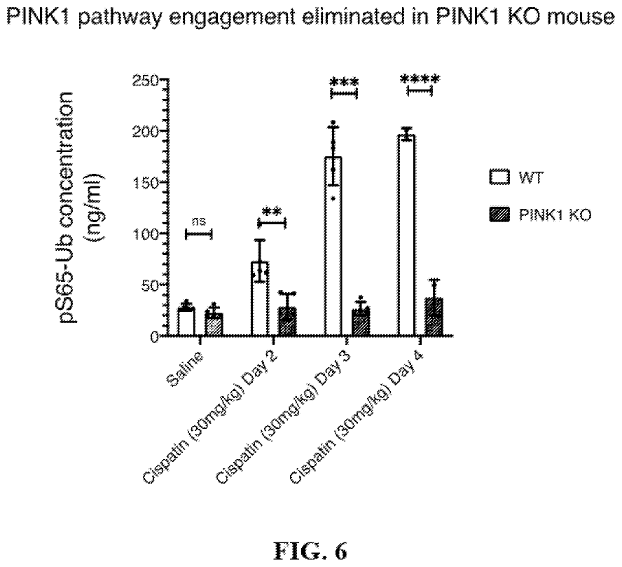

Referring to FIG. 6, mice (C57Bl/6 or PINK1 knockout in C57Bl/6 background) were challenged with a single intraperitoneal dose of 30 mg/kg cisplatin. Kidney mitochondria pS65-Ub levels were examined after different times as indicated. The fact that pS65 ubiquitin remains unchanged in PINK KO mice after cisplatin challenge indicates that PINK1 function is completely eliminated in those animals.

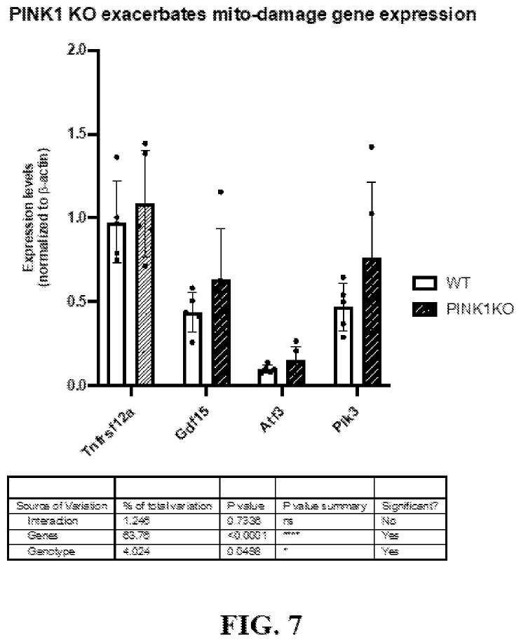

Referring to FIG. 7, mice (C57Bl/6 or PINK1 knockout in C57Bl/6 background) were challenged with a single intraperitoneal dose of 30 mg/kg cisplatin. Gene expression levels in the kidney for mitochondrial stress-responsive genes were examined using quantitative PCR. Mitochondrial stress gene expression is significantly increased in PINK1 knockout mice.

### 5. PK Data

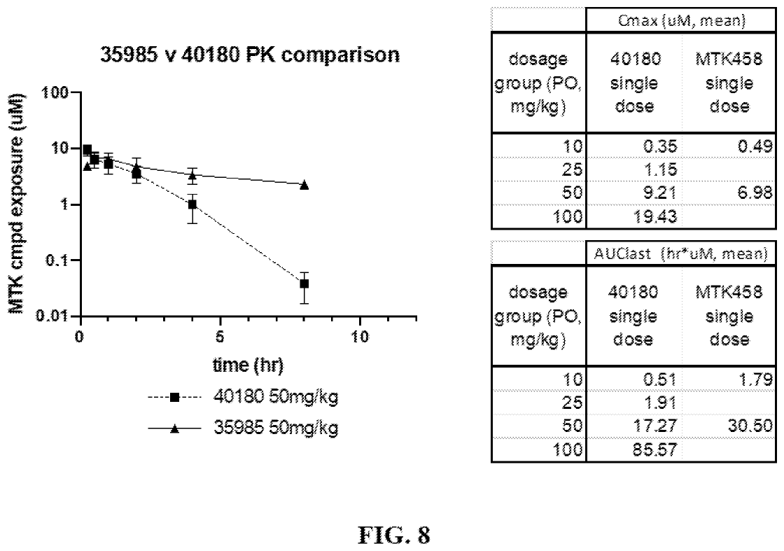

Briefly, mice (C57Bl/6, fed) were dosed by oral gavage with either 35985 or 40180 in NMP/solutol vehicle. Plasma concentrations of 35985 or 40180 were determined by mass spectrometry in at least 3 mice per study. Data for 50 mg/kg dose-level is shown in the graph of FIG. 8; computed pharmacokinetic parameters for different dose-levels are illustrated in the tables.

### 6. 35985 In Vivo Data

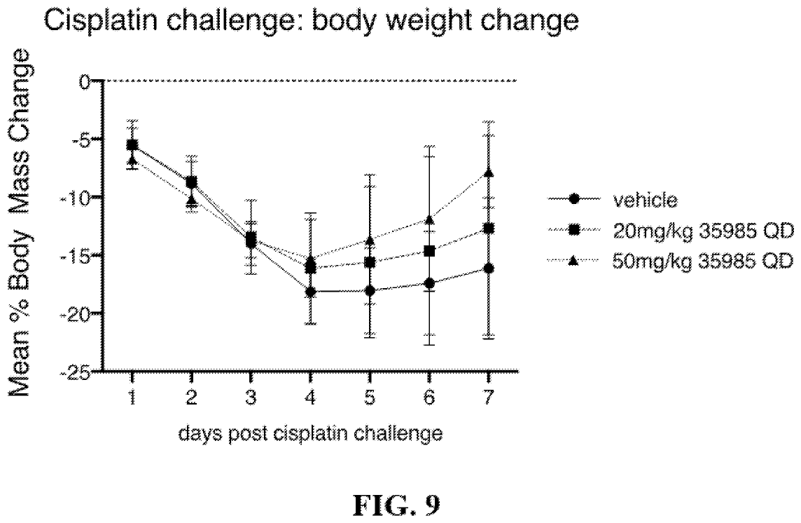

Referring to FIG. 9, mice (C57Bl/6) were challenged with a single intraperitoneal dose of 10 mg/kg cisplatin or saline (not shown) and dosed by oral gavage once per day (QD) with 35985. 35985 demonstrated a dose-dependent rescue of cisplatin-mediated weight-loss.

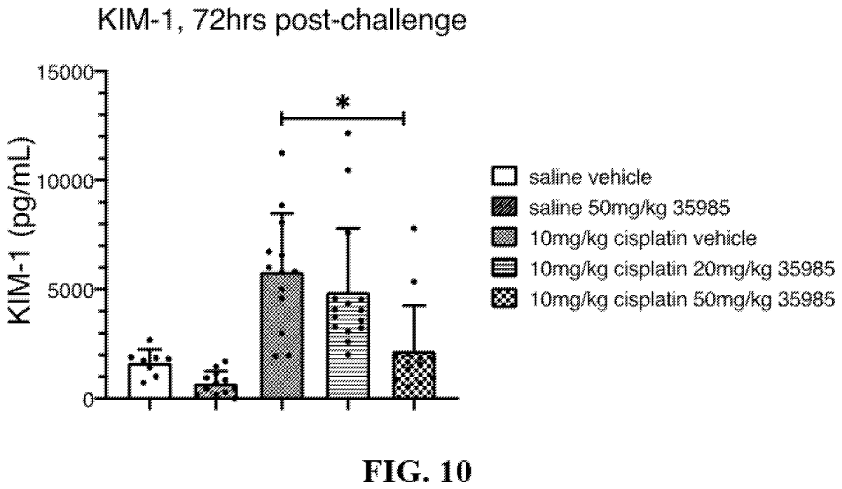

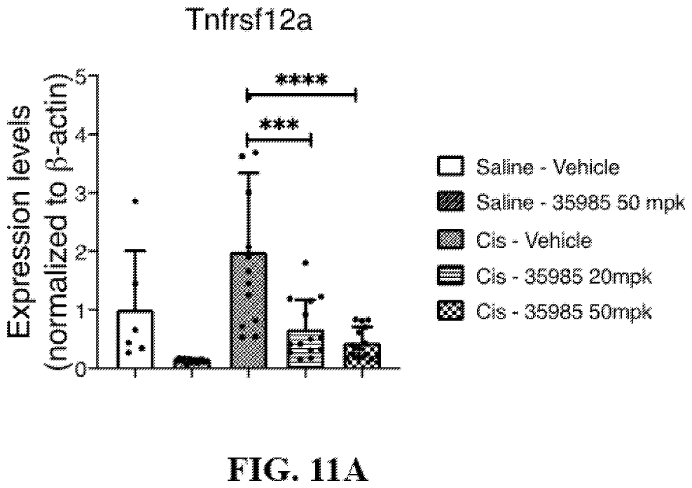

Referring to FIG. 10, mice (C57Bl/6) were challenged with a single intraperitoneal dose of 10 mg/kg cisplatin or saline (not shown) and dosed by oral gavage once per day (QD) with 35985. On day 3 post-challenge, 35985 demonstrated a dose-dependent rescue of KIM-1 (kidney-injury marker 1), a urine biomarker specific for kidney injury.

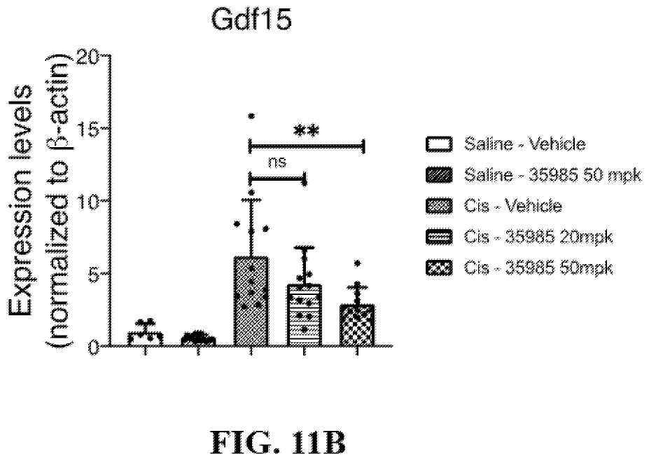

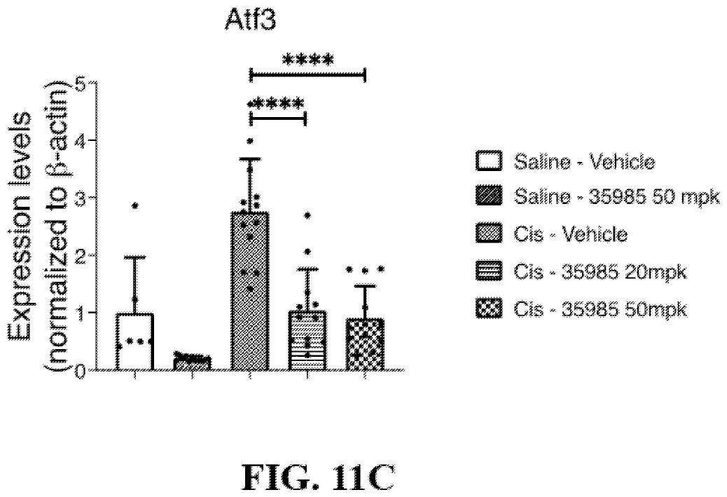

I.P. injection of 10 mg/kg cisplatin induces mitochondrial damage that drives an increase in mitochondrial stress markers Tnfrs12a (FIG. 11A), Gdf15 (FIG. 11B), and Atf3 (FIG. 11C). Oral dosing of 35985 at 20 or 50 mg/kg for seven days reduces the expression of all three genes as quantified by qPCR. *** p<0.0001, ** p<0.01.

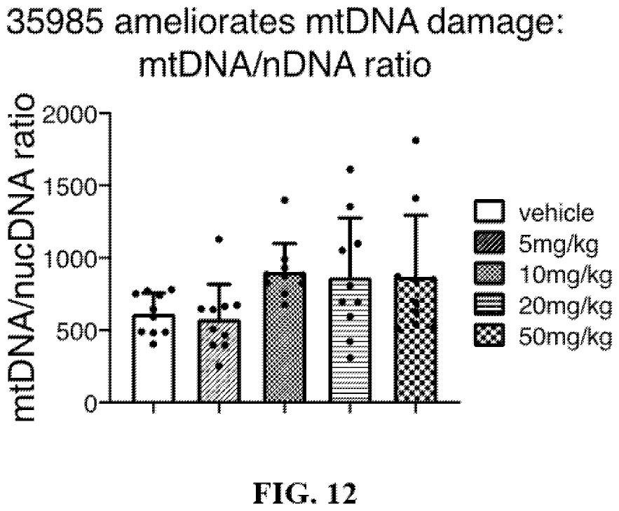

Referring to FIG. 12, mice (C57Bl/6) were challenged with a single intraperitoneal dose of 10 mg/kg cisplatin and dosed by oral gavage, once per day (QD) for seven days with 35985. 35985 at 10 mg/kg and above rescued cisplatin-mediated mitochondrial DNA loss.

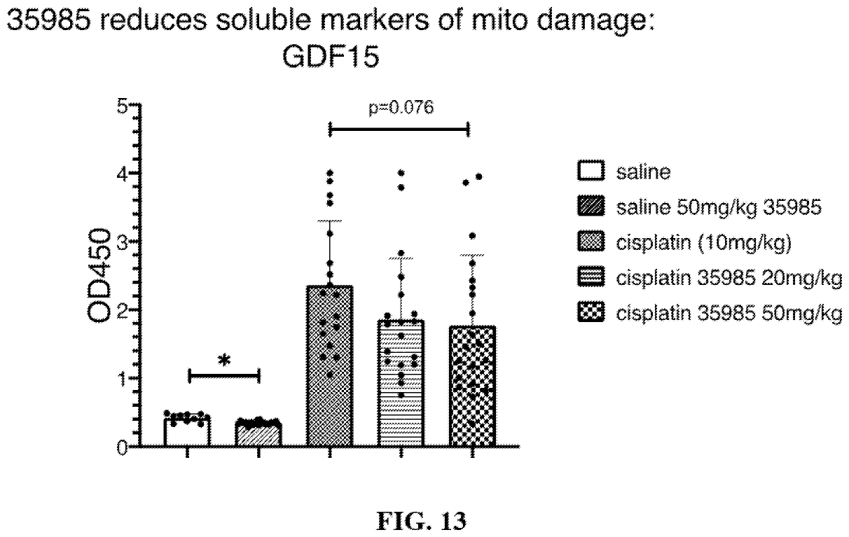

Referring to FIG. 13, mice (C57Bl/6) were challenged with a single intraperitoneal dose of 10 mg/kg cisplatin or saline and dosed by oral gavage once per day (QD) with 20 mg/kg or 50 mg/kg 35985. 35985 treatment reduced mitochondrial-stress serum biomarker GDF15 after 7 days.

### 7. 40180 In Vivo Data

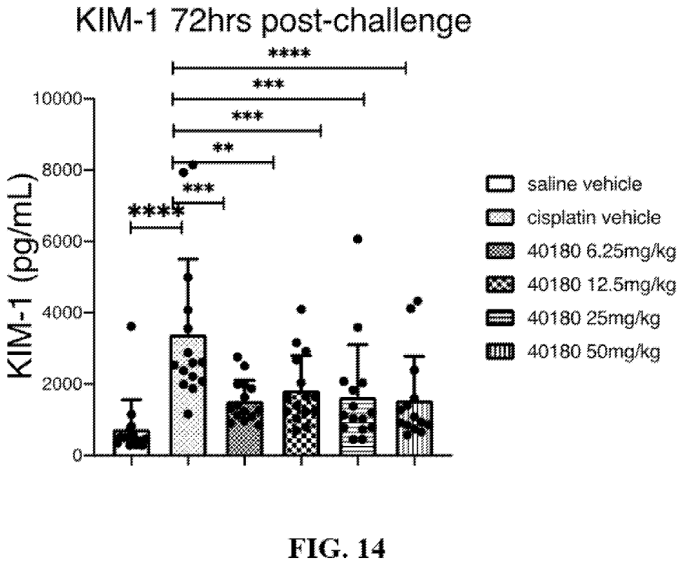

Referring to FIG. 14, mice (C57Bl/6) were challenged with a single intraperitoneal dose of 10 mg/kg cisplatin or saline and dosed by oral gavage once per day (QD) with 40180. On day 3 post-challenge, 40180 demonstrated a dose-dependent rescue of KIM-1 (kidney-injury marker 1), a urine biomarker specific for kidney injury.

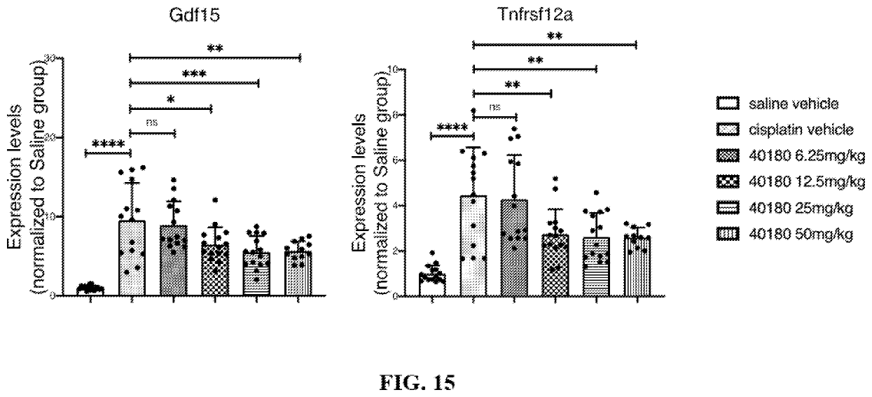

Referring to FIG. 15, mice (C57Bl/6) were challenged with a single intraperitoneal dose of 10 mg/kg cisplatin or saline and dosed by oral gavage, once per day (QD) with 40180 for three days. Cisplatin challenge significant increased expression of mitochondrial-stress related genes Gdf15 (left graph) and Tnfrsf12a (right graph); 40180 treatment reduced expression of these genes in a dose-responsive manner.

### 8. 35985 Restores Kidney Function Following Cisplatin Dosing

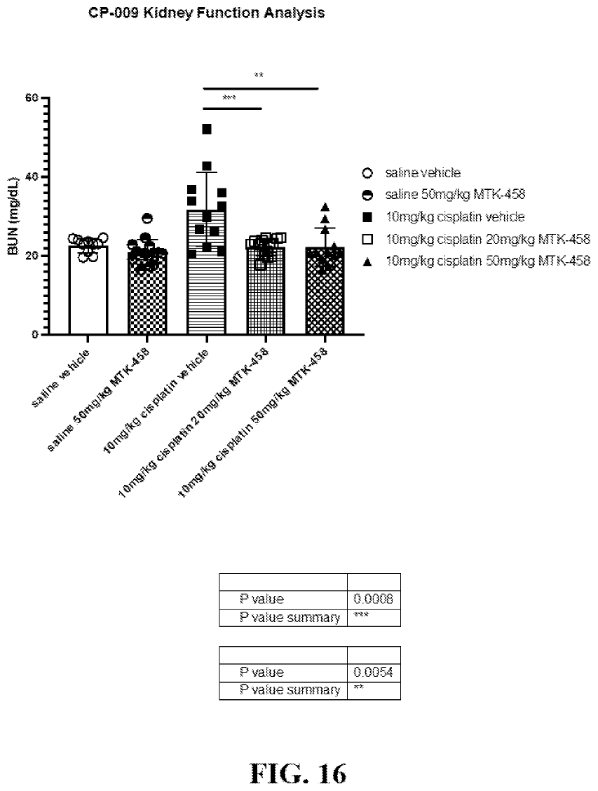

Referring to FIG. 16, administration of a single dose of cisplatin (10 mg/kg, i.p.) induces kidney dysfunction (illustrated by the increase in blood urea nitrogen, BUN, levels). Dysfunction was ameliorated by subsequent treatment with 20 mg/kg or 50 mg/kg MTK-458 (35985).

### 9. 40270 Reduces Kidney Injury Molecule 1 Following Cisplatin Dosing

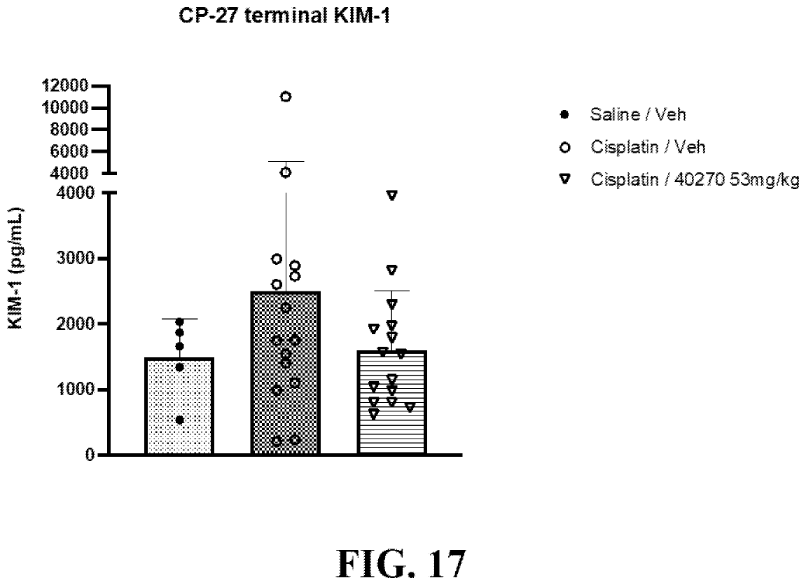

Referring to FIG. 17, administration of a single dose of cisplatin (10 mg/kg, i.p.) induces kidney dysfunction (illustrated by the increase in kidney injury molecule 1, KIM-1, levels). Dysfunction was ameliorated by subsequent treatment with 40270 (53 mg/kg p.o., b.i.d.).

### 10. 41088 Reduces Weight Loss Induced by Cisplatin

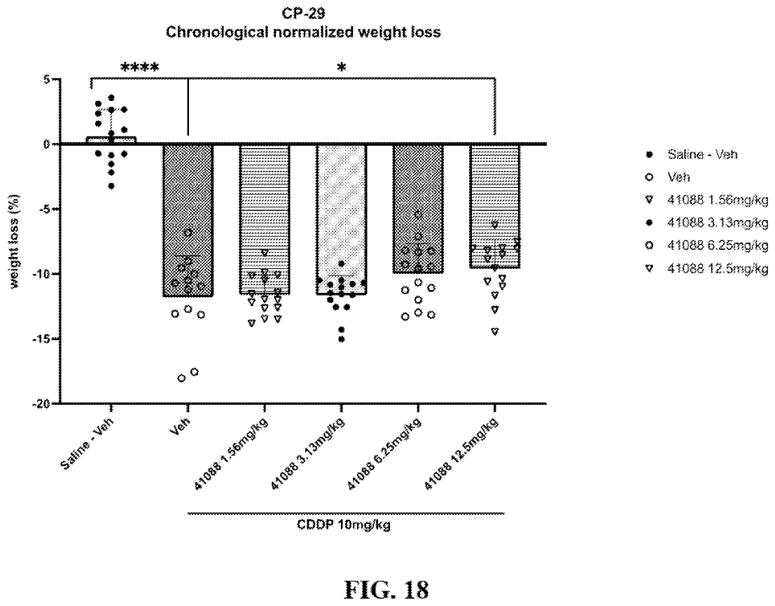

Referring to FIG. 18, administration of a single dose of cisplatin (10 mg/kg, i.p.) induces weight loss. Weight loss was ameliorated by subsequent treatment with 41088 (p.o., q.d.) at 1.56 mg/kg, 3.13 mg/kg, 6.25 mg/kg, and 12.5 mg/kg.

### 11. 41088 Restores Kidney Function Following Cisplatin Dosing

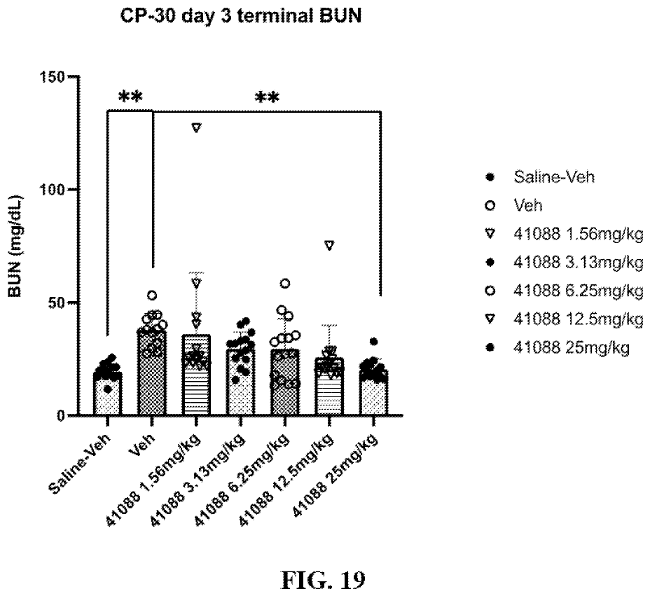

Referring to FIG. 19, administration of a single dose of cisplatin (10 mg/kg, i.p.) induces kidney dysfunction (illustrated by the increase in serum BUN levels). Dysfunction was ameliorated in a dose-dependent manner by subsequent treatment with 41088 (p.o., q.d.) at 1.56 mg/kg, 3.13 mg/kg, 6.25 mg/kg, 12.5 mg/kg, and 25 mg/kg.

### G. Additional Examples

### 1. Effect of MTK Compounds 35985 and 40180 on Cisplatin-Mediated Kidney Fibrosis Model

Repeated low-level tissue damage can lead to fibrosis and chronic disease in the affected tissue. Cisplatin can cause lung and kidney fibrosis in humans (Guinee et al., Cancer 1993), and repeated low-dose cisplatin challenge in mice causes fibrosis in mice (Sharp et al, AJPNephrology, 2016; Katagiri et al, Kidney International, 2015). By reducing cisplatin-mediated mtDNA damage, though a PINK1-dependent mechanism identical for evidence provided above, MTK compounds 35985 and 40180 will be shown to be protective for kidney fibrosis.

Sharp et al. describe a protocol by which mice (FVB strain) are injected weekly with 7 mg/kg cisplatin by intraperitoneal injection. N=12-15 mice per group will be injected with saline or 7 mg/kg cisplatin weekly for four weeks, and dosed with either vehicle or MTK compounds by oral gavage at doses of about 1 mg/kg, about 2 mg/kg, about 5 mg/kg, about 10 mg/kg, about 20 mg/kg, or about 50 mg/kg, either once a day or twice a day. Then, blood urea nitrogen or creatinine (urine), and kidney-injury marker-1 (KIM-1) will be assessed to evaluate kidney function and injury, respectively. Furthermore, quantitative PCR (qPCR) will be used to measure the expression of inflammatory markers such TNFalpha, IL-1beta, and IL-6. TGFbeta and fibronectin will be measured using western blot or commercially available ELISA kit as the principle readout for fibrosis, and count the number of infiltrating reactive immune cells in kidney sections by immunofluorescence or immunohistochemistry as a secondary measure of kidney fibrosis. Without wishing to be bound by theory, it is expected that administration of 35985 or 40180 will reduce fibrosis by 50% or more at therapeutic doses.

### 2. Effect of MTK Compounds 35985 and 40180 on Ischemia-Reperfusion Injury (Kidney)

Reperfusion injuries occur during tissue reoxygenation, and are most common following myocardial infarction, stroke, or surgery. While the causes of reperfusion injury are still unclear, recent evidence suggests that reactive oxygen species (ROS) from dysfunctional mitochondria play a pivotal role. Mitophagy is thought to play a key role in removing mitochondria depolarized during ischemia and PINK1-dependent mitophagy has been shown to oppose reperfusion injury (Livingston et al., Autophagy, 2019).

Livingston et al. describe a protocol by which mice (C57Bl/6) are surgerized to induce ischemia and reperfusion injury in kidney. Bilateral ischemia will be induced by clamping at the renal pedicles for 27 min, followed by reperfusion for 48 hr. Mice will be dosed with 35985 or 40180 by oral gavage at doses of about 1 mg/kg, about 2 mg/kg, about 5 mg/kg, about 10 mg/kg, about 20 mg/kg, or about 50 mg/kg beginning 1 hr prior to surgery, with additional doses once or twice daily until animals are sacrificed. There will be N=12-15 mice per group. Mitophagy induction will be tested using ELISA or Western blotting for pS65-Ub, and reperfusion injury will be assessed by assessing apoptosis by caspase 3 cleavage by Western blot, or TUNEL staining. Kidney function using blood urea nitrogen or creatinine (urine), and kidney-injury marker-1 (KIM-1) will be used to assess kidney function and injury, respectively. No injury conditions will have BUN at approximately 20-40 mg/dl, creatinine at 2 mg/dl, and KIM-1 at less than 500 μg/ml. Low injury will have BUN at approximately 50 mg/dl, creatinine at 3 mg/dl and KIM-1 approximately 1000 μg/ml. At high injury, BUN will be approximately 100-150 mg/dl, creatinine greater than 4 mg/dl, and KIM-1 at greater than 2000 μg/ml. Without wishing to be bound by theory, it is expected that administration of 35985 or 40180 will reduce apoptosis by approximately 30-50% or more at therapeutic doses.

### 3. Effect of MTK Compounds 35985 and 40180 on Ischemia-Reperfusion Injury (Heart)

Genetic deletion of PINK1 or PINK1-pathway member Parkin increases damage in myocardial infarction models, as measured by heart muscle mass and histology, eg. by Masson's trichdrome stain (Siddall et al, Plos ONE 2013; Kubli et al., J Biol Chem 2013). Accordingly, it is expected that increasing PINK1 function through administration of 35985 or 40180 will be protective in animal models of myocardial infarction.

Kubli et al. describe a protocol by which mice (C57Bl/6) are surgerized to induce myocardial infarction. Mice (N=12-15 per group) will be administered 35985 or 40180 at by oral gavage at doses of about 1 mg/kg, about 2 mg/kg, about 5 mg/kg, about 10 mg/kg, about 20 mg/kg, or about 50 mg/kg beginning 1 hr prior to surgery and daily or twice daily thereafter. Myocardial infarction is induced by permanently ligating the left anterior coronary artery by tightening an 8-0 silk suture around the coronary artery. Primary readouts will be survival at 7 days and extent of tissue damage, as assessed by histology compared to mock-surgerized mice. A secondary readout will be Seahorse respiration analysis (e.g. oxygen consumption rate) of cardiac mitochondria obtained from border zone or remote zone muscle 7 days after surgery. Border zone and remote zone muscle will be analyzed for mitophagy induction using pS65-Ub ELISA.

### 4. Effect of MTK Compounds 35985 and 40180 on Contrast-Induced Kidney Damage

Mitochondrial integrity and PINK1-dependent mitophagy are critical to kidney protection during contrast-induced imaging (Lin et al., Redox Biol., 2019). PINK1 knockout mice have worsened response in a model of contrast-induced acute kidney injury (CI-AKI).

Lin et al. describe a protocol by which mice (C57Bl/6) have induced CI-AKI. Mice have unilateral nephrectomy (removal of a single kidney) 3-weeks prior to the experiment. N=12-15 per group, mice are deprived of water for 24 hrs, and treated with furosemide (0.1 mg/kg, intraperitoneal) and either vehicle or 35985 or 40180 at by oral gavage at doses of about 1 mg/kg, about 2 mg/kg, about 5 mg/kg, about 10 mg/kg, about 20 mg/kg, or about 50 mg/kg 20 minutes prior to administration of the contrast agent, iohexol at 10 ul/g, by intravenous injection e.g. tail vein. Mice are sacrificed 24 hours after contrast agent injection. Mitophagy induction will be tested using ELISA or Western blotting for pS65-Ub, and reperfusion injury will be assessed by assessing apoptosis by caspase 3 cleavage by Western blot, or TUNEL staining. A 50% increase in cleaved caspase 3 may indicate apoptosis caused by reperfusion injury. Kidney function using blood urea nitrogen or creatinine (urine), and kidney-injury marker-1 (KIM-1) will be used to assess kidney function and injury, respectively. Without wishing to be bound by theory, it is expected that administration of 35985 or 40180 will reduce apoptosis by approximately 30-50% or more at therapeutic doses.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of treating a disorder associated with PINK1 in a subject in need thereof comprising administering to the subject an effective amount of a compound having a structure represented by a formula: 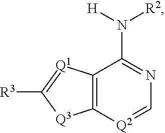
wherein Q¹ is N or CH and R³ is a 3- to 6-membered cycloalkyl, C1-C6 haloalkyl, C1-C6 haloalkoxy, or C1-C6 halohydroxyalkyl;
or wherein Q¹ is CR¹ and R³ is hydrogen;
wherein R¹ is selected from C1-C6 haloalkyl, C1-C6 haloalkoxy, C1-C6 halohydroxyalkyl, and a structure represented by a formula: 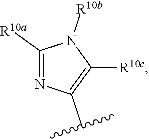
wherein each of R^{10a}, R^{10b}, and R^{10c}, when present, is independently selected from hydrogen and C1-C4 alkyl;
wherein Q² is N or CH;
wherein Q³ is CH₂ or NH;
wherein R² is selected from C1-C6 alkyl, —CR^{11a}R^{11b}Cy¹, or Cy¹;
wherein each of R^{11a} and R^{11b}, when present, is independently selected from hydrogen, C1-C5 alkyl, and C1-C4 hydroxyalkyl;
or wherein each of R^{11a} and R^{11b} together comprise a 3-membered cycloalkyl;
wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)˜Cy², and Cy²;
wherein n, when present, is 0, 1, or 2;
wherein R¹², when present, is selected from hydrogen and C1-C4 alkyl;
wherein Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
provided that when Q¹ is CR¹, R¹ is C1-C6 haloalkyl, and R² is Cy¹, then Cy¹ is not a 6-membered carbocycle or a 9-membered heteroaryl, and
provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl,
or a pharmaceutically acceptable salt thereof.

2. The method of claim 1, wherein Cy¹, when present, is a structure represented by a formula selected from: 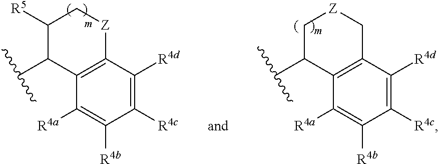
wherein m is 0 or 1;
wherein Z is O, CR^{13a}R^{13b}, or NR¹⁴;
wherein each of R^{13a} and R^{13b}, when present, is independently selected from hydrogen, halogen, —OH, and C1-C4 alkoxy,
or wherein each of R^{13a} and R^{13b}, when present, together comprise ═O; and
wherein R¹⁴, when present, is selected from —C(O)(C1-C4 alkyl), C1-C4 alkyl, and C2-C4 alkenyl;
wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
wherein R⁵, when present, is selected from hydrogen, —O(CH₂)ₙCy², —NR¹²(CH₂)ₙCy², and Cy².

3. The method of claim 1, wherein the compound has a structure represented by a formula: 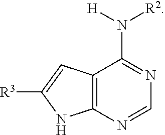
The method of claim 1, wherein the compound has a structure represented by a formula: 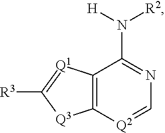
wherein Q is N or CH and R³ is a 3- to 6-membered cycloalkyl, C1-C6 haloalkyl, C1-C6 haloalkoxy, or C1-C6 halohydroxyalkyl;
or wherein Q¹ is CR¹ and R³ is hydrogen;
or wherein R¹ is selected from C1-C6 haloalkyl, C1-C6 haloalkoxy, C1-C6 halohydroxyalkyl, and a structure represented by a formula: 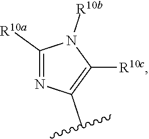
wherein each of R^{10a}, R^{10b}, and R^{10c}, when present, is independently selected from hydrogen and C1-C4 alkyl;
wherein Q² is N or CH;
wherein Q³ is CH₂ or NH;
wherein R² is Cy,
wherein Cy¹ is a structure represented by a formula selected from: 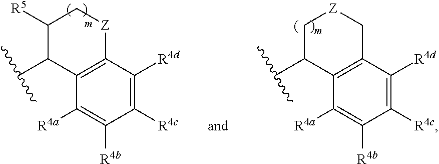
wherein m is 0 or 1;
wherein Z is O, CR^{13a}R^{13b}, or NR¹⁴;
wherein each of R^{13a} and R^{13b}, when present, is independently selected from hydrogen, halogen, —OH, and C1-C4 alkoxy,
or wherein each of R^{13a} and R^{13b}, when present, together comprise ═O; and
wherein R¹⁴, when present, is selected from —C(O)(C1-C4 alkyl), C1-C4 alkyl, and C2-C4 alkenyl;
wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
wherein R⁵, when present, is selected from hydrogen, —O(CH₂)ₙCy², —NR¹²(CH₂)Cy², and Cy²;
wherein Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
provided that when Q¹ is CR¹, R¹ is C1-C6 haloalkyl, and R² is Cy¹, then Cy¹ is not a 6-membered carbocycle or a 9-membered heteroaryl, and
or a pharmaceutically acceptable salt thereof.

5. The method of claim 1, wherein the compound has a structure represented by a formula:

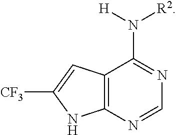

6. (canceled)

7. The method of claim 1, wherein the compound has a structure represented by a formula: 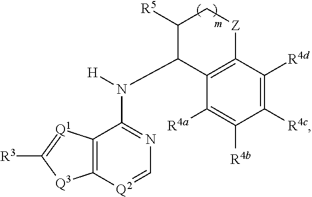
wherein m is 0 or 1;
wherein Q¹ is N or CH and R³ is a 3- to 6-membered cycloalkyl, C1-C6 haloalkyl, C1-C6 haloalkoxy, or C1-C6 halohydroxyalkyl;
wherein Z is CR^{13a}R^{13b} or O;
wherein each of R^{13a} and R^{13b}, when present, is independently selected from hydrogen, halogen, —OH, and C1-C4 alkoxy,
or wherein each of R^{13a} and R^{13b}, when present, together comprise ═O;
wherein each of R^{4a}, R^{4b}, R^{4c}, and R^{4d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
wherein R⁵ is selected from —O(CH₂)˜Cy², —NR¹²(CH₂)ₙCy², and Cy².

8.-9. (canceled)

10. The method of claim 1, wherein the compound is selected from:

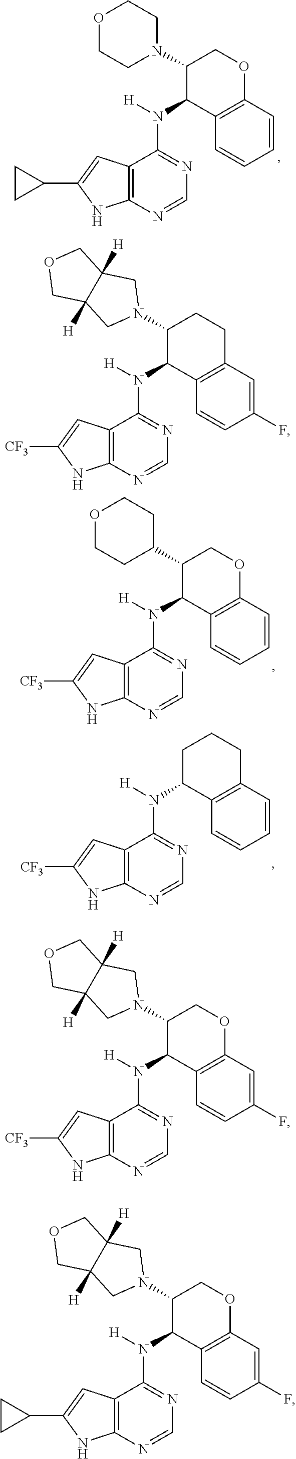

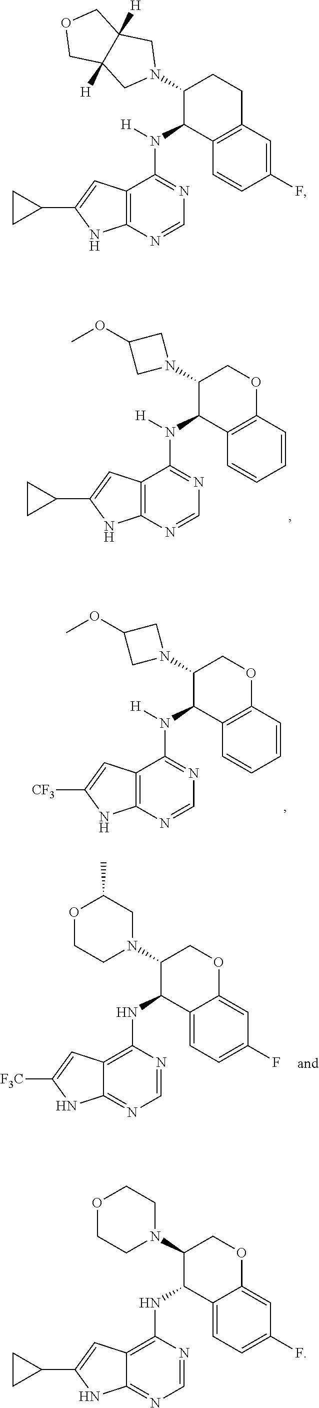

11. The method of claim 1, wherein the subject is a mammal.

12. The method of claim 1, wherein the subject is a human.

13. (canceled)

14. The method of claim 1, wherein the administering is via oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

15.-16. (canceled)

17. The method of claim 1, further comprising administering an effective amount of an agent associated with the treatment of a kidney disease, a fibrotic disorder, or a reperfusion injury.

18.-22. (canceled)

23. The method of claim 14, wherein the compound and the agent are administered simultaneously or sequentially.

24.-25. (canceled)

26. The method of claim 1, wherein the disorder associated with PINK1 is a kidney disease, a fibrotic disorder, or a reperfusion injury.

27. The method of claim 1, wherein the disorder associated with PINK1 is chronic kidney disease selected from autosomal dominant polycystic kidney disease, diabetic nephropathy, hypertension-induced renal injury, crescentic glomerulonephritis, membranous nephropathy, membranous nephropathy, IgA nephropathy, amyloid A amyloidosis, and secondary nephrotic syndrome.

28. (canceled)

29. The method of claim 1, wherein the disorder associated with PINK1 is pulmonary fibrosis, liver fibrosis, heart fibrosis, mediastinal fibrosis, retroperitoneal cavity fibrosis, bone marrow fibrosis, skin fibrosis, scleroderma, pancreatic fibrillation, prostatic hyperplasia caused by fibrillation, and renal fibrosis.

30-35. (canceled)

36. A kit comprising a compound having a structure represented by a formula: 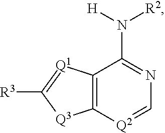
wherein Q1 is N or CH and R3 is a 3- to 6-membered cycloalkyl, C1-C6 haloalkyl, C1-C6 haloalkoxy, or C1-C6 halohydroxyalkyl;
or wherein Q1 is CR1 and R3 is hydrogen;
wherein R1 is selected from C1-C6 haloalkyl, C1-C6 haloalkoxy, C1-C6 halohydroxyalkyl, and a structure represented by a formula: 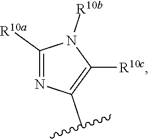
wherein each of R10a, R10b, and R10c, when present, is independently selected from hydrogen and C1-C4 alkyl;
wherein Q2 is N or CH;
wherein Q3 is CH2 or NH;
wherein R2 is selected from C1-C6 alkyl, —CR11aR11bCy1, or Cy1;
wherein each of R11a and R11b, when present, is independently selected from hydrogen, C1-C5 alkyl, and C1-C4 hydroxyalkyl;
or wherein each of R11a and R11b together comprise a 3-membered cycloalkyl;
wherein Cy1, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH2, —OH, —NO2, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH2)nCy2, —NR12(CH2)nCy2, and Cy2;
wherein n, when present, is 0, 1, or 2;
wherein R12, when present, is selected from hydrogen and C1-C4 alkyl;
wherein Cy2 is a C3-C9 heterocycle having at least one O, S, or N atom and substituted with 0, 1, 2, or 3 groups independently selected from halogen, —CN, —NH2, —OH, —NO2, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
provided that when Q1 is CR1, R1 is C1-C6 haloalkyl, and R2 is Cy1, then Cy1 is not a 6-membered carbocycle or a 9-membered heteroaryl, and
provided that when R2 is —CR11aR11bCy1 or Cy1, one or both of R11a and R11b, when present, is hydrogen, and Cy1 is a 6-membered aryl or furanyl, then Q1 is CH and R3 is not a C1-C6 haloalkyl,
or a pharmaceutically acceptable salt thereof, and one or more of:
a. an agent associated with the treatment of a kidney disease or a fibrotic disorder;
b. an agent associated with the treatment of a reperfusion injury;
c. instructions for administering the compound in connection with treating a kidney disease, a fibrotic disorder, and/or a reperfusion injury; and
d. instructions for treating a kidney disease, a fibrotic disorder, and/or a reperfusion injury.

37. (canceled)

38. The kit of claim 36, wherein the compound and the agent associated with the treatment of a kidney disease or a fibrotic disorder are co-packaged.

39. The kit of claim 36, wherein the compound and the agent associated with the treatment of a kidney disease or a fibrotic disorder are co-formulated.

40-42. (canceled)

43. A method of treating ototoxicity in a subject in need thereof comprising administering to the subject an effective amount of a compound having a structure represented by a formula: 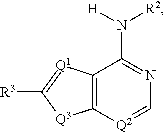
wherein Q¹ is N or CH and R³ is a 3- to 6-membered cycloalkyl, C1-C6 haloalkyl, C1-C6 haloalkoxy, or C1-C6 halohydroxyalkyl;
or wherein Q¹ is CR¹ and R³ is hydrogen;
wherein R¹ is selected from C1-C6 haloalkyl, C1-C6 haloalkoxy, C1-C6 halohydroxyalkyl, and a structure represented by a formula: 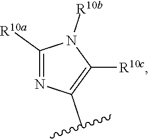
wherein each of R^{10a}, R^{10b}, and R^{10c}, when present, is independently selected from hydrogen and C1-C4 alkyl;
wherein Q² is N or CH;
wherein Q³ is CH₂ or NH;
wherein R² is selected from C1-C6 alkyl, —CR^{11a}R^{11b}Cy¹, or Cy¹;
wherein each of R^{11a} and R^{11b}, when present, is independently selected from hydrogen, C1-C5 alkyl, and C1-C4 hydroxyalkyl;
or wherein each of R^{11a} and R^{11b} together comprise a 3-membered cycloalkyl;
wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, —O(CH₂)ₙCy², —NR¹²(CH₂)ₙCy², and Cy²;
wherein n, when present, is 0, 1, or 2;
wherein R¹², when present, is selected from hydrogen and C1-C4 alkyl;
wherein Cy² is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
provided that when Q¹ is CR¹, R¹ is C1-C6 haloalkyl, and R² is Cy¹, then Cy¹ is not a 6-membered carbocycle or a 9-membered heteroaryl, and
provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl,
or a pharmaceutically acceptable salt thereof.

44. The method of claim 43, wherein the otoxicity is cisplatin-induced otoxicity.

45. The method of claim 1, wherein the compound is selected from:

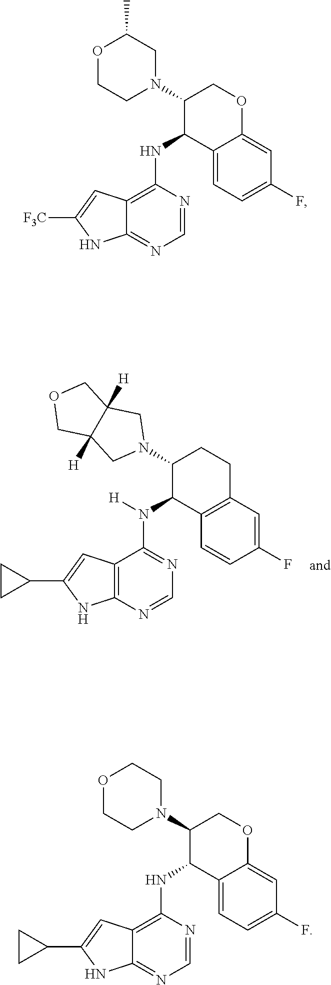 [SMILES: C[C@@H]1CN([C@@H]2COc3cc(F)ccc3[C@H]2Nc2ncnc3[nH]c(C(F)(F)F)cc23)CCO1 ; Fc1ccc2c(c1)CC[C@@H](N1C[C@H]3COC[C@H]3C1)[C@@H]2Nc1ncnc2[nH]c(C3CC3)cc12 ; Fc1ccc2c(c1)OC[C@H](N1CCOCC1)[C@H]2Nc1ncnc2[nH]c(C3CC3)cc12]

46. The method of claim 43, wherein the compound is selected from: 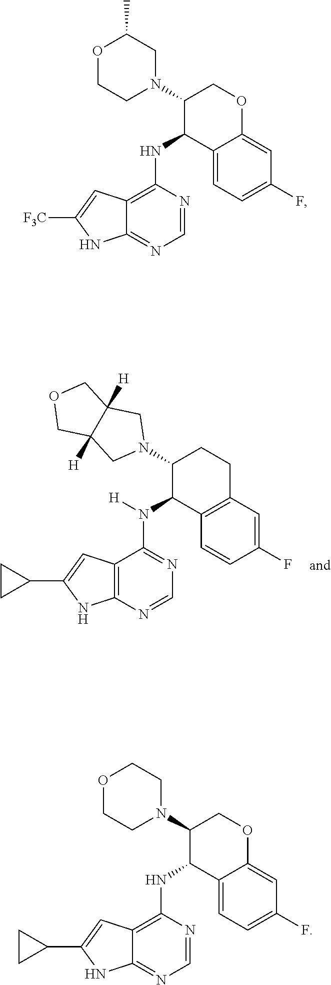 [SMILES: C[C@@H]1CN([C@@H]2COc3cc(F)ccc3[C@H]2Nc2ncnc3[nH]c(C(F)(F)F)cc23)CCO1 ; Fc1ccc2c(c1)CC[C@@H](N1C[C@H]3COC[C@H]3C1)[C@@H]2Nc1ncnc2[nH]c(C3CC3)cc12 ; Fc1ccc2c(c1)OC[C@H](N1CCOCC1)[C@H]2Nc1ncnc2[nH]c(C3CC3)cc12]


SEQUENCE LISTING (16 sequences)

SEQ ID NO: 1 - DNA, 20 bp, Artificial Sequence
  Oligo 1
gtgttgggattcggcttggt

SEQ ID NO: 2 - DNA, 21 bp, Artificial Sequence
  Oligo 2
gtccatgcacttgtcgaggtc

SEQ ID NO: 3 - DNA, 23 bp, Artificial Sequence
  Oligo 3
gaggattttgctaacctgacacc

SEQ ID NO: 4 - DNA, 23 bp, Artificial Sequence
  Oligo 4
gaggattttgctaacctgacacc

SEQ ID NO: 5 - DNA, 21 bp, Artificial Sequence
  Oligo 5
gcacatccatcggtcatccag

SEQ ID NO: 6 - DNA, 22 bp, Artificial Sequence
  Oligo 6
gccacagtcaaaccttcttcaa

SEQ ID NO: 7 - DNA, 20 bp, Artificial Sequence
  Oligo 7
ctggcaatgcctgaacaacg

SEQ ID NO: 8 - DNA, 20 bp, Artificial Sequence
  Oligo 8
ggtcgggacttggttctgag

SEQ ID NO: 9 - DNA, 19 bp, Artificial Sequence
  Oligo 9
gggcatcctgaccctcaag

SEQ ID NO: 10 - DNA, 20 bp, Artificial Sequence
  Oligo 10
tccatgtcgtcccagttggt

SEQ ID NO: 11 - DNA, 22 bp, Artificial Sequence
  Oligo 11
ccgcaagggaaagatgaaagac

SEQ ID NO: 12 - DNA, 20 bp, Artificial Sequence
  Oligo 12
tcgtttggtttcggggtttc

SEQ ID NO: 13 - DNA, 20 bp, Artificial Sequence
  Oligo 13
ctagcagaaacaaaccgggc

SEQ ID NO: 14 - DNA, 20 bp, Artificial Sequence
  Oligo 14
ccggctgcgtattctacgtt

SEQ ID NO: 15 - DNA, 24 bp, Artificial Sequence
  Oligo 15
gccagcctctcctgattttagtgt

SEQ ID NO: 16 - DNA, 24 bp, Artificial Sequence
  Oligo 16
gggaacacaaaagacctcttctgg